SEQUENCE LISTING (41 sequences)

SEQ ID NO: 1 - DNA, 55 bp, Artificial Sequence
  Hairpin-loop DNA aptamer sensor (TMP-1)
gggtagggcgggttgggaaatattcctgggggagtattgcggaggaaggtttccc

SEQ ID NO: 2 - DNA, 55 bp, Artificial Sequence
  Hairpin-loop DNA aptamer sensor (TMP-5)
gggtagggcgggttgggagctattcctgggggagtattgcggaggaagggatccc

SEQ ID NO: 3 - DNA, 55 bp, Artificial Sequence
  Hairpin-loop DNA aptamer sensor (TMP-6)
gggtagggcgggttgggatatgttcctgggggagtattgcggaggaaggtatccc

SEQ ID NO: 4 - DNA, 56 bp, Artificial Sequence
  Hairpin-loop DNA aptamer sensor (9A)
gggtagggcgggttgggcattggttcctgggggagtattgcggaggaaggatgccc

SEQ ID NO: 5 - DNA, 28 bp, Artificial Sequence
  Hairpin-loop DNA aptamer sensor (9B)
gaccagggcaaacggtaggtgagtggtc

SEQ ID NO: 6 - DNA, 56 bp, Artificial Sequence
  Hairpin-loop DNA aptamer sensor (10A)
gggtagggcgggttgggtctcttatcctgggggagtattgcggaggaaggagaccc

SEQ ID NO: 7 - DNA, 55 bp, Artificial Sequence
  Hairpin-loop DNA aptamer sensor (10B)
gggtagggcgggttggggacctatcctgggggagtattgcggaggaagggttccc

SEQ ID NO: 8 - DNA, 55 bp, Artificial Sequence
  Hairpin-loop DNA aptamer sensor (10C)
gggtagggcgggttgggagtccgtcctgggggagtattgcggaggaaggaatccc

SEQ ID NO: 9 - DNA, 56 bp, Artificial Sequence
  Hairpin-loop DNA aptamer sensor (10D)
gggtagggcgggttgggagccgtttcctgggggagtattgcggaggaagggatccc

SEQ ID NO: 10 - DNA, 56 bp, Artificial Sequence
  Hairpin-loop DNA aptamer sensor (10E)
gggtagggcgggttgggtatctcttcctgggggagtattgcggaggaaggacaccc

SEQ ID NO: 11 - DNA, 56 bp, Artificial Sequence
  Hairpin-loop DNA aptamer sensor (10F)
gggtagggcgggttgggcatcctatcctgggggagtattgcggaggaaggaggccc

SEQ ID NO: 12 - DNA, 55 bp, Artificial Sequence
  Hairpin-loop DNA aptamer sensor (TMP5-1)
gggtagggcgggttgggagcttatcctgggggagtattgcggaggagaggatccc

SEQ ID NO: 13 - DNA, 55 bp, Artificial Sequence
  Hairpin-loop DNA aptamer sensor (TMP5-2)
gggtagggcgggttgggagctcatcctgggggagtattgcggaggaggggatccc

SEQ ID NO: 14 - DNA, 55 bp, Artificial Sequence
  Hairpin-loop DNA aptamer sensor (TMP5-5)
gggtagggcgggttgggagctatccctgggggagtattgcggagggagggatccc

SEQ ID NO: 15 - DNA, 55 bp, Artificial Sequence
  Hairpin-loop DNA aptamer sensor (TMP5-TG1)
gggtagggcgggttgggagctattcctgggggagtattgcggaggaggagatccc

SEQ ID NO: 16 - DNA, 17 bp, Artificial Sequence
  DNAzyme with a peroxidase activity
gggtagggcgggttggg

SEQ ID NO: 17 - DNA, 25 bp, Artificial Sequence
  AMP aptamer
cctgggggagtattgcggaggaagg

SEQ ID NO: 18 - DNA, 28 bp, Artificial Sequence
  Arginine aptamer
gaccagggcaaacggtaggtgagtggtc

SEQ ID NO: 19 - DNA, 58 bp, Artificial Sequence
  Hairpin-loop DNA aptamer sensor (TMP-5Arg)
gggtagggcgggttgggagcgaacgaccagggcaaacggtaggtgagtggtcgatccc

SEQ ID NO: 20 - DNA, 53 bp, Artificial Sequence
  Hairpin-loop DNA aptamer sensor (AMP Contr.)
gggtagggcgggttgggaaccttcctgggggagtattgcggaggaaggttccc

SEQ ID NO: 21 - DNA, 60 bp, Artificial Sequence
  First-Candidate 5
gggtagggcgggttgggagctattcctgcgaatcagtgcgacatccgccgaagggatccc

SEQ ID NO: 22 - DNA, 60 bp, Artificial Sequence
  First-Candidate 6
gggtagggcgggttgggagctattccttagcgtaccacttcaggcatcggaagggatccc

SEQ ID NO: 23 - DNA, 60 bp, Artificial Sequence
  Second-Candidate 7
gggtagggcgggttgggagctattcctgcgggcgctgttcgcctagtcggaagggatccc

SEQ ID NO: 24 - DNA, 30 bp, Artificial Sequence
  Patulin aptemer region of First-Candidate 5
cctgcgaatcagtgcgacatccgccgaagg

SEQ ID NO: 25 - DNA, 30 bp, Artificial Sequence
  Patulin aptamer region of First-Candidate 6
ccttagcgtaccacttcaggcatcggaagg

SEQ ID NO: 26 - DNA, 30 bp, Artificial Sequence
  Patulin aptamer region of Second-Candidate 7
cctgcgggcgctgttcgcctagtcggaagg

SEQ ID NO: 27 - DNA, 77 bp, Artificial Sequence
  Template DNA for patulin RNA aptamer
gggcaacctacggctttcaccgtttcgnnnnnnnnnnnnnnnnnnnnnnnnnnnnnnnnnnnctcatcagggtcgcc

SEQ ID NO: 28 - DNA, 33 bp, Artificial Sequence
  Forward primer
taatacgactcactatagggcgaccctgatgag

SEQ ID NO: 29 - DNA, 27 bp, Artificial Sequence
  Reverse primer
gggcaacctacggctttcaccgtttcg

SEQ ID NO: 30 - RNA, 78 nt, Artificial Sequence
  Ribozyme comprising patulin RNA aptamer region
gggcgacccugaugagaaagaucuacagcaaaaaccauaguaguaaagaagcgaaacggugaaagccguagguugccc

SEQ ID NO: 31 - RNA, 78 nt, Artificial Sequence
  Ribozyme comprising patulin aptamer region
gggcgacccugaugagaguauaaaauaucaaugaaauaaacaagccauuaucgaaacggugaaagccguagguugccc

SEQ ID NO: 32 - RNA, 78 nt, Artificial Sequence
  Ribozyme comprising patulin RNA aptamer region
gggcgacccugaugagggggcacgcguacggcuagccaagucaaacgauuccgaaacggugaaagccguagguugccc

SEQ ID NO: 33 - RNA, 35 nt, Artificial Sequence
  Patulin RNA aptamer
aaagaucuacagcaaaaaccauaguaguaaagaag

SEQ ID NO: 34 - RNA, 35 nt, Artificial Sequence
  patulin RNA aptamer 33
aguauaaaauaucaaugaaauaaacaagccauuau

SEQ ID NO: 35 - RNA, 35 nt, Artificial Sequence
  Patulin RNA aptamer
ggggcacgcguacggcuagccaagucaaacgauuc

SEQ ID NO: 36 - RNA, 31 nt, Artificial Sequence
  5′ deleted patulin RNA aptamer
uaaaauaucaaugaaauaaacaagccauuau

SEQ ID NO: 37 - RNA, 31 nt, Artificial Sequence
  5′ deleted patulin RNA aptamer
cacgcguacggcuagccaagucaaacgauuc

SEQ ID NO: 38 - RNA, 74 nt, Artificial Sequence
  5′ deleted Ribozyme
gggcgacccugaugaguaaaauaucaaugaaauaaacaagccauuaucgaaacggugaaagccguagguugccc

SEQ ID NO: 39 - RNA, 74 nt, Artificial Sequence
  5′ deleted ribozyme
gggcgacccugaugagcacgcguacggcuagccaagucaaacgauuccgaaacggugaaagccguagguugccc

SEQ ID NO: 40 - DNA, 61 bp, Artificial Sequence
  SC-7-CCCA
gggtagggcgggttgggagctattcctgcgggcgctgttcgcctagtcggaagggatccca

SEQ ID NO: 41 - DNA, 61 bp, Artificial Sequence
  SC-7-CCCA-TMP-7
gggtagggcgggttgggctgtcgtcctgcgggcgctgttcgcctagtcggaaggtagccca

(19) United States
(12) Patent Application Publication
(10) Pub. No.: US 2015/0292005 A1
(43) Pub. Date: Oct. 15, 2015

(54) NUCLEIC ACID MOLECULES FOR HIGHLY SENSITIVE DETECTION OF LIGANDS, SCREENING METHOD FOR NUCLEIC ACID MOLECULES, AND OPTIMIZATION METHOD FOR SENSITIVITY OF NUCLEIC ACID MOLECULES

(71) Applicant: KIRIN KABUSHIKI KAISHA, Nakano-ku, Tokyo (JP)

(72) Inventors: Yasuyuki TOMITA, Tokyo (JP); Yuji MORITA, Tokyo (JP); Daisuke FUJIWARA, Tokyo (JP)

(73) Assignee: KIRIN KABUSHIKI KAISHA, Nakano-ku, Tokyo (JP)

(21) Appl. No.: 14/397,033

(22) PCT Filed: Apr. 25, 2013
(86) PCT No.: PCT/JP2013/062289

§ 371 Date: Oct. 24, 2014

(30) Foreign Application Priority Data
Apr. 27, 2012 (JP) 2012-103864
Sep. 6, 2012 (JP) 2012-196462

(51) Int. Cl. C12Q 1/68

(52) U.S. Cl. CPC C12Q 1/6825; C12Q 1/6834

(57) ABSTRACT

It is an object of the present invention to provide nucleic acid molecules that enable highly sensitive detection of ligands (e.g., patulin). It is another object of the present invention to provide a screening method for nucleic acid molecules that enable highly sensitive detection of ligands (e.g., patulin), and a method for screening for nucleic acid molecules used for the optimization of nucleic acid molecules that enable highly sensitive detection of ligands (e.g., patulin). It is a further object of the present invention to provide a method for effectively removing ligands from samples containing ligands (e.g., patulin). According to the present invention, there is provided a loop-structured nucleic acid molecule for detection of ligands (e.g., patulin) having a DNA aptamer and a DNAzyme, wherein the sequence is modified between the DNA aptamer region and the DNAzyme.

### TECHNICAL FIELD

The present invention relates to nucleic acid molecules for detection of ligands. The present invention also relates to a screening method for such nucleic acid molecules and an optimization method for sensitivity of nucleic acid molecules.

### BACKGROUND ART

Antibodies and aptamers are known as molecules having activity to specifically bind to target molecules. Antibodies are superior in that antigen-specific antibodies can be obtained by a simple method, and have been widely used in detection of antigens, etc. Meanwhile, aptamers are difficult to be designed, but are relatively easily synthesized and can be obtained by a completely artificial method. Aptamers are superior to antibodies in that molecules having specificity for a molecule for which it is difficult to prepare antibodies, for example, molecules binding to an antigen having toxicity or a molecule having low antigenicity (e.g., small molecule compounds), can be obtained, that aptamers can be inexpensively manufactured, and that aptamers can be stably stored in the dry state, etc.

Detection of the binding of an aptamer to its target molecule is generally achieved by detection of the structural change of the aptamer, especially if the labeling of the target molecule is difficult. For example, as a method for detecting the structural change of aptamers, a method for detecting the self-cleavage of aptamers caused by the structural change is known. For example, in the case of self-cleaving RNA aptamer, when an aptamer binds to its target molecule, the self-cleaving activity is activated and the molecule is cleaved, and by detecting the fragments of this cleaved RNA aptamer, the binding of the aptamer to the target molecule can be monitored. In this way, in aptamers, target molecules are detected by monitoring the generation of signals dependent on the binding to the target molecules (in the case of the above mentioned case, the production of RNA fragments by self-cleaving activity).

In comparison of DNA with RNA, RNA is known to be structurally more flexible, and all nucleic acids having an enzymatic activity discovered in vivo are RNAs. On the other hand, although structural flexibility is lacking, DNA is superior in chemical stability and plays a role in preservation of genetic information, etc. Thus, most of aptamers are RNA molecules having an ability to form a flexible conformation and exerting various functions. However, aptamers using DNA molecules have also recently been reported (Non-Patent Literature 1). Non-Patent Literature 1 discloses hairpin-loop-structured DNA aptamers, and when AMP as a ligand binds to the DNA aptamer, the secondary structure of the whole molecule is changed and the oxidoreductase activity of the molecule is expressed. Although measurement of the enzymatic activity enables detection of ligands, the detection sensitivity is not so high.

Highly sensitive aptamers are usually obtained by screening aptamers using sensitivity as an index from the aptamer candidate group obtained by randomly modifying the sequence by a molecular evolution method (Patent Literature 1, 2, and 3), and generally, it is not easy to design aptamers for high sensitivity, and guidelines for the design and conditions for high sensitivity of DNA aptamers are hardly known.

Patulin is a type of mycotoxin produced by Penicillium expansum and Aspergillus oryzae, and known to be detected from rotten apples. With regard to the toxicity of patulin, patulin has been shown to have genetic toxicity as well as organ hemorrhagic toxicity due to the inhibition of cell membrane permeability, and carcinogenic potential has been suggested by animal experiments. Thus, the amount of patulin in apple products is used as a product quality standard. Methods for detection and selective removal of patulin are highly important, and thus it is expected that if a substance specifically binding to patulin is obtained, the quality of apple products such as juice can be simply tested and patulin can be effectively removed from a product.

Aptamers can be fabricated by the SELEX (systematic evolution of ligands by exponential enrichment) method (Patent Literature 4). In the SELEX method, nucleic acid molecules specifically binding to target substances are obtained from a pool of RNA or DNA having sequence diversity of about 10¹⁴ sequences. Nucleic acid molecules in a nucleic acid pool used in the SELEX method generally have a structure in which random sequences of about 20 to 40 residues are sandwiched between primer sequences. In the SELEX method, this nucleic acid pool is brought into contacted with target substances to recover only nucleic acids bound to the target substances. If recovered nucleic acids are RNAs, they are amplified by RT-PCR, while if recovered nucleic acids are DNAs, they themselves are used as a template in PCR to be amplified. By transcribing RNAs from the amplified DNAs as needed or by using the DNAs themselves, nucleic acid molecules specifically binding to target substances are further obtained. In the SELEX method, usually this process is repeated about 10 times to obtain aptamers specifically binding to target substances.

In the general SELEX method, target substances of aptamers are immobilized on carriers, and nucleic acids having an affinity for the target substances are recovered by utilizing the affinity for the target substances. Meanwhile, Breakers et al. proposed a method for performing the SELEX method without immobilization of target substances on carriers (Non-Patent Literature 2). In the method by Breakers et al., specifically, by connecting self-cleaving ribozymes with random sequences and by screening RNAs that exert self-cleaving activity only in the presence of target substances, RNAs binding to target substances are obtained from the random sequences.

Furthermore, a method in which aptamers are screened with microarrays has been developed (Non-Patent Literature 3). Screening by microarrays is an extremely useful method in terms of enabling screening a large amount of molecules at once, but target molecules are mainly limited to molecules that can be fluorescently labeled such as proteins. It was difficult to apply this method to, for example, molecules that are difficult to be fluorescently labeled or small molecule compounds of which physical properties are greatly changed by labeling.

### REFERENCE LIST

### Patent Document

### [Patent Document 1]

Japanese Patent Laid-Open Publication No. 2003-512059

### [Patent Document 2]

WO 2012/86772

### [Patent Document 3]

WO 2013/5723

### [Patent Document 4]

WO 1991/19813

### Non Patent Document

### [Non Patent Document 1]

Teller C., Shimron S., Willner I., Aptamer-DNAzynne hairpins for amplified biosensing. Anal. Chem. (2009) 81:9114-9119

### [Non Patent Document 2]

Nature structural biology 6, 1062-1071, 1999

### [Non Patent Document 3]

Nucleic Acids Research, Vol. 37, No. 12 e87, 2009

### SUMMARY OF THE INVENTION

### Technical Problem

It is an object of the present invention to provide nucleic acid molecules that enable highly sensitive detection of ligands (e.g., patulin). It is another object of the present invention to provide a screening method for nucleic acid molecules that enable highly sensitive detection of ligands (e.g., patulin), and a screening method for nucleic acid molecules used for the optimization of nucleic acid molecules that enable highly sensitive detection of ligands (e.g., patulin). It is a further object of the present invention to provide a method for effectively removing ligands from samples containing ligands (e.g., patulin).

### Solution to the Problem

The present inventors have found that in DNA molecules forming a loop structure having a DNA aptamer region and a DNAzyme region, when a sequence intervenes between the DNA aptamer region and the DNAzyme region, the sequence has a specific rule in DNA molecules that are highly sensitive to ligands. The present inventors have also found that a large amount of hairpin-loop-structured DNA molecules for highly sensitive detection of ligands can be rapidly and simply screened by using microarrays by electrochemical detection methods. The present inventors have also found DNA molecules and RNA molecules for highly sensitive detection of patulin, a small molecule compound, as a ligand. The present invention is an invention made based on these findings.

In other words, according to the present invention, the following inventions are provided:

(1) A DNA construct forming a loop structure or a nucleic acid construct having a base sequence equivalent thereto, which includes a DNA aptamer region, an aptamer mask region, a junction region 1, a junction region 2, an effector region, and a terminal region,

each region being connected in the order of the junction region 1, the aptamer mask region, the DNA aptamer region, and the junction region 2 from the 5′ side of the DNA construct,

at least part of the effector region being inactivated by being hybridized with the terminal region in the absence of ligands to the DNA aptamer region, and

the effector region being activated dependent on the binding of ligands to the DNA aptamer region;

wherein

4 to 7 bases at the 3′ end of the DNA aptamer region are hybridized with the aptamer mask region of 3 to 5 bases length adjacent to the 5′ side of the DNA aptamer region in the absence of ligands, to form a total of 4 to 11 hydrogen bonds between bases in the hybridized region;

the junction region 2 of 1 to 5 bases length adjacent to the 3′ side of the DNA aptamer region is hybridized with the junction region 1 adjacent to the 5′ side of the aptamer mask region in the absence of ligands, to form a total of 3 or more hydrogen bonds between bases in the hybridized region; and

the effector region is adjacent to the 5′ side of the junction region 1 and the terminal region is adjacent to the 3′ side of the junction region 2, or the effector region is adjacent to the 3′ side of the junction region 2 and the terminal region is adjacent to the 5′ side of the junction region 1.

(2) A DNA construct forming a loop structure or a nucleic acid construct having a base sequence equivalent thereto, which includes a DNA aptamer region, an aptamer mask region, a junction region 1, a junction region 2, an effector region, and a terminal region,

each region being connected in the order of the junction region 2, the DNA aptamer region, the aptamer mask region, and the junction region 1 from the 5′ side of the DNA construct,

at least part of the effector region being inactivated by being hybridized with the terminal region in the absence of ligands to the DNA aptamer region, and the effector region being activated dependent on the binding of ligands to the DNA aptamer region;

wherein

4 to 7 bases at the 5′ end of the DNA aptamer region are hybridized with the aptamer mask region of 3 to 5 bases length adjacent to the 3′ side of the DNA aptamer region in the absence of ligands, to form a total of 4 to 11 hydrogen bonds between bases in the hybridized region;

the junction region 2 of 1 to 5 bases length adjacent to the 5′ side of the DNA aptamer region is hybridized with the junction region 1 adjacent to the 3′ side of the aptamer mask region in the absence of ligands, to form a total of 3 or more hydrogen bonds between bases in the hybridized region; and

the effector region is adjacent to the 3′ side of the junction region 1 and the terminal region is adjacent to the 5′ side of the junction region 2, or the effector region is adjacent to the 5′ side of the junction region 2 and the terminal region is adjacent to the 3′ side of the junction region 1.

(3) The DNA construct or the nucleic acid construct according to the above (1) or (2), wherein the aptamer mask region forms at least one bulge loop or internal loop between bases in this region and the DNA aptamer region to which the aptamer mask region hybridizes.

(4) The DNA construct or the nucleic acid construct according to any one of the above (1) to (3), wherein the junction region 1 forms at least one bulge loop or internal loop between bases in this region and the junction region 2.

(5) The DNA construct or the nucleic acid construct according to the above (4), wherein the junction region 1 and the junction region 2 are 3 bases length each.

(6) The DNA construct or the nucleic acid construct according to any one of the above (1) to (5), wherein the aptamer mask region is 4 or 5 bases length.

(7) The DNA construct or the nucleic acid construct according to the above (6), wherein the aptamer mask region forms 2 base pairs and a T-G mismatched base pair, or 3 or 4 base pairs between this region and the 3′ end of the DNA aptamer region or the 5′ end of the DNA aptamer region in the absence of ligands.

(8) The DNA construct or the nucleic acid construct according to any one of the above (1) to (7), wherein the DNA aptamer region forms hydrogen bonds between bases in this region and the aptamer mask region in the absence of ligands in 4 bases at the 3′ end of the DNA aptamer region or the 5′ end of the DNA aptamer region.

(9) The DNA construct or the nucleic acid construct according to the above (8), wherein

the aptamer mask region is T-(X)ₙ-T-T from the 5′ side and 4 bases at the 3′ end of the DNA aptamer region is A-A-Z-G from the 5′ side when the DNA aptamer region is adjacent to the 3′ side of the aptamer mask region, or

the aptamer mask region is T-T-(X)ₙ-T from the 5′ side and 4 bases at the 3′ end of the DNA aptamer region is G-Z-A-A from the 5′ side when the DNA aptamer region is adjacent to the 5′ side of the aptamer mask region; and

n is 1 or 2, and when n is 2, two (2) Xs may be the same base or different bases and (X)ₙ and Z form an internal loop or a bulge loop, or when n is 1, X and Z are selected from a combination of bases forming an internal loop between X and Z.

(10) The DNA construct or the nucleic acid construct according to any one of the above (6) to (9);

wherein

the aptamer mask region is 4 bases length; and

when the aptamer mask region has a mismatched base pair in the absence of ligands, the bases forming the mismatched base pair are selected from a combination of bases so that an increase in dG (ddG) of a secondary structure in the whole molecule due to the mismatched base pair in the aptamer mask region is +0.1 kcal/mol or more; and/or

when the junction region has a mismatched base pair in the absence of ligands, the bases forming the mismatched base pair are selected from a combination of bases so that an increase in dG of a secondary structure in the whole molecule due to the mismatched base pair in the junction region is +1.0 kcal/mol or less.

(11) The DNA construct or the nucleic acid construct according to any one of the above (1) to (10), wherein when a ligand binds to the aptamer region, part of the bases in the aptamer mask region are hybridized with the junction region 2 to form 4 or more hydrogen bonds.

(12) The DNA construct or the nucleic acid construct according to the above (11), wherein 4 or more hydrogen bonds formed between part of the bases in the aptamer mask region and the junction region 2 are formed by 2 base pairs, 2 base pairs and a T-G mismatched base pair, or 3 base pairs.

(13) The DNA construct or the nucleic acid construct according to any one of the above (1) to (12), wherein when a DNA molecule forms a secondary structure, a change in free energy (dG) in the absence of ligands is −12 to −5 (kcal/mol).

(14) The DNA construct or the nucleic acid construct according to any one of the above (1) to (13), wherein the DNA aptamer region is a patulin aptamer.

(15) The DNA construct or the nucleic acid construct according to the above (14), wherein the patulin aptamer has 80% or more sequence identity to the base sequence of SEQ ID NO: 24, SEQ ID NO: 25, or SEQ ID NO: 26.

(16) The DNA construct or the nucleic acid construct according to the above (14), wherein the patulin aptamer has the base sequence of SEQ ID NO: 24, SEQ ID NO: 25, or SEQ ID NO: 26 wherein 1 to 5 bases at the end of this base sequence may be deleted.

(17) The DNA construct or the nucleic acid construct according to any one of the above (1) to (16), wherein the effector region is a signal-generating region that is activated dependent on the ligands to the DNA aptamer region wherein measurement of the enzymatic activity of the signal-generating region enables the detection or determination of ligands.

(18) The DNA construct or the nucleic acid construct according to any one of the above (1) to (17), wherein the effector region can exert 2-fold higher activity than that in the absence of ligands by being activated dependent on the binding of ligands to the DNA aptamer region.

(19) The DNA construct or the nucleic acid construct according to the above (17) or (18), wherein the effector region is a DNAzyme.

(20) The DNA construct or the nucleic acid construct according to the above (19), wherein the DNAzyme is a redox DNAzyme having the base sequence of SEQ ID NO: 16.

(21) The DNA construct or the nucleic acid construct according to the above (20), wherein the base sequence is the base sequence of SEQ ID NO: 21, SEQ ID NO: 22, SEQ ID NO: 23, SEQ ID NO: 40, or SEQ ID NO: 41

(22) A method for detecting a ligand using the DNA construct or the nucleic acid construct according to any one of the above (19) to (21), the method including detecting the binding of the DNA construct or the nucleic acid construct with the ligand as a change in the absorbance generated by the oxidation of reduced ABTS.

(23) A sensor element including an electrode, wherein the DNA construct or the nucleic acid construct according to any one of the above (1) to (21) is immobilized on a surface of the electrode.

(24) The sensor element according to the above (23), wherein the DNA construct or the nucleic acid construct is immobilized on the surface of the electrode via a linker.

(25) A microarray including the sensor element according to the above (23) or (24).

(26) A method for detecting a ligand using the sensor element according to the above (23) or (24) or using the microarray according to the above (25), the method including measuring electrical signals in the presence of the substrate of a DNAzyme.

(27) A method for screening for a DNA molecule for detection of a ligand or a nucleic acid molecule having a base sequence equivalent thereto, the method including the following steps of:

(A) obtaining a DNA molecule candidate group for detection of a ligand or a nucleic acid molecule having a base sequence equivalent thereto by designing or modifying the base sequence of a DNA molecule, which is composed of a DNA aptamer region, an effector region that is activated on ligand-binding, an aptamer mask region, a junction region 1, and a junction region 2, includes a module region that intervenes between the DNA aptamer region and the effector region, and also forms a loop structure in the absence of the ligand,

(B) fabricating a microarray equipped with a sensor element in which a DNA molecule or a nucleic acid molecule having the designed or modified base sequence is immobilized on the electrode surface,

(C) electrochemically measuring the redox current from the effector region using the obtained microarray, and

(D) selecting a DNA molecule or a nucleic acid molecule using the detection sensitivity of a ligand as an index.

(28) The screening method according to the above (27), which is used for optimization of the detection sensitivity of a ligand by a DNA molecule or a nucleic acid molecule.

(29) The method according to the above (28), wherein at least one region selected from the DNA aptamer region, the module region, the effector region, and other region(s) is selected and the base sequence is designed or modified.

(30) The method according to any one of the above (27) to (29), wherein the base sequence of a DNA molecule candidate group is obtained using the DNA construct according to any one of the above (1) to (21) as an index.

(31) The method according to any one of the above (27) to (30), wherein a ligand is patulin.

(32) The method according to any one of the above (27) to (31), which further includes, after performing screening including the steps (A), (B), (C), and (D) defined in any one of the above (27) to (31), at least one screening step including the following steps of:

(A′) obtaining a DNA molecule candidate group for detection of ligands or a nucleic acid molecule having a base sequence equivalent thereto by modifying the DNA molecule obtained by the screening performed before,

(B) fabricating a microarray equipped with a sensor element in which a DNA molecule or a nucleic acid molecule having the designed or modified base sequence is immobilized on the electrode surface,

(C) electrochemically measuring the redox current from the effector region using the obtained microarray, and

(D) selecting a DNA molecule or a nucleic acid molecule using the detection sensitivity of a ligand as an index.

(33) A DNA molecule showing patulin-binding specificity, or a nucleic acid molecule having a base sequence equivalent thereto.

(34) The DNA molecule or the nucleic acid molecule according to the above (33), which has 80% or more sequence identity to the base sequence of SEQ ID NO: 24, SEQ ID NO: 25, or SEQ ID NO: 26.

(35) The DNA molecule or the nucleic acid molecule according to the above (33), which has the base sequence of SEQ ID NO: 24, SEQ ID NO: 25, or SEQ ID NO: 26 wherein 1 to 5 bases at the end of the base sequence may be deleted.

(36) A DNA construct or a nucleic acid construct having a base sequence equivalent thereto, including a patulin aptamer region composed of the DNA molecule according to any one of the above (33) to (35) as a DNA aptamer region, and an effector region which can be activated by binding of patulin to the aptamer region.

(37) An RNA molecule showing patulin-binding specificity, or a nucleic acid molecule having a base sequence equivalent thereto.

(38) The RNA molecule or the nucleic acid molecule according to the above (37), which has 80% or more sequence identity to the base sequence of SEQ ID NO: 33, SEQ ID NO: 34, or SEQ ID NO: 35.

(39) The RNA molecule or the nucleic acid molecule a according to the above (38), which has the base sequence of SEQ ID NO: 33, SEQ ID NO: 34, or SEQ ID NO: 35 wherein 1 to 5 bases at the 5′ end of the base sequence may be deleted.

(40) An RNA construct or a nucleic acid construct having a base sequence equivalent thereto, including a patulin RNA aptamer region and a self-cleaving ribozyme, wherein the base sequence of the patulin RNA aptamer region is the base sequence of the RNA molecule according to any one of the above (37) to (39).

(41) The RNA construct or the nucleic acid construct according to the above (40), wherein the base sequence is the base sequence of SEQ ID NO: 30, SEQ ID NO: 31, or SEQ ID NO: 32.

(42) A DNA molecule encoding the RNA molecule according to any one of the above (37) to (39) or the RNA construct according to the above (40) or (41).

(43) A method for detecting patulin using the DNA molecule or the nucleic acid molecule according to any one of the above (33) to (35), the RNA molecule or the nucleic acid molecule according to any one of the above (37) to (39), the DNA construct or the nucleic acid construct according to any one of the above (1) to (21), or the RNA construct or the nucleic acid construct according to the above (40) or (41).

(44) A method for removing patulin in a sample, which includes making patulin bind to the DNA molecule or the nucleic acid molecule according to any one of the above (33) to (35), the RNA molecule or the nucleic acid molecule according to any one of the above (37) to (39), the DNA construct or the nucleic acid construct according to any one of the above (1) to (21), or the RNA construct or the nucleic acid construct according to the above (40) or (41).

(45) A column on which the DNA molecule or the nucleic acid molecule according to any one of the above (33) to (35), the RNA molecule or the nucleic acid molecule according to any one of the above (37) to (39), the DNA construct or the nucleic acid construct according to any one of the above (1) to (21), or the RNA construct or the nucleic acid construct according to the above (40) or (41) is immobilized.

A DNA construct of the present invention is advantageous in that it can highly sensitively detect ligands (e.g., patulin) electrochemically, simply, and rapidly. A screening method of the present invention is advantageous in that it can simply screen a highly sensitive DNA construct. The screening method of the present invention is also useful for the optimization of a highly sensitive DNA construct. A nucleic acid molecule of the present invention and a nucleic acid construct of the present invention are advantageous in that they specifically bind to ligands (e.g., patulin). The nucleic acid molecule of the present invention and the nucleic acid construct of the present invention are also advantageous in that they can be used for detection or removal of ligands (e.g., patulin). Especially, the DNA construct of the present invention is further advantageous in that it can electrochemically, simply, and rapidly detect ligands (e.g., patulin).

### BRIEF DESCRIPTION OF THE DRAWINGS

FIG. 1 is a schematic diagram showing an example of the secondary structure formed by a DNA construct of the present invention in the absence of ligands. The DNA construct shown in FIG. 1 is a DNA construct having the sequence of a redox DNAzyme as an effector region and having a DNA aptamer region, and the DNA construct is a single-stranded DNA but forms a secondary structure of hairpin loop structure in aqueous solution (i.e., hairpin-loop-structured DNA construct). FIG. 1A shows an example of a DNA construct (a) (which includes SEQ ID NOS: 42 and 43) of the present invention, FIG. 1B shows an example of a DNA construct (c) (which includes SEQ ID NOS: 42 and 43) of the present invention, FIG. 1C shows an example of a DNA construct (d) (which includes SEQ ID NOS: 44 and 43) of the present invention, and FIG. 1D shows an example of a DNA construct (b) (which includes SEQ ID NOS: 44 and 43) of the present invention. In the figures, bases are numbered, and the numbers represent the position of each base when the junction region is 3 bases length and the aptamer mask region is 4 bases length. In FIGS. 1A to D, the terminal region is expediently represented in 3 bases length. FIGS. 1E to H represent DNA constructs (which include SEQ ID NOS: 42, 45 and 46; SEQ ID NOS: 42, 46 and 47; SEQ ID NOS: 44, 45 and 46; and SEQ ID NOS: 44, 46 and 47, respectively) when the terminal region is 4 bases length, and each corresponds to FIGS. 1A to D. In FIGS. 1E to H, dT of 14 bases length as a linker is added to the terminal region of the DNA construct.

FIG. 2 is a schematic diagram showing an example of the secondary structure formed by a DNA construct of the present invention in the absence of ligands. The DNA construct shown in FIG. 2 is a DNA construct having the sequence of a redox DNAzyme as an effector region and having a patulin aptamer region, and the DNA construct is a single-stranded DNA but forms a secondary structure of hairpin loop structure in aqueous solution (i.e., hairpin-loop-structured DNA construct). FIG. 2A shows an example of a DNA construct (a) (which includes SEQ ID NOS: 48 and 43) of the present invention, FIG. 2B shows an example of a DNA construct (c) (which includes SEQ ID NOS: 48 and 49) of the present invention, FIG. 2C shows an example of a DNA construct (d) (which includes SEQ ID NOS: 48 and 43) of the present invention, and FIG. 2D shows an example of a DNA construct (b) (which includes SEQ ID NOS: 48 and 49) of the present invention. In the figures, bases are numbered, and the numbers represent the position of each base when the junction region is 3 bases length and the aptamer mask region is 4 bases length. In FIGS. 2A to D, the terminal region is expediently represented in 3 bases length. FIGS. 2E to H represent DNA constructs (which include SEQ ID NOS: 48, 45 and 46; SEQ ID NOS: 48, 46 and 47; SEQ ID NOS: 48, 45 and 46; and SEQ ID NOS: 48, 46 and 47, respectively) when the terminal region is 4 bases length, and each corresponds to FIGS. 2A to D. In FIGS. 2E to H, dT of 14 bases length as a linker is added to the terminal region of the DNA construct.

FIG. 3 is a view showing that the activity of a redox DNAzyme immobilized on the electrode surface of a microarray can be electrochemically detected.

FIG. 4 is a schematic diagram showing the state of a DNA construct (which includes SEQ ID NO: 50) of the present invention when the DNA construct of the present invention is synthesized on the electrode surface and is immobilized on the electrode surface. In the figure, L represents a ligand.

FIG. 5 is a view showing the outline of the redox reaction by the redox DNAzyme used in Examples after Example A2. The redox DNAzyme portion contained in a DNA construct of the present invention forms the conformation like that in the left of FIG. 5 and reduces H₂O₂ with heroin. At this time, when ABTS is added, ABTS is converted from the reduced form to the oxidized form by the effects of the DNAzyme, and absorbance is generated at 414 nm (right of FIG. 5).

FIG. 6 is a view showing the high reproducibility of the electrochemical detection method. A signal ratio (AMP concentration 5 mM/AMP concentration 0 mM) by the electrochemical detection method is plotted on the graph. In FIG. 6, the horizontal axis was defined as the first experiment and the longitudinal axis was defined as the second experiment. The black dots in the figure represent a signal ratio shown by the DNA groups subjected to screening, and the gray dots in the figure represent a signal ratio shown by the DNA aptamer construct of SEQ ID NO: 20 reported.

FIG. 7 is a view showing the effects of the length of each region of a DNA construct, the number of loops formed within the DNA construct, and the free energy (dG) when the DNA construct forms a secondary structure on the sensitivity of the DNA construct as a sensor.

FIG. 8 is the results of the replication study by absorbance measurement of ABTS on 6 DNA constructs (TMP-1 to 6), which were determined to be highly sensitive by the primary screening. The solid lines in the graphs represent the absorbance (λₘₐₓ=414 nm) of each DNA construct, and the dotted lines in FIGS. 8A, E, and F represent the absorbance observed in the DNA construct having SEQ ID NO: 20 reported.

FIG. 9 is a view showing the secondary structure of the aptamer mask region and the junction region in the stem portion formed by each DNA construct of TMP 1 to 6 in the absence of ligands. The dG described in each figure represents the free energy (dG) when the whole DNA construct forms the secondary structure.

FIG. 10 is a view showing the results of absorbance measurement of 7 DNA constructs in which the aptamer mask region was T(X)ₙTT (n is 1 or 2) from the 5′ side, among 55 constructs that showed a signal ratio higher than that of TMP-5 in the secondary screening. The immediate right of each graph shows the secondary structure of the aptamer mask region and the junction region, and dG represents the free energy (dG) when the whole DNA construct forms the secondary structure.

FIG. 11 is a view showing the results of absorbance measurement of 6 constructs that showed a good ligand-dependent DNAzyme activity, in the remaining 48 constructs among 55 constructs that showed a signal ratio higher than that of TMP-5 in the secondary screening.

FIG. 12 is a view showing the results of absorbance measurement of 24 constructs that showed no activity, in the remaining 48 constructs among 55 constructs that showed a signal ratio higher than that of TMP-5 in the secondary screening.

FIG. 13 is a view showing the effects of the type of mismatched base pair on the ligand-dependent DNAzyme activity of a DNA construct. In FIG. 13, the longitudinal axis in the graph represents the effects of mismatch on dG (ddG). In FIG. 13, as a result of absorbance measurement, the value of ddG was compared between a sequence that was highly sensitive to ligands (highly sensitive sequence) and a sequence that was low sensitive to ligands (low sensitive sequence). FIGS. 13A and B show the result when the aptamer mask region is 4 bases length, and FIGS. 13C and D show the result when the aptamer mask region is 5 bases length. FIGS. 13A and C show ddG by mismatched base pair in the aptamer mask region, and FIGS. 13B and D show ddG by mismatched base pair in the junction region.

FIG. 14 is a view showing the relationship between the results of absorbance measurement of the sequences obtained by modifying TMP-5 (TMP 5-1 to 5 and TG1) and the secondary structure after the binding of ligands. FIGS. 14A to E show the results of absorbance measurement of the sequences (which include SEQ ID NOS: 51 and 52; SEQ ID NO: 53; SEQ ID NO: 54; SEQ ID NO: 55; and SEQ ID NO: 56, respectively) that showed high sensitivity. FIGS. 14F and G are views on the DNA constructs (which include SEQ ID NO: 57 and SEQ ID NO: 58, respectively) that showed no high sensitivity. The arrows in FIG. 14A to G show where base pairs are formed within the secondary structure after the binding of ligands. The terms of bulge loop and internal loop in FIGS. 14F and G mean that each of a bulge loop and an internal loop is formed in the secondary structure after the binding of ligands. FIG. 14H (SEQ ID NO: 59) is a view showing the conformation formed by the DNA aptamer region after the binding of ligands. FIG. 14H shows that the aptamer mask region can be hybridized with the junction region 2 after the binding of ligands.

FIG. 15A is a view showing a DNA molecule, which includes SEQ ID NO: 60, in which an arginine aptamer of SEQ ID NO: 18 was used as a DNA aptamer region. The arrows in FIG. 15A show where a base pair is formed within the secondary structure after the binding of ligands. FIG. 15B is a view showing the secondary structure of the aptamer mask region and the junction region of the DNA molecule used as a control in the absence of ligands. FIG. 15C is a view showing the results of measurement of the oxidation activity of ABTS for arginine of each molecule of TMP-5^{Arg} (solid line) and control (dotted line).

FIG. 16 is an example of the patulin aptamer fabricated by modifying the DNA aptamer region of the TMP-5 molecule (FIG. 16A: a DNA construct having the base sequence of SEQ ID NO: 23), and a view showing the electrical signal ratio on molecules with an electrical signal ratio being 2-fold or more higher among the molecules screened with an electrochemical detection microarray (FIGS. 16B and C).

FIG. 17 is a view showing the colorimetric test results on 6 sequences with mean electrical signal ratio at a patulin concentration of 5 mM being 4 or more among the sequences shown in FIG. 16B as a sequence with an electrical signal ratio being 2-fold or more higher (candidate group 1-1 to 1-6), and 12 sequences with mean electrical signal ratio at a patulin concentration of 100 μM being 3 or more or with mean electrical signal ratio at a patulin concentration of 5 mM being 2 or more among the sequences shown in FIG. 14C as a sequence with an electrical signal ratio being 2-fold or more higher (candidate group 2-1 to 2-12).

FIG. 18 is a view showing that the colorimetric test result on the candidate group 2-7 can be improved at low temperature.

FIG. 19 is a view showing the results of estimation of the secondary structure of the patulin aptamer region for the candidate groups 1-5 (which include SEQ ID NO: 61) and 1-6 (which include SEQ ID NO: 62) and the candidate groups 2-7 (which include SEQ ID NO: 63).

FIG. 20 is a view showing that the candidate group 2-7 has binding specificity for patulin.

FIG. 21 is a view showing the results of estimation of the secondary structure of the self-cleaving ribozyme used in the screening for the patulin RNA aptamer (FIG. 21A (SEQ ID NO: 64), and of the secondary structure of 3 types of patulin RNA aptamer regions obtained by the screening (FIG. 21B) (SEQ ID NOs: 33, 34 and 35). N₃₅ in FIG. 21A_means that 35 bases of N (each N is independently selected from any of A, U, G, or C) are arranged in the sequence.

FIG. 22 is a view showing the results of detection of self-cleavage in the self-cleaving ribozyme. FIG. 22A shows the results of electrophoresis of the RNA construct of SEQ ID NO: 30 obtained by screening and its fragments by self-cleavage, by modified PAGE using 8M urea, and FIG. 22B shows the cleaving activity (relative value) of 3 types of RNA constructs obtained by screening. In other words, in FIG. 22B, the amount of cleaved RNA molecules in the presence of patulin is relatively shown, using the amount of RNA molecules cleaved in the absence of patulin as 1.

FIG. 23 is a view showing the results of detection of patulin by the RNA construct having the base sequence of SEQ ID NO: 30. In FIG. 23, the amount of cleaved RNA molecules in the presence of patulin is relatively shown, using the amount of RNA molecules cleaved in the absence of patulin as 1.

FIG. 24 is a view showing that the RNA construct having the base sequence of SEQ ID NO: 30 has binding specificity for patulin.

FIG. 25 is a view showing the change in cleaving activity when 4 bases at the 5′ end or the 3′ end of the patulin aptamer portion of the RNA construct having the base sequence of SEQ ID NOS: 31 and 32 were deleted. The relative intensity of cleaving activity (longitudinal axis) represents a ratio of the cleaving activity after addition of patulin to the cleaving activity before addition of patulin.

FIG. 26 is a view showing the results of detection of patulin by the modified DNA construct (SC-7-CCCA) of the candidate group 2-7 (SC-7) having a patulin aptamer region.

FIG. 27 is a view showing the results of detection of AMP in TMP-5-CCCA obtained by adding A to the 3′ end of TMP-5 (SEQ ID NO: 2).

FIG. 28 is a view showing the results of detection of patulin in apple juice using SC-7-CCCA.

FIG. 29 is a view showing the results of detection of patulin by SC-7-CCCA-TMP-7 (left of FIG. 29), and the secondary structure of the TMP-7 region (right of FIG. 29).

### DETAILED DESCRIPTION OF THE INVENTION

As used herein, a “nucleic acid” means natural nucleic acids such as DNA and RNA, and nucleic acid mimics such as artificial nucleic acids including 7-(2-thienyl)imidazo[4,5-b]pyridine (Ds), 2-nitro-4-propynylpyrrole (Px), peptide nucleic acid (PNA), and locked nucleic acid (LNA). As used herein, a “nucleic acid molecule” means a molecule constituted of any one of the selected groups constituted of nucleic acids such as DNA and RNA, and nucleic acid mimics such as 7-(2-thienyl)imidazo[4,5-b]pyridine (Ds), 2-nitro-4-propynylpyrrole (Px), peptide nucleic acid (PNA), and locked nucleic acid (LNA), or a molecule constituted of the hybrid between the nucleic acid and nucleic acid mimics. PNA means a nucleic acid having a skeleton in which N-(2-aminoethyl)glycine is bound via an amide bond, instead of sugars constituting the main chain of DNA and RNA, and LNA means a nucleic acid having a cyclic structure in which the oxygen atom at the 2′ position and the carbon atom at the 4′ position of the ribonucleic acid constituting the main chain are crosslinked via methylene. A nucleic acid molecule constituted of these PNA and LNA has only a difference in the main chain skeleton of the nucleic acid, and the base portion can be one having bases equivalent to those in DNA or RNA; when PNA or LNA has bases equivalent to those in DNA or RNA, the stability as a nucleic acid molecule can be improved while maintaining the nature of the base portion related to base pair formation. RNA, PNA, or LNA having bases equivalent to those in DNA means, when a base in DNA is A, T, G, or C, RNA having a base of A, U, G, or C, respectively, and PNA and LNA having a base of A, T (or U), G, or C, respectively. DNA, PNA, or LNA having bases equivalent to those in RNA means, when a base in RNA is A, U, G, or C, DNA having a base of A, T, G, or C, and PNA and LNA having a base of A, T (or U), G, or C. Bases in a nucleic acid molecule or a nucleic acid construct may be modified or not. As a modified base, for example, a modified base by molecular labeling such as fluorescent molecules including 2-aminopurine and fluorescein has been known, and persons skilled in the art can appropriately perform various modifications to a nucleic acid molecule or a nucleic acid construct.

As used herein, a “nucleic acid molecule having a base sequence equivalent to that in DNA molecule” is preferably a hybrid-type nucleic acid molecule in which part of a DNA molecule showing binding specificity for patulin as mentioned later, for example, bases of 50% or less, 40% or less, 30% or less, 20% or less, 10% or less, 5% or less, or 3% or less of the sequence, or 1 or 2 base(s), is constituted of a nucleic acid other than DNA which has a base sequence equivalent to the part, and having a function equivalent to that of a DNA molecule of the present invention. As used herein, a “nucleic acid molecule having a base sequence equivalent to that in RNA molecule” is preferably a hybrid-type nucleic acid molecule in which part of an RNA molecule showing binding specificity for patulin as mentioned later, for example, bases of 50% or less, 40% or less, 30% or less, 20% or less, 10% or less, 5% or less, or 3% or less of the sequence, or 1 or 2 base(s), is constituted of a nucleic acid other than RNA which has a base sequence equivalent to the part, and having a function equivalent to that of an RNA molecule of the present invention. Therefore, in the present description, a “nucleic acid molecule having a base sequence equivalent to that in DNA molecule” has a base sequence equivalent to that in a DNA molecule of the present invention as mentioned above, and is constituted of DNA and nucleic acids other than DNA (e.g., RNA, PNA, or LNA). A “nucleic acid molecule having a base sequence equivalent to that in RNA molecule” has a base sequence equivalent to that in an RNA molecule of the present invention as mentioned above, and is constituted of RNA and nucleic acids other than RNA (e.g., DNA, PNA, or LNA). Whether a “nucleic acid molecule having a base sequence equivalent to that in DNA molecule” has a function equivalent to that of the DNA molecule or not can be evaluated, for example, by detecting an intermolecular bond by surface plasmon resonance as mentioned later. Whether a “nucleic acid molecule having a base sequence equivalent to that in RNA molecule” has a function equivalent to that of the RNA molecule or not can be evaluated, for example, by detecting an intermolecular bond by surface plasmon resonance as mentioned later.

As used herein, a “nucleic acid construct having a base sequence equivalent to that in DNA construct” is a hybrid-type nucleic acid construct in which part of the DNA construct as mentioned later, for example, bases of 50% or less, 40% or less, 30% or less, 20% or less, 10% or less, 5% or less, or 3% or less of the sequence, or 1 or 2 base(s), is constituted of a nucleic acid other than DNA which has a base sequence equivalent to the part. As used herein, a “nucleic acid construct having a base sequence equivalent to that in RNA construct” is a hybrid-type nucleic acid construct in which part of the RNA construct as mentioned later, for example, bases of 50% or less, 40% or less, 30% or less, 20% or less, 10% or less, 5% or less, or 3% or less of the sequence, or 1 or 2 base(s), is constituted of a nucleic acid other than RNA which has a base sequence equivalent to the part. Therefore, in the present description, a “nucleic acid construct having a base sequence equivalent to that in DNA construct” has a base sequence equivalent to that in a DNA construct of the present invention as mentioned above, is constituted of DNA and nucleic acids other than DNA, and has a function equivalent to that of a DNA construct of the present invention. A “nucleic acid construct having a base sequence equivalent to that in RNA construct” has a base sequence equivalent to that in an RNA construct of the present invention as mentioned above, is constituted of RNA and nucleic acids other than RNA, and has a function equivalent to that of an RNA construct of the present invention. Especially, when the effector region of a DNA construct of the present invention as mentioned later is ligand-dependently hybridized with other nucleic acid molecules, by replacing the DNA in the effector region with PNA or LNA, the specificity when the effector region is hybridized with other nucleic acid molecules can be improved. The linker portion of a DNA molecule immobilized on a sensor element of the present invention as mentioned later can be replaced by PNA or LNA, thereby enabling improvement in the chemical stability of a nucleic acid molecule. Whether a “nucleic acid construct having a base sequence equivalent to that in DNA construct” has a function equivalent to that of the DNA construct or not can be evaluated with the ligand-dependent activation of the effector region as mentioned later as an index, and can be evaluated, for example, in accordance with the procedure for the electrochemical detection method or the colorimetric test using ABTS mentioned in Example A3. Whether a “nucleic acid construct having a base sequence equivalent to that in RNA construct” has a function equivalent to that of the RNA construct or not can be evaluated, for example, using the ligand-dependent self-cleaving activity as an index, and can be evaluated, for example, in accordance with the procedure mentioned in Example C1.

Into or to the above mentioned a “nucleic acid molecule having a base sequence equivalent to that in DNA molecule,” a “nucleic acid molecule having a base sequence equivalent to that in RNA molecule,” a “nucleic acid construct having a base sequence equivalent to that in DNA construct,” and a “nucleic acid construct having a base sequence equivalent to that in RNA construct,” an artificial nucleic acid (e.g., Ds and Px) of 1 to 10 bases, preferably 1, 2, or 3 base(s) may be inserted or added, provided that the function of these nucleic acid molecules and nucleic acid constructs is maintained. In the above mentioned a “nucleic acid molecule having a base sequence equivalent to that in DNA molecule,” a “nucleic acid molecule having a base sequence equivalent to that in RNA molecule,” a “nucleic acid construct having a base sequence equivalent to that in DNA construct,” and a “nucleic acid construct having a base sequence equivalent to that in RNA construct,” provided that the function of these nucleic acid molecules and nucleic acid constructs is maintained, compounds forming an artificial base pair to each other such as Ds and Px can also be introduced into a nucleic acid molecule, and a base pair in a nucleic acid molecule or a nucleic acid construct can also be substituted by an artificial base pair of Ds-Px as mentioned above.

As used herein, “binding” means having a property of binding to ligands, and “showing binding specificity” means having a property of specifically binding to ligands. Therefore, for example, “patulin-binding” means having a property of binding to patulin, and “patulin-binding specificity” means having a property of specifically binding to patulin.

As used herein, a “ligand” includes, but not limited to, for example, adenosine monophosphate (AMP), patulin, and arginine, etc. and is preferably patulin.

Patulin is a compound having the chemical structure represented by the following formula:

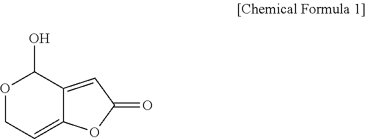 [SMILES: O=C1C=C2C(=CCOC2O)O1]

Patulin is a type of mycotoxin secreted from molds such as Penicillium or Aspergillus, and is known to be generally detected from rotten apples, grapes, or peaches, etc.

As used herein, an “aptamer” means a nucleic acid molecule showing binding specificity for ligands or part of the nucleic acid molecule, and DNA (or RNA) showing binding specificity for ligands can be referred to as “DNA aptamer” (or “RNA aptamer”). Especially, when a ligand is patulin, “aptamer” can be referred to as “patulin aptamer,” and especially when an aptamer is DNA (or RNA), “aptamer” can be referred to as “patulin DNA aptamer (or patulin RNA aptamer).”

Values of the identity of base sequence can be calculated in accordance with a well-known algorithm, and, for example, can be calculated with default parameters using BLAST (http://www.ddbj.nig.ac.jp/search/blast-j.html).

As used herein, “hybridize” means that certain polynucleotide forms a double strand via a hydrogen bond of bases of the polynucleotide and complementary to the target polynucleotide. Hybridization can be performed under a stringent condition. A “stringent condition” can be determined dependent on the Tm (° C.) of the double strand between the primer sequence and its complementary strand and necessary salt concentrations, etc., and setting an appropriate stringent condition after a sequence to be a probe is selected is a well-known technique for persons skilled in the art (e.g., see J. Sambrook, E. F. Frisch, T. Maniatis; Molecular Cloning 2nd edition, Cold Spring Harbor Laboratory (1989), etc.). A stringent condition includes, for example, performing hybridization reaction in an appropriate buffer usually used for hybridization and at a temperature slightly lower than the Tm determined by nucleotide sequence (e.g., temperature lower than Tm by 0 to about 5° C.). A stringent condition also includes, for example, washing after hybridization reaction in a high-concentration and low-salt concentration solution. An example of a stringent condition includes a washing condition in a 6×SSC/0.05% sodium pyrophosphate solution.

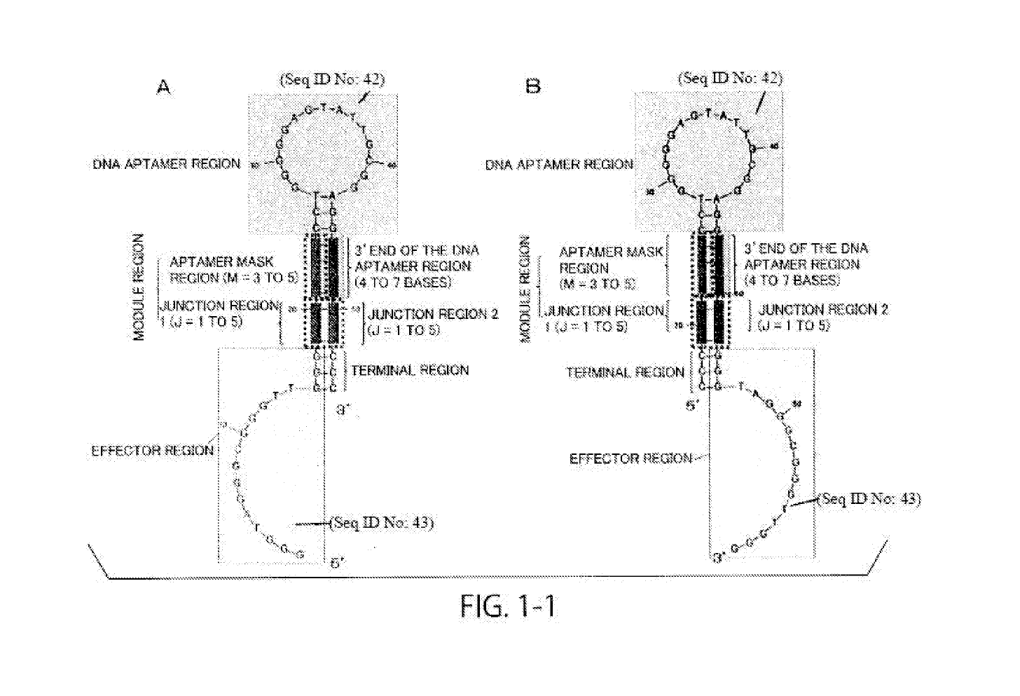

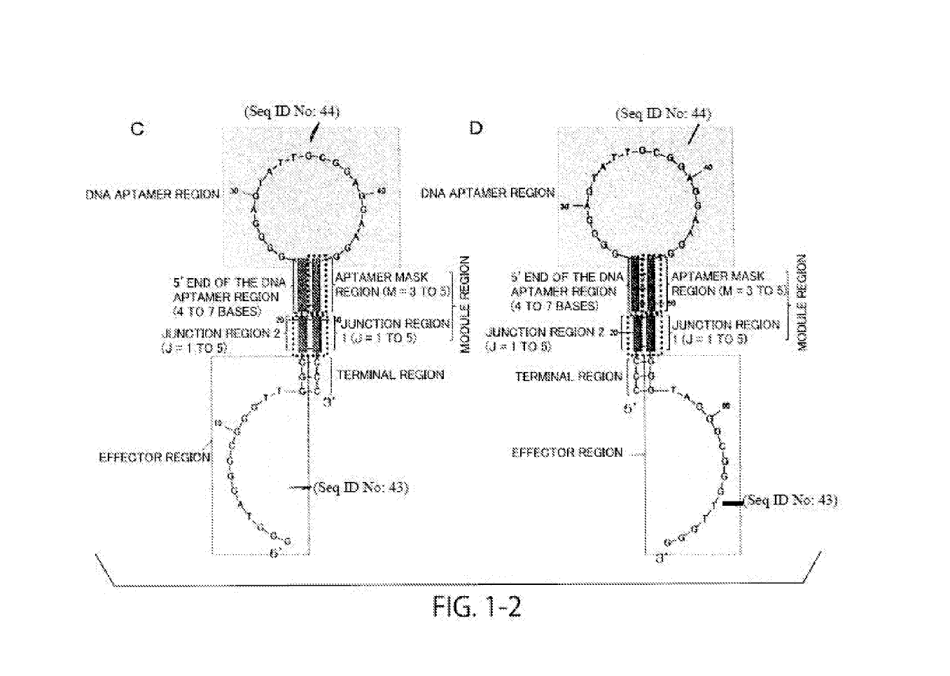

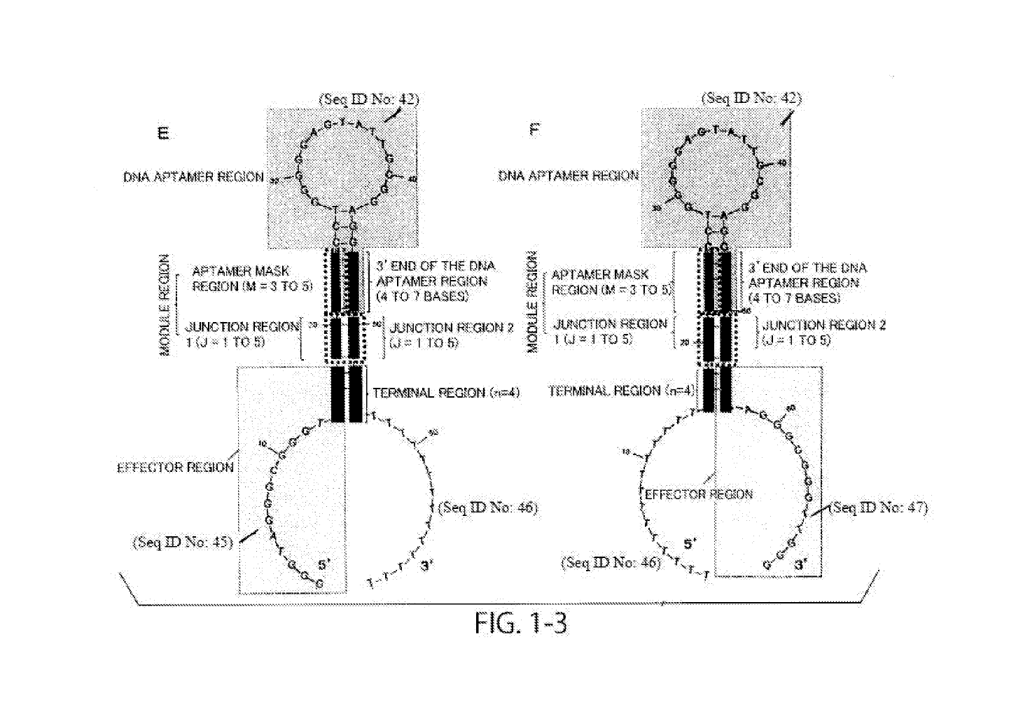

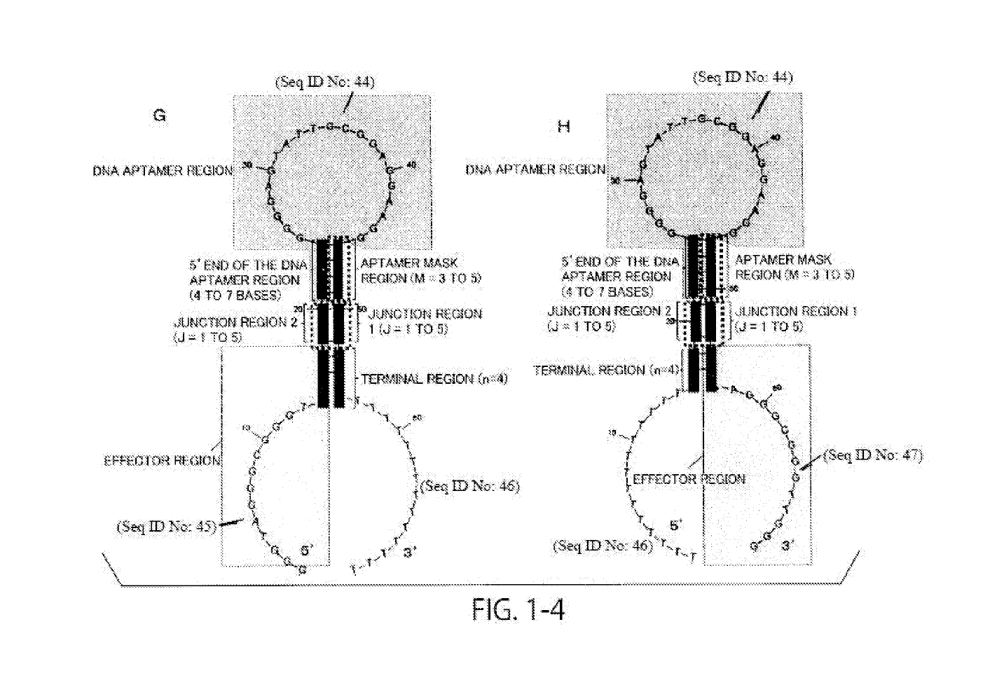

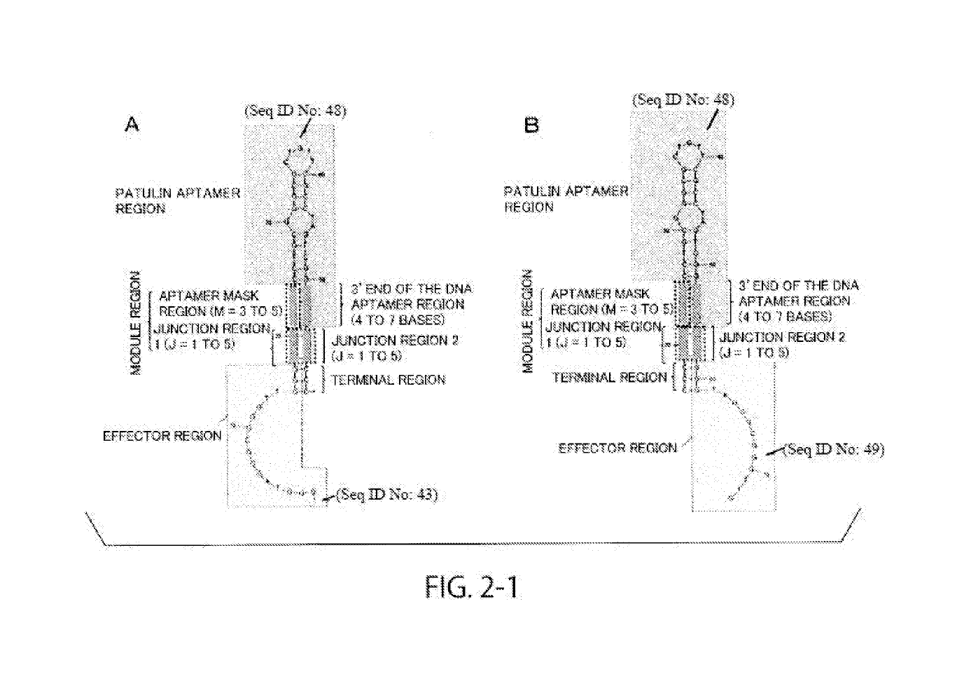

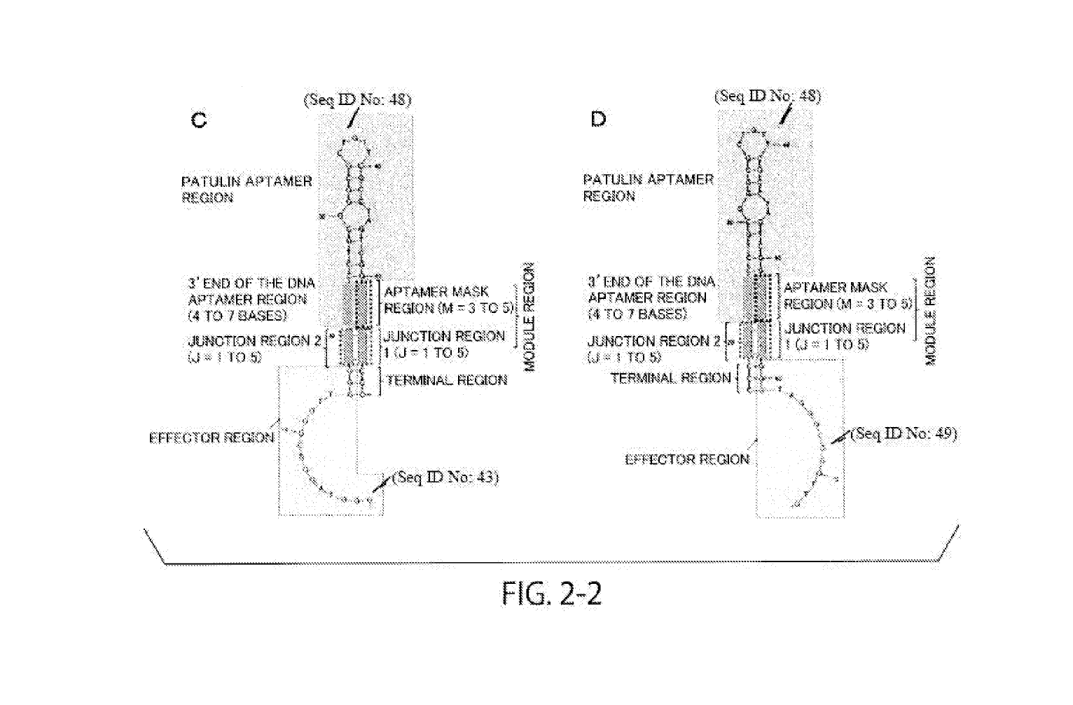

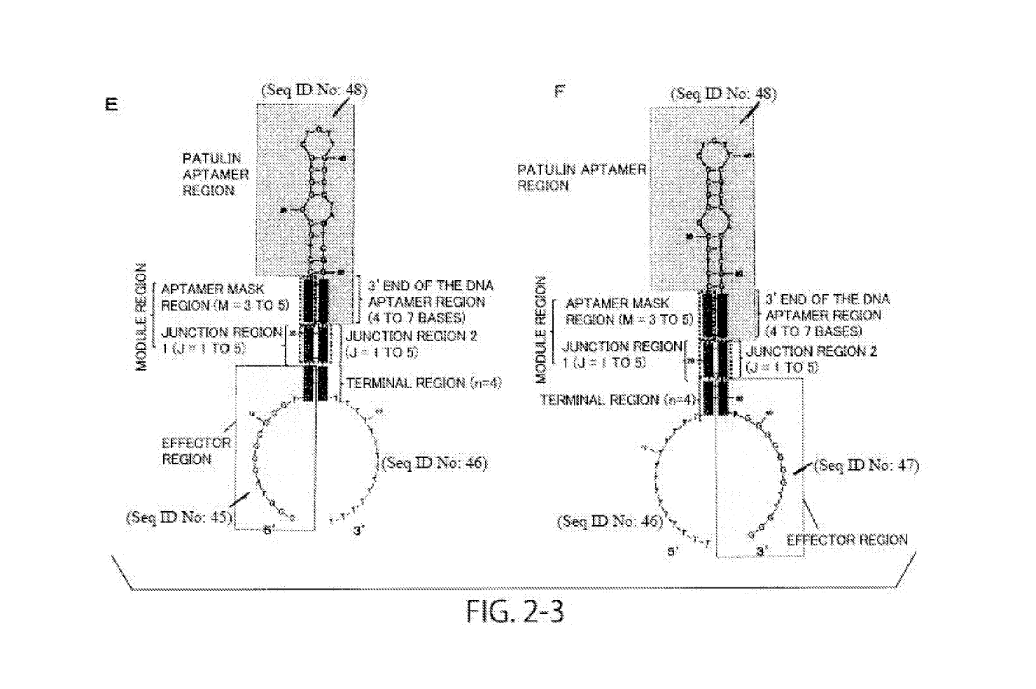

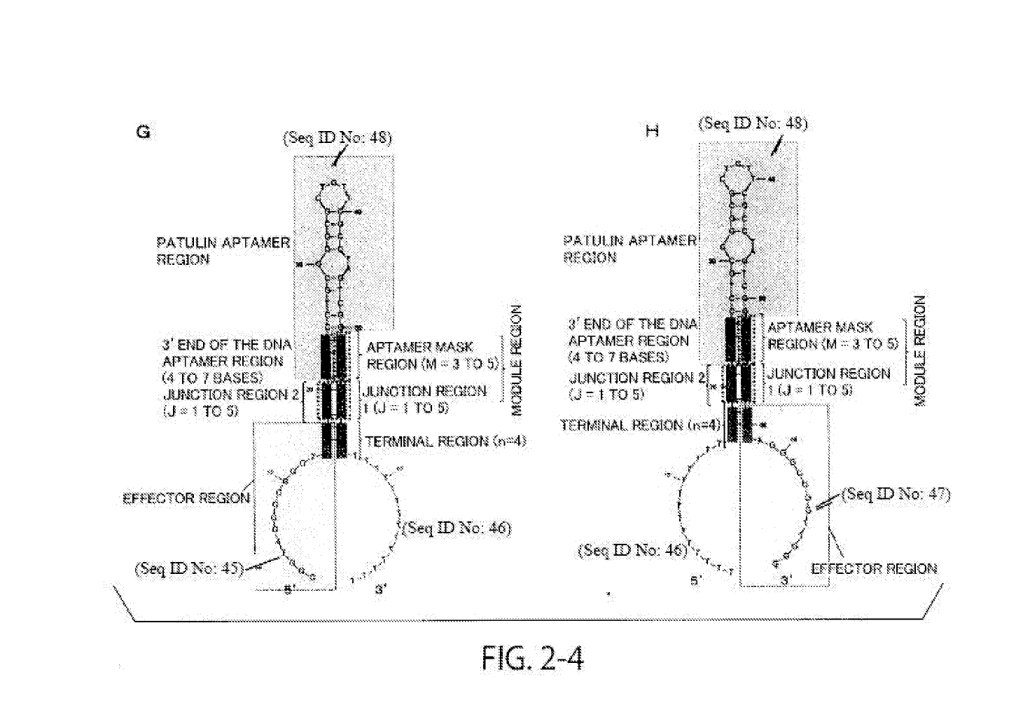

A DNA construct of the present invention will be described below based on FIGS. 1 and 2.

A DNA construct of the present invention is a DNA construct forming a loop structure, including a patulin aptamer region and an effector region, and, specifically, can be a DNA construct constituted of

(i) effector region,
(ii) junction region 1,
(iii) aptamer mask region,
(iv) DNA aptamer region,
(v) junction region 2, and
(vi) terminal region
(hereinafter referred to as a DNA construct of the present invention). Such DNA construct can be used for detection of ligands via the effector region. Therefore, in the present invention, a DNA construct used for detection of ligands, i.e., a DNA sensor molecule, is provided.

A DNA construct of the present invention will be described in detail below with reference to FIGS. 1 and 2.

In a DNA construct of the present invention, the effector region is inactivated by being masked by the terminal region within the DNA molecule in the absence of a ligand to the DNA aptamer region, but when the ligand binds to the DNA aptamer region, the effector region is activated with the masking removed. In other words, a DNA molecule of the present invention is a DNA construct for detection of ligands in which the effector region is activated dependent on the binding to ligands. When the effector region is activated, for example, enzymatic activity is activated or it becomes able to be hybridized with other nucleic acid molecules, thereby enabling detection of ligands and ligand-dependent cell function control. In other words, a DNA construct of the present invention is a DNA construct intended for detection of ligands and ligand-dependent cell function control. Since a DNA construct of the present invention is constituted of DNA, the chemical stability is higher than that in RNA or proteins, and synthesis, handling, and storage is easy. A DNA construct of the present invention is a DNA construct forming at least one loop structure by the DNA aptamer region, and preferably a DNA construct forming one loop structure, i.e., a hairpin-loop-structured DNA construct.

A DNA construct of the present invention is preferably a DNA construct in which (i) to (vi) are connected in the above mentioned order from the 5′ end (hereinafter referred to as “DNA construct (a).” See FIGS. 1A (SEQ ID NOS: 42 and 43), 1E (SEQ ID NOS: 42, 45 and 46), 2A (SEQ ID NOS: 48 and 43), and 2E (SEQ ID NOS: 48, 45 and 46)[[.]]), but not limited to this, provided that the inactivated effector region is activated dependent on the ligand-binding.

For example, a DNA construct of the present invention may be a DNA construct in which each region is connected from the 5′ end in the following order of:

(vi) terminal region,
(v) junction region 2,
(iv) DNA aptamer region,
(iii) aptamer mask region,
(ii) junction region 1, and
(i) effector region
(hereinafter referred to as “DNA construct (b).” See FIG. 1D (SEQ ID NOS: 44 and 43), 1H (SEQ ID NOS: 44, 46 and 47), 2D (SEQ ID NOS: 48 and 49), and 2H (SEQ ID NOS: 48, 46 and 47)[[.]]).

A DNA construct of the present invention may be a DNA construct in which (i) and (vi) change places in the above DNA construct (a) and (b), i.e., a DNA construct in which each region is connected from the 5′ end in the order of:

(vi) terminal region,
(ii) junction region 1,
(iii) aptamer mask region,
(iv) DNA aptamer region,
(v) junction region 2, and
(i) effector region
(hereinafter referred to as “DNA construct (c).” See FIG. 1B (SEQ ID NOS: 42 and 43), 1F (SEQ ID NOS: 42, 46 and 47), 2B (SEQ ID NOS: 48 and 49), and 2F (SEQ ID NOS: 48, 46 and 47)[[.]]), or a DNA construct in which each region is connected from the 5′ end in the order of:
(i) effector region,
(v) junction region 2,
(iv) DNA aptamer region,
(iii) aptamer mask region,
(ii) junction region 1, and
(vi) terminal region
(hereinafter referred to as “DNA construct (d)”, see FIG. 1C (SEQ ID NOS: 44 and 43), 1G (SEQ ID NOS: 44, 45 and 46), a 2C (SEQ ID NOS: 48 and 43), and 2G (SEQ ID NOS: 44, 45 and 46)). A DNA construct of the present invention may be any one of the above mentioned DNA construct (a), (b), (c), and (d), and preferably is a DNA construct (a) or a DNA construct (b), most preferably a DNA construct (a).

In other words, a DNA construct of the present invention can be said as a DNA construct forming a loop structure, including a DNA aptamer region, an aptamer mask region, a junction region 1, a junction region 2, an effector region, and a terminal region, wherein

4 to 7 bases at the 3′ end of the DNA aptamer region are hybridized with the aptamer mask region of 3 to 5 bases length adjacent to the 5′ side of the DNA aptamer region in the absence of ligands (i.e., corresponding to a DNA construct (a) or (c)), forming a total of 4 to 11 hydrogen bonds between bases in the hybridized region, or 4 to 7 bases at the 5′ end of the DNA aptamer region are hybridized with the aptamer mask region of 3 to 5 bases length adjacent to the 3′ side of the DNA aptamer region in the absence of ligands (i.e., corresponding to a DNA construct (b) or (d)), forming a total of 4 to 11 hydrogen bonds between bases in the hybridized region,

the junction region 2 of 2 to 5 bases length adjacent to the 3′ side of the DNA aptamer region is hybridized with the junction region 1 adjacent to the 5′ side of the aptamer mask region in the absence of ligands, forming a total of 3 or more hydrogen bonds between bases in the hybridized region (when 4 to 7 bases at the 5′ end of the DNA aptamer region are hybridized with the aptamer mask region of 3 to 5 bases length adjacent to the 3′ side of the DNA aptamer region in the absence of ligands, the junction region 2 of 2 to 5 bases length adjacent to the 5′ side of the DNA aptamer region is hybridized with the junction region 1 adjacent to the 3′ side of the aptamer mask region in the absence of ligands, forming a total of 3 or more hydrogen bonds between bases in the hybridized region),

the effector region is adjacent to the 5′ side of the junction region 1 and the terminal region is adjacent to the 3′ side of the junction region 2, or adjacent to the 3′ side of the junction region 2 and the terminal region is adjacent to the 5′ side of the junction region 1 (when 4 to 7 bases at the 5′ end of the DNA aptamer are hybridized with the aptamer mask region of 3 to 5 bases length adjacent to the 3′ side of the DNA aptamer in the absence of ligands, the effector region is adjacent to the 3′ side of the junction region 1 and the terminal region is adjacent to the 5′ side of the junction region 2, or the effector region is adjacent to the 5′ side of the junction region 2 and the terminal region is adjacent to the 3′ side of the junction region 1.), and at least part of the effector region is inactivated by being hybridized with the terminal region in the absence of ligands, and the effector region is activated dependent on the binding of ligands to the DNA aptamer region.

As used herein, an “effector region” is a signal-generating region itself having enzymatic activity, or a sequence that can be hybridized with other nucleic acid molecules. An effector region of the present invention is inactivated by being masked by the terminal region of the DNA construct when the DNA aptamer region is not bound to ligands, but when the effector region binds to ligands, it becomes in the free state (hereinafter referred to as “activation of effector region”), and it activates the enzymatic activity of the signal-generating region, or becomes able to be hybridized with other nucleic acid molecules. In the present invention, by monitoring the activation of the effector region, ligands can be detected or determined.

In one aspect of the present invention, the effector region is a signal-generating region. In this aspect, by measuring the enzymatic activity of the signal-generating region of the DNA construct, ligands can be detected or determined.

As used herein, a “signal-generating region” is a region constituted of DNA itself having enzymatic activity. As a signal-generating region, a DNAzyme can be used, and preferably, a redox DNAzyme, and more preferably, a redox DNAzyme having the sequence of SEQ ID NO: 16 can be used. The activity of the redox DNAzyme can be electrochemically detected. When a redox DNAzyme having the sequence of SEQ ID NO: 16 is used, although there is no particular limitation on the substrate, preferably, 2,2′-azino-bis(3-ethylbenzothiazoline-6-sulfonic acid) (ABTS) can be added as a substrate. Advantage of using ABTS as a substrate is in that the activity of a DNAzyme can be easily measured; in other words, when ABTS is used as a substrate, by measuring the absorbance (λₘₐₓ=414 nm) of oxidized ABTS produced by a redox DNAzyme, the activity of a DNAzyme can be simply measured. In electrochemical measurement, although not essential, addition of ABTS as a substrate is advantageous in that redox reaction is improved and thus electrochemical detection sensitivity is expected to be improved, and that results consistent with the results of a colorimetric test (absorbance measurement) tend to be obtained. In the present invention, by measuring the enzymatic activity of the signal-generating region, the binding of ligands to the DNA construct can be detected or ligands can be determined. Ligands can be determined with well-known methods for persons skilled in the art such as a method using calibration curve.

In one aspect of the present invention, a DNA construct of the present invention in which the effector region is hybridized with other nucleic acid molecules is provided. In this aspect, by measuring the amount of hybridization of the other nucleic acid molecules with the DNA construct, ligands can be detected or determined, or by hybridizing with an in vivo nucleic acid molecule, the function of the in vivo nucleic acid molecule can be regulated.

Other nucleic acid molecules with which an effector region of the present invention is hybridized can be a nucleic acid molecule to which enzymes or labels are bound, or an in vivo nucleic acid molecule. Therefore, an effector region of the present invention can be a sequence that can be hybridized with these nucleic acid molecules, preferably a DNA having a sequence complementary to that of these nucleic acid molecules.

There is no particular limitation on enzymes or labels that can be bound to other nucleic acid molecules with which an effector region of the present invention is hybridized, and for example, enzymes such as horseradish peroxidase (HRP) or alkaline phosphatase (AP), or labels such as fluorescence or RI can be used. The activation of the effector region of a DNA construct of the present invention can be monitored as the amount of a nucleic acid molecule to be hybridized by measuring the enzymatic activity such as HRP or AP, or labels such as fluorescence or radioisotope (RI), thereby enabling detection of the binding of ligands to the DNA construct or determination of ligands. Gold nanoparticles may be bound to other nucleic acid molecules with which an effector region of the present invention is hybridized. When gold nanoparticles are aggregated, the absorption spectrum is changed. For example, when gold particles is bound also to a DNA construct of the present invention and both are hybridized with each other to shorten the distance between the gold particles, interaction between a DNA construct of the present invention and other nucleic acid molecules with which the effector region is hybridized can be confirmed by utilizing the nature in which the absorption spectrum of a molecule becomes at low-wavelength side. Gold nanoparticles can be bound to a DNA construct of the present invention via thiol.

There is no particular limitation on an in vivo nucleic acid molecule with which an effector region of the present invention is hybridized, but mRNA and genomic DNA can be used. In one aspect of the present invention, the effector region of a DNA construct of the present invention is a DNA constituted of a sequence that can be hybridized with a certain mRNA molecule, and with activation of the effector region, the effector region is hybridized with the mRNA and inhibits translation of proteins from the mRNA. Such certain mRNA includes, for example, mRNA molecules such as matrix metalloproteinase (MMP) overexpressed in cancer cells, and some of them are publicly known (Liotta L. A., Tryggvason K., Garbisa S., Hart I., Foltz C. M., Shafie S., Metastatic potential correlates with enzymatic degradation of basement membrane collagen. (1980) Nature, 284:67-68). In other aspects of the present invention, the effector region of a DNA construct of the present invention can be a DNA constituted of a sequence that can be hybridized with the telomeric region of a genome, and in this case, with the activation of the effector region, the effector region inhibits the expansion of a telomere by telomerase by forming a G-quadruplex structure together with the telomeric region. In Deng M., Zhang D., Zhou Y., Zhou X., Highly effective colorimetric and visual detection of nucleic acids using an asymmetrically split peroxidase DNAzyme. (2008) J. Am. Chem. Soc., 130 (39):13095-102, it is stated that a G-quadruplex structure is formed between different DNA molecules. Or, the effector region of a DNA construct of the present invention can be a DNA constituted of a sequence that can be hybridized with the telomeric region of a genome, and in this case, with the activation of the effector region, the effector region inhibits the expansion of a telomere by telomerase by stabilizing a

G-quadruplex structure. The relationship between the stabilization of a G-quadruplex structure and cancer treatment is mentioned in, for example, Balasubramanian S., Hurley L. H., Neidle S., Targeting G-quadruplexes in gene promoters: a novel anticancer strategy? (2011) Nat. Rev. Drug. Discov. 10 (4):261-75. A method for selecting a DNA sequence that is hybridized with other nucleic acid molecules only when the effector region is activated, and a method for adjusting the conditions for the hybridization (salt intensity, concentration of surfactants, temperature, etc.) are well known for persons skilled in the art.

A DNA construct of the present invention can exert activity preferably 2-fold or more, more preferably 3-fold or more, and still more preferably 4-fold or more higher than that in the absence of ligands, by ligand-dependently activating the effector region when a ligand binds to the DNA aptamer region. Activity of the effector region is enzymatic activity of the signal-generating region when the effector region is a signal-generating region, and is the amount of nucleic acid molecules to be hybridized when the effector region is a DNA that is hybridized with other nucleic acid molecules. For example, in a DNA construct of the present invention, the activity of the effector region at a ligand concentration of 5 mM is preferably 2-fold or more, more preferably 3-fold or more, still more preferably 4-fold or more, and yet more preferably 5-fold or more higher than the activity of the effector region at a ligand concentration of 0 mM. With regard to the degree of activation, for example, when the signal-generating region is a redox DNAzyme, using a sensor element or a microarray of the present invention, a redox current generated by the redox DNAzyme may be measured to compare the measurement value between in the presence and absence of ligands. Specifically, for example, using a sensor element or microarray of the present invention in which a DNA construct of the present invention having a redox DNAzyme as an effector region is supported, when a redox current generated by the redox DNAzyme is measured to compare the measurement value between in the presence and absence of ligands, the electrical signal at a ligand concentration of 5 mM is preferably 2-fold or more, more preferably 3-fold or more, still more preferably 4-fold or more, and yet more preferably 5-fold or more higher than the electrical signal at a ligand concentration of 0 mM. The amount of nucleic acid molecules to be hybridized can be measured with well-known methods for persons skilled in the art using enzymatic activity, fluorescence, or RI.

Activity of a DNA construct of the present invention increases preferably at a desired ligand concentration range. In other words, in a DNA construct of the present invention, when a ligand at the highest concentration in a desired ligand concentration range is added, the effector region can exert activity preferably 2-fold or more, more preferably 3-fold or more, still more preferably 4-fold or more, and yet more preferably 5-fold or more higher than that at the lowest concentration. In other words, when a ligand at the highest concentration in a desired ligand concentration range is added, enzymatic activity or the amount of hybridization by the effector region is preferably 2-fold or more, more preferably 3-fold or more, still more preferably 4-fold or more, and yet more preferably 5-fold or more higher than that at the lowest concentration. For example, using a sensor element or microarray of the present invention in which a DNA construct of the present invention having a redox DNAzyme as an effector region is supported, when a redox current generated by the redox DNAzyme is measured to compare the measurement value in the presence of ligands with that in the absence of ligands and a ligand at the highest concentration in the concentration range is added, the electrical signal is preferably 2-fold or more, more preferably 3-fold or more, still more preferably 4-fold or more, and yet more preferably 5-fold or more higher than that at the lowest concentration.

As used herein, a “DNA aptamer region” is a region constituted of a DNA (DNA aptamer) having an ability to bind to ligands and causing a change in its secondary structure by binding to ligands. DNA used as such DNA aptamer region includes patulin aptamer, AMP aptamer, and arginine aptamer, etc. AMP aptamer is preferably an AMP aptamer having the sequence of SEQ ID NO: 17. In the case of AMP aptamer, adenosine or adenosine triphosphate (ATP) can also be a ligand. Arginine aptamer is preferably an arginine aptamer having the sequence of SEQ ID NO: 18.

Patulin aptamer used as a DNA aptamer region can be, for example, a DNA molecule having the base sequence of SEQ ID NO: 24, SEQ ID NO: 25, or SEQ ID NO: 26, or a DNA molecule having a sequence homologous to these base sequences. In this case, a DNA molecule of the present invention can be 25 nucleotides length to 35 nucleotides length, preferably 30 nucleotides length.

For example, a DNA molecule having a sequence homologous to the base sequence of SEQ ID NO: 24, SEQ ID NO: 25, or SEQ ID NO: 26 includes a DNA molecule having a base sequence showing 80% or more, 85% or more, 90% or more, or 95% or more sequence identity to the base sequence of SEQ ID NO: 24, SEQ ID NO: 25, or SEQ ID NO: 26, or a DNA molecule that is hybridized with a DNA molecule having a complementary sequence to the base sequence of SEQ ID NO: 24, SEQ ID NO: 25, or SEQ ID NO: 26. (This DNA molecule is constituted of at least 25 bases, at least 26 bases, at least 27 bases, at least 28 bases, at least 29 bases, or at least 30 bases, and the full length can be 30 bases, 31 bases, 32 bases, or 35 bases.) A DNA molecule having a sequence homologous to the base sequence of SEQ ID NO: 24, SEQ ID NO: 25, or SEQ ID NO: 26 can be a DNA molecule having a base sequence with substitution, insertion, or deletion of 1 base to 5 bases, more preferably 1 base to 4 bases, still more preferably 1 base to 3 bases, and yet more preferably 1 base preferably for a DNA molecule having the base sequence of SEQ ID NO: 24, SEQ ID NO: 25, or SEQ ID NO: 26. A DNA molecule of the present invention can be a DNA molecule having a base sequence with deletion of 1 base to 5 bases, preferably 1 base to 4 bases, more preferably 1 base to 3 bases, and still more preferably 1 base, although not particularly limited, for a DNA molecule having the base sequence of SEQ ID NO: 24, SEQ ID NO: 25, or SEQ ID NO: 26. Deletion of bases can be performed, for example, at the end of a sequence (the 5′ end or the 3′ end).

In a DNA construct of the present invention, at least part of the construct activates an effector region masked in the absence of ligands via a change in the secondary structure of the DNA aptamer region. The DNA aptamer region is constituted of at least one or more serially-connected DNA aptamer, preferably constituted of 2 to 3 DNA aptamers, most preferably constituted of 1 DNA aptamer. When the DNA aptamer region is constituted of plural DNA aptamers, each DNA aptamer may be directly connected each other, or each DNA aptamer may be connected via a linker sequence (although not particularly limited, for example, about 1 to 10 bases length) between each aptamer. A DNA aptamer region constituted of individual DNA aptamer is a DNA aptamer region forming 1 or more loop structure(s) in the absence of ligands, preferably a DNA aptamer region forming 1 to 3 loop structure(s), and more preferably a DNA aptamer region forming 1 loop structure. When described based on a concrete example, although not particularly limited, for example, when a DNA aptamer region is constituted of 1 DNA aptamer and 1 DNA aptamer forms 2 or more loop structures in the absence of ligands (i.e., when it forms a clover-structured DNA aptamer region), a DNA construct of the present invention can be a DNA construct forming 2 or more loop structures in the absence of ligands, and when the DNA aptamer forms only 1 loop structure (i.e., it forms a loop-structured DNA aptamer region), a DNA construct of the present invention is a DNA construct forming a hairpin loop structure in the absence of ligands (i.e., a hairpin-loop-structured DNA construct). As another example, when a DNA aptamer region is constituted of 2 or more DNA aptamers, a DNA construct of the present invention can be a DNA construct forming 2 or more loop structures in the absence of ligands. Therefore, a DNA construct of the present invention is a DNA construct forming at least one loop structure in the absence of ligands.

In a certain preferable aspect, a DNA construct of the present invention is a DNA construct forming 1 loop structure in the absence of ligands, including a DNA aptamer region forming only 1 loop structure, an aptamer mask region, a junction region 1, a junction region 2, an effector region, and a terminal region, and in other words, in a certain preferable aspect, a DNA construct of the present invention is a hairpin-loop-structured DNA construct, including a loop-structured DNA aptamer region, an aptamer mask region, a junction region 1, a junction region 2, an effector region, and a terminal region.

As used herein, an “aptamer mask region” is a region masking part of the DNA aptamer region in the absence of ligands. When a ligand binds to the DNA aptamer region, this masking is removed and the effector region is activated. The length of the aptamer mask region is 3 to 5 bases length, preferably 4 or 5 bases length, and more preferably 4 bases length.

As used herein, a “junction region” is a region connecting an effector region and a DNA aptamer region. As used herein, a region connected with an aptamer mask region is referred to as junction region 1, and a region connected with a DNA aptamer region is referred to as junction region 2. The junction region 1 and the junction region 2 are hybridized each other in the absence of ligands, but when a ligand binds to a DNA construct, one junction region is dissociated from the other. The junction region 1 and the junction region 2 are 1 to 5 bases length, preferably 2 to 5 bases length, more preferably 3 to 5 bases length, and still more preferably 3 bases length. The length of the junction region 1 and the junction region 2 is preferably the same length. Therefore, preferably, the length of the junction region 1 and the junction region 2 is the same and 1 to 5 bases length, preferably 2 to 5 bases length, more preferably 3 to 5 bases length, and still more preferably 3 bases length.

As used herein, a “terminal region” is a region that exists at the 5′ or 3′ end of a DNA construct, and is hybridized with at least part of an effector region to inactivate the effector region in the absence of ligands to the DNA aptamer region. A terminal region of a DNA construct of the present invention can be referred to as an effector mask region since when a ligand binds to the DNA construct, it is dissociated from the effector region to activate the effector region. There is no particular limitation on the terminal region, provided that the terminal region can be hybridized with at least part of the effector region to inactivate the effector region, but the terminal region is preferably 2 to 5 bases length, more preferably 3 bases length or 4 bases length, and still more preferably has a sequence complementary to the sequence that is masked within the effector region.

As used herein, a region that is formed by an aptamer mask region, a junction region 1, and junction region 2 in the absence of ligands, and that connects a DNA aptamer region and an effector region is referred to as “module region”. The module region itself is not required to have enzymatic activity or activity to bind to compounds, but plays a role in transmission of a change in the secondary structure of the DNA aptamer region due to binding of ligands to the effector region.

As used herein, all of the secondary structure of a DNA molecule and the free energy (dG) when a DNA molecule forms a secondary structure are mentioned as the secondary structure and the free energy (dG) expected under prediction conditions of folding temperature of 37° C., Na⁺ concentration of 1 M, and Mg²⁺ concentration of 0 M, using a DNA secondary structure prediction program (UNAfold Version 3.8 that is provided without charge by University at Albany, The State University of New York (http://mfold.rna.albany.edu)). In the UNAfold Version 3.8, for prediction of DNA secondary structure, a secondary structure with minimum free energy when a secondary structure such as stem structure or loop structure is formed within a molecule (optimum structure) is predicted as a secondary structure of the DNA. Therefore, in the UNAfold Version 3.8, together with DNA secondary structure, the free energy (dG) when the secondary structure is formed is expected. Among the secondary structures to be predicted, a structure without a base with which a base pair is formed and a mismatched base pair in a double-stranded nucleic acid are referred to as bulge loop and internal loop, respectively (however, in the UNAfold Version 3.8, a mismatched base pair between T-G is evaluated as forming a base pair and not evaluated as forming an internal loop). Thus, in the present description, a mismatched base pair between T-G will be mentioned as not forming an internal loop, and the number of hydrogen bonds formed by a mismatched base pair between T-G will be counted as 2, but the mismatched base pair will be mentioned in distinction to a normal base pair. An increase in dG (ddG) of a secondary structure of the whole molecule due to the mismatched base pair within a DNA molecule is evaluated by a nearest neighbor method (nearest neighbor free energy) and calculated by considering the type of the base pair adjacent to a base pair or a mismatched base pair in the UNAfold Version 3.8 (SantaLucia, J. Jr., A unified view of polymer, dumbbell, and oligonucleotide DNA nearest-neighbor thermodynamics. Proc. Natl. Acad. Sci. U.S.A. (1998) 95:1460-1465; and SantaLucia, J. Jr., Hicks, D. The thermodynamics of DNA structural motifs. Annu. Rev. Biophys. Biomol. Struct. (2004) 33:415-440). A secondary structure estimated in the present invention is preferably an optimum structure (structure with minimum dG) obtained by prediction of DNA secondary structure, but may include a structure predicted as a suboptimum structure (structure with not minimum but near minimum dG).

As used herein, a “base pair” means a base pair between A-T and a base pair between G-C, and a “mismatched base pair” means a base pair with a combination other than the above combinations. As used herein, to form plural “base pairs” between a certain region and another region of a DNA molecule is referred to as to “hybridize.”

The relationship between the aptamer mask region, the junction region 1, and the junction region 2 in a DNA construct in the present invention will be described below.

As used herein, the relationship between the aptamer mask region, the junction region 1, and the junction region 2 will be described in detail based on an example of a DNA construct (a). For DNA constructs (b), (c), and (d), the terms of “3′” and “5′” in the description below can be appropriately read as “5′” and “3′,” respectively, by checking them against the above connection order.

In a DNA construct (a) of the present invention, in the absence of ligands, 4 to 7 bases at the 3′ end of the aptamer mask region are hybridized with those of the DNA aptamer region, and the junction region 1 is hybridized with the junction region 2. Also in a DNA construct of the present invention, in the absence of ligands, at least part of the effector region is hybridized with the terminal region located at the 3′ end region of the DNA construct to be masked, and as a result, the effector region is inactivated.

In a DNA construct (a) of the present invention, in the absence of ligands, the hybridization of 4 to 7 bases at the 3′ end of the DNA aptamer region with the aptamer mask region and/or the hybridization of the junction sequence 1 with the junction sequence 2 preferably do not form complete hybridization (in a hybridized region, at least one base forms no base pair), and more preferably form at least one internal loop or bulge loop. The internal loop, although not particularly limited, is preferably an internal loop formed by 2 or 3 bases. Therefore, in the present invention, a DNA construct in which the aptamer mask region forms at least one bulge loop or internal loop between bases in this region and the DNA aptamer region to be hybridized is provided. The present invention also provides a DNA construct in which the junction region 1 forms at least one bulge loop or internal loop between bases in this region and the junction region 2. The present invention further provides a DNA construct in which the aptamer mask region forms at least one bulge loop or internal loop between bases in this region and the DNA aptamer region to be hybridized, and the junction region 1 forms at least one bulge loop or internal loop between bases in this region and the junction region 2.

In the present invention, a DNA construct (a) of the present invention, in the absence of ligands, forms preferably a total of 4 to 11 hydrogen bonds, more preferably a total of 4 to 10 hydrogen bonds, and most preferably 6 to 9 hydrogen bonds between 4 to 7 bases at the 3′ end of the DNA aptamer region and bases in the aptamer mask region. In other words, the aptamer mask region of a DNA construct (a) of the present invention, in the absence of ligands, forms 2 base pairs and 1 T-G mismatched base pair or 3 or 4 base pairs between this region and the terminal part of the 3′ side of the DNA aptamer.

Therefore, a DNA construct (a) of the present invention, in the absence of ligands, forms preferably at least one internal loop or bulge loop between 4 to 7 bases at the 3′ end of the DNA aptamer region and the aptamer mask region, and forms preferably a total of 4 to 11 hydrogen bonds, more preferably a total of 4 to 10 hydrogen bonds, and most preferably 6 to 9 hydrogen bonds between these bases. More specifically, a DNA construct (a) of the present invention, preferably, in the absence of ligands, forms 4 base pairs and 1 internal loop or bulge loop, 3 base pairs, 1 T-G mismatched base pair, and 1 internal loop or bulge loop, 3 base pairs and 1 internal loop, or 2 base pairs, 1 T-G mismatched base pair, and 1 internal loop between 4 to 7 bases at the 3′ end of the DNA aptamer region and the aptamer mask region. The above 2 base pairs, 1 T-G mismatched base pair, and 1 internal loop formed between 4 bases at the 3′ end of the DNA aptamer region and the aptamer mask region in the absence of ligands are constituted of preferably 2 A-T base pairs, 1 T-G mismatched base pairs, and 1 internal loop, and more preferably a connection of 2 A-T base pairs, 1 internal loop, and 1 T-G mismatched base pair from the near side of the DNA aptamer region.

In one aspect of the present invention, in a DNA construct (a) of the present invention, a DNA aptamer region forming a hydrogen bond between bases of this region and the aptamer mask region in the absence of ligands is preferably 4 bases at the 3′ end of the DNA aptamer region.

In a certain aspect, in a DNA construct (a) of the present invention, the aptamer mask region is T-(X)ₙ-T-T from the 5′ side, and 4 bases at the 3′ end of the DNA aptamer region are A-A-Z-G from the 5′ side, provided that, n is 1 or 2; when n is 2, two (2) Xs may be the same base or different bases, and X and Z are selected from a combination of bases in which (X)ₙ and Z form an internal loop or a bulge loop; and when n is 1, X and Z are selected from a combination of bases forming an internal loop between X and Z. For reference, for example, in a DNA (b) of the present invention, when the DNA aptamer region is a DNA construct adjacent to the 5′ side of the aptamer mask region, the aptamer mask region is T-T-(X)ₙ-T from the 5′ side, and 4 bases at the 3′ end of the DNA aptamer region are G-Z-A-A from the 5′ side. In this aspect, the DNA aptamer region is preferably an AMP aptamer region, and more preferably an AMP aptamer having the base sequence of SEQ ID NO: 17.

In a certain aspect, in a DNA construct (a) of the present invention, the aptamer mask region is T-C-G-T from the 5′ side, and 4 bases at the 3′ end of the DNA aptamer region are A-A-G-G from the 5′ side. In this aspect, the DNA aptamer region is preferably a patulin aptamer region, more preferably a patulin aptamer region having the base sequence of SEQ ID NOS: 24 to 26, and still more preferably a patulin aptamer region having the base sequence of SEQ ID NO: 26.

In a DNA construct (a) of the present invention, the length of the junction region is 1 to 5 bases length, preferably 2 to 5 bases length, more preferably 3 to 5 bases length, and still more preferably 3 bases length. In the absence of ligands, 3 or more hydrogen bonds are formed between bases in the junction region 1 and the junction region 2 of a DNA construct of the present invention. Between bases in the junction region 1 and the junction region 2, although not particularly limited, the number of G-C base pairs is preferably 0 or 1, preferably 0, and more preferably, all base pairs are constituted of either of an A-T base pair or a T-G mismatched base pair. Between bases in the junction region 1 and the junction region 2, preferably 1 internal loop may exist. In a certain aspect, with regard to the junction region of a DNA construct (a) of the present invention, the junction region 1 is A-G-C (or T-A-G) from the 5′ side, and the junction region 2 is G-A-T from the 5′ side. (When the junction region 1 is T-A-G from the 5′ side, C-G-A from the 5′ side). In this aspect, the DNA aptamer region is preferably an AMP aptamer region, and more preferably, an AMP aptamer having the base sequence of SEQ ID NO: 17.

In a certain aspect, the junction region 1 of a DNA construct (a) is C-T-G (or G-A-T) from the 5′ side, and the junction region 2 is T-A-G (or G-T-C) from the 5′ side. In this aspect, the DNA aptamer region is preferably a patulin aptamer region, more preferably a patulin aptamer region having the base sequence of SEQ ID NOS: 24 to 26, and still more preferably a patulin aptamer region having the base sequence of SEQ ID NO: 26.

In a certain aspect, the base sequence in the terminal region of a DNA construct (a) is C-C-C-A or C-C-C from the 5′ side. According this aspect, the base sequence in the effector region is preferably T-G-G-G or G-G-G from the 5′ side at its 5′ end, and more preferably a base sequence of a redox DNAzyme having the base sequence of SEQ ID NO: 16. When the base sequence in the terminal region of a DNA construct (a) is C-C-C-A from the 5′ side, the aptamer mask region is preferably T-A-T-T from the 5′ side, and 4 bases at the 3′ end of the DNA aptamer region are A-A-G-G from the 5′ side. In this aspect, although not particularly limited, the DNA aptamer region is preferably an AMP aptamer region or a patulin aptamer region.

If a DNA construct of the present invention is over stabilized in the absence of ligands, it cannot cause a structural change at the time of binding of ligands, and as a result, the effector region may be always inactivated in the presence or absence of ligands. On the other hand, if a DNA construct of the present invention is over unstable, hybridization of the effector region with the terminal region is unlikely to be formed even at the time of non-binding of ligands, and as a result, the effector region may be always activated in the presence or absence of ligands. Thus, in order to make a DNA construct of the present invention be at an inactivated state in the absence of ligands and be activated dependent on ligand-binding, it is preferable to make the free energy (dG) when a DNA construct of the present invention forms a secondary structure be within a certain range. Specifically, in a DNA construct of the present invention (in the absence of ligands), the free energy (dG) (kcal/mol) when a DNA construct of the present invention forms a secondary structure can be predicted under a prediction condition 1 with the UNAfold Version 3.8, and the lower limit of the free energy (dG) is preferably −14 kcal/mol, more preferably −12 kcal/mol, still more preferably −10 kcal/mol, and most preferably −9 kcal/mol, and the upper limit of the free energy (dG) is preferably −5 kcal/mol, more preferably −6 kcal/mol, and most preferably −6.5 kcal/mol. Therefore, dG of a DNA construct of the present invention, although not particularly limited, for example, can be −12 to −5 kcal/mol, preferably −10 to −5 kcal/mol, more preferably −9 to −5 kcal/mol, still more preferably −9 to −6 kcal/mol, and most preferably −9 to −6.5 kcal/mol. In this way, when a DNA construct of the present invention is designed, the construct can be designed by considering that expected dG is within the above range.

A DNA construct (a) of the present invention causes conversion of the secondary structure dependent on the binding to ligands to the DNA aptamer region. At this time, in a DNA construct (a) of the present invention, preferably the aptamer mask region is hybridized with the junction region 2 after binding of ligands. After binding of ligands, a DNA construct in which the aptamer mask region is hybridized with the junction region 2 is considered to maintain the free state of the effector region (i.e., activation state), and this is advantageous in achievement of high sensitivity of the DNA construct, compared with when the aptamer mask region is not hybridized with the junction region 2. The aptamer mask region and the junction region 2, although not particularly limited, preferably form 4 or more hydrogen bonds in the hybridization region, more preferably form a base pair with 2 consecutive bases, and most preferably form 3 consecutive base pairs.

In a DNA construct (a) of the present invention, when the aptamer mask region is 4 bases length and the aptamer mask region has a mismatched base pair in the absence of ligands, bases forming a mismatched base pair are selected from a combination of bases so that an increase in dG (ddG) of a secondary structure of the whole molecule due to the mismatched base pair in the aptamer mask region is, although not particularly limited, preferably +0.1 kcal/mol or more, more preferably +0.5 kcal/mol or more, still more preferably +1.0 kcal/mol or more, and most preferably +2.0 kcal/mol or more. In a DNA construct (a) of the present invention, when the aptamer mask region is 4 bases length and the junction region has a mismatch in the absence of ligands, bases forming a mismatched base pair are selected from a combination of bases so that an increase in dG of a secondary structure of the whole molecule due to the mismatch in the junction region is, although not particularly limited, preferably +1.0 kcal/mol or less, more preferably +0.5 kcal/mol or less, still more preferably +0.3 kcal/mol or less, and most preferably +0.1 kcal/mol or less. In a DNA construct of the present invention, when the aptamer mask region is 4 bases length and each of the aptamer mask region and the junction region has a mismatch, bases forming a mismatched base pair are selected from a combination of bases so that an increase in dG of a secondary structure of the whole molecule due to the mismatch in the aptamer mask region is, although not particularly limited, preferably +0.1 kcal/mol or more, more preferably +0.5 kcal/mol or more, still more preferably +1.0 kcal/mol or more, and most preferably +2.0 kcal/mol or more, and/or are selected from a combination of bases so that an increase in dG of a secondary structure of the whole molecule due to the mismatch in the junction region is preferably +1.0 kcal/mol or less, more preferably +0.5 kcal/mol or less, still more preferably +0.3 kcal/mol or less, and most preferably +0.1 kcal/mol or less. Therefore, when a DNA construct of the present invention is designed, the construct can be designed by considering that expected ddG is within the above range. The relationship between a combination of mismatched base pairs and ddG is mentioned in SantaLucia, J. Jr., Hicks, D. The thermodynamic of DNA structural motifs. Annu. Rev. Biophys. Biomol. Struct. (2004) 33:415-440, and this mention can be used as a guideline for selecting bases (or combination of bases) meeting desired ddG.

In a certain aspect, the base sequence of a DNA construct of the present invention is a base sequence in which any 1, 2, 3, or all of the base sequence of 4 bases at the 3′ end of the aptamer mask region, the junction region 1 and the junction region 2, and the DNA aptamer region are selected from any one of the combinations shown in Table 1 below. In a certain aspect, in the base sequence of a DNA construct of the present invention, all base sequences of 4 bases at the 3′ end of the aptamer mask region, the junction region 1 and the junction region 2, and the DNA aptamer region are selected from any one of the combinations shown in Table 1 below.

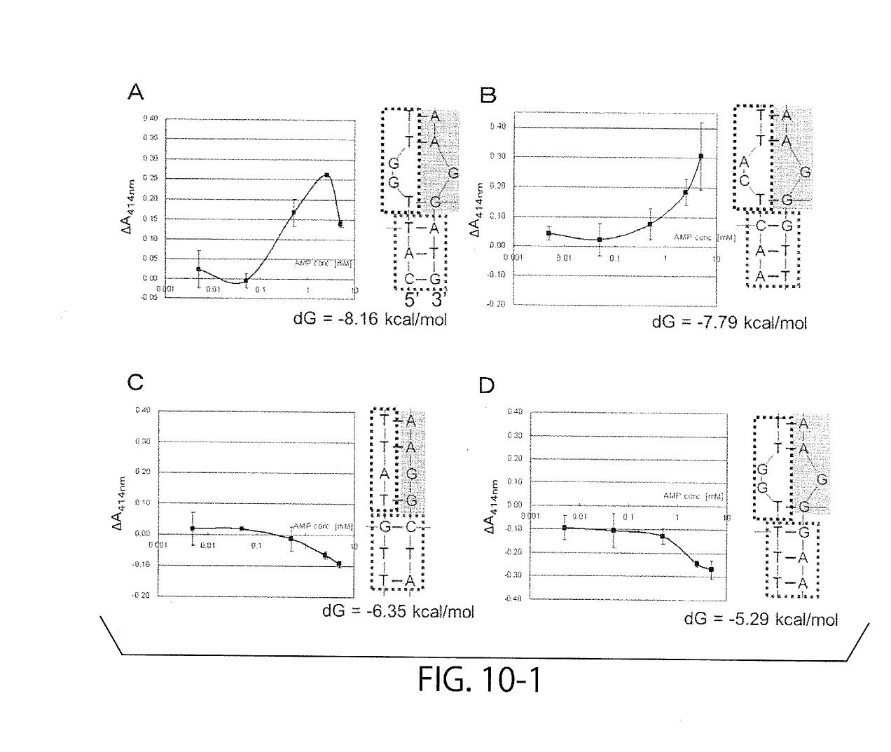

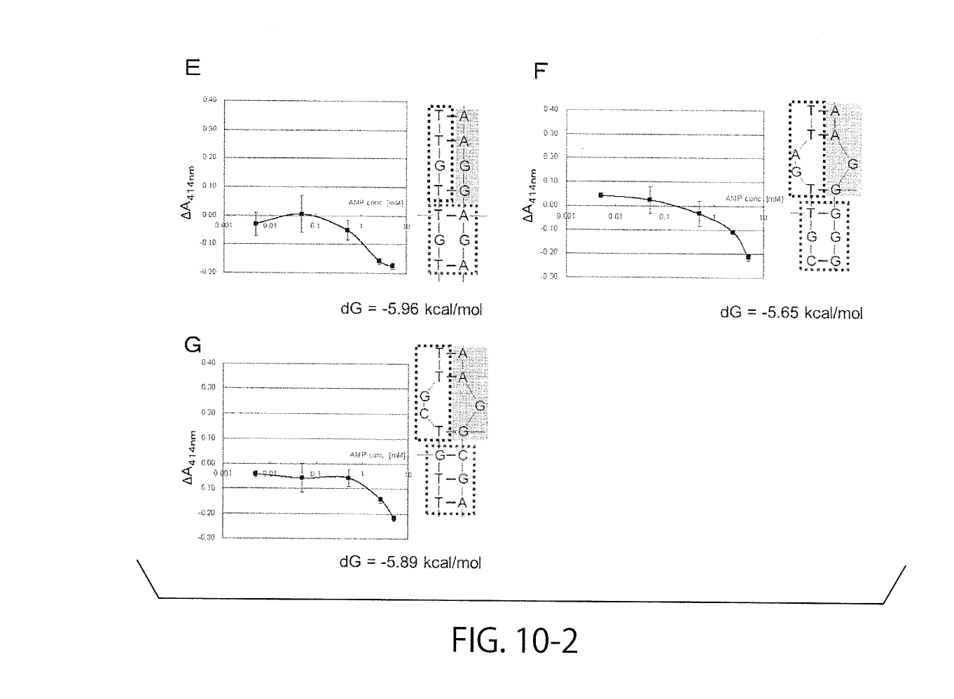

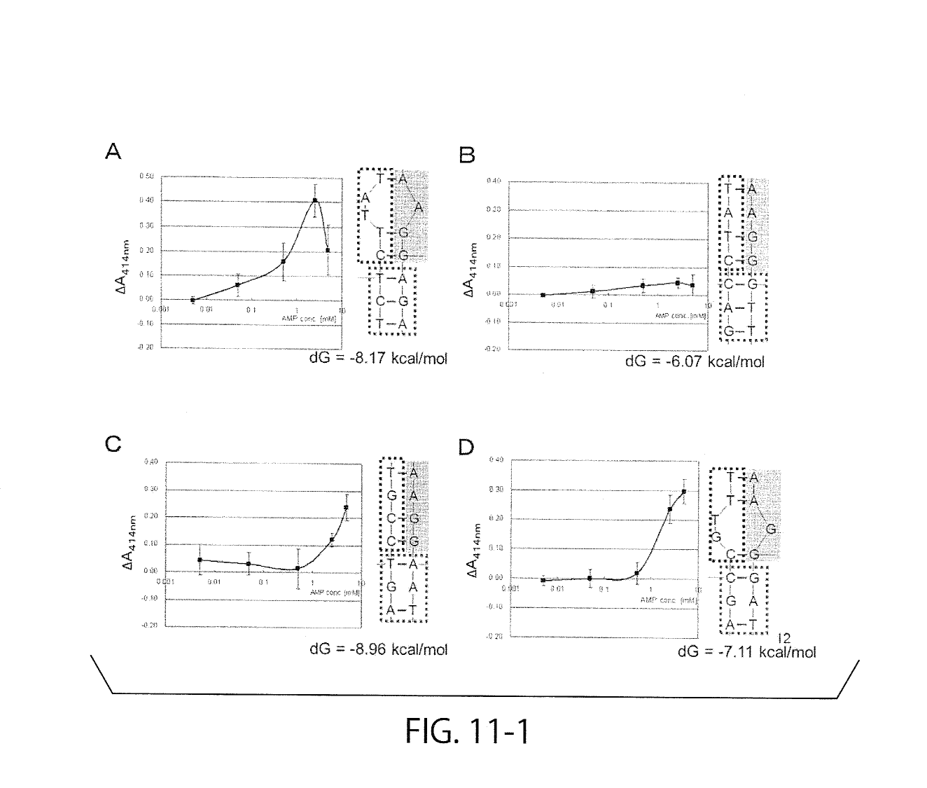

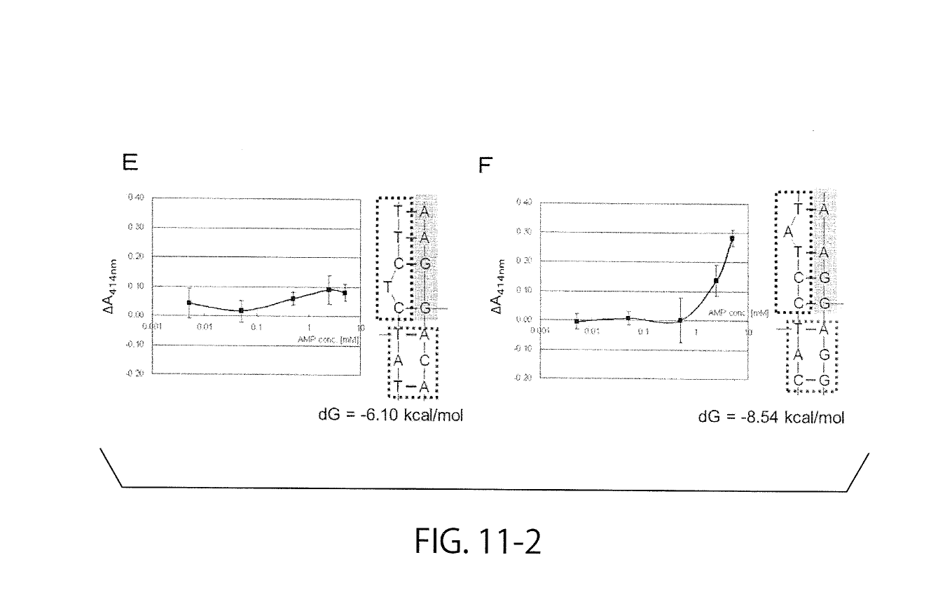

TABLE 1
Table 1: Combination of base sequence in each
region of module regions

| | | | 4 bases | | |
|---|---|---|---|---|---|
| | | | at the | | |
| | | | 3′ end | | |
| | | | of the | | |
| Combi- | Correspond- | Aptamer | DNA | Junction | Junction |
| nation | ing DNA | mask | aptamer | region | region |
| number | molecule | region | region | 1 | 2 |
| 1 | TMP-1 | TATT | AAGG | AAA | TTT |
| 2 | TMP-5 | TATT | AAGG | AGC | GAT |
| 3 | TMP-6 | TGTT | AAGG | ATA | TAT |
| 4 | FIG. 10A | TGGTT | AAGG | CAT | ATG |
| 5 | FIG. 10B | TCATT | AAGG | AAC | GTT |
| 6 | FIG. 11A | CTTAT | AAGG | TCT | AGA |
| 7 | FIG. 11B | CTAT | AAGG | GAC | GTT |
| 8 | FIG. 11C | CCGT | AAGG | AGT | AAT |
| 9 | FIG. 11D | CGTTT | AAGG | AGC | GAT |
| 10 | FIG. 11E | CTCTT | AAGG | TAT | ACA |
| 11 | FIG. 11F | CCTAT | AAGG | CAT | AGG |
| 12 | TMP5-1 | TTAT | AGAG | AGC | GAT |
| 13 | TMP-5-2 | TCAT | AGGG | AGC | GAT |
| 14 | TMP-5-5 | TATC | GAGG | AGC | GAT |
| 15 | TMP5-TG1 | TATT | AGGA | AGC | GAT |
| 16 | SC-7-CCCA- | TCGT | AAGG | CTG | TAG |
| | TMP-7 | | | | |

*Base sequences are mentioned so that the base at the left end is the 5′ side and the base at the right end is the 3′ side.

In a certain aspect, the base sequence of a DNA construct in which a DNA aptamer region of the present invention is an AMP aptamer region is any one of base sequences of SEQ ID NOS: 1 to 15. In a certain aspect, the base sequence of a DNA construct in which a DNA aptamer region of the present invention is a patulin aptamer region is any one of base sequences of SEQ ID NOS: 21 to 23, 40, and 41. In a certain aspect, the base sequence of a DNA construct in which a DNA aptamer region of the present invention is an arginine aptamer region is the base sequence of SEQ ID NO: 19.

A DNA construct of the present invention, for example, can be obtained by modifying the DNA aptamer region of a DNA construct having any one of base sequences of SEQ ID NOS: 1 to 15, 19, 21 to 23, 40, and 41, and by using the binding to each ligand as an index. Modification (substitution, insertion, and deletion of bases) can be performed so that the condition of a DNA construct of the present invention is met. An RNA construct of the present invention in which the RNA aptamer region is a patulin aptamer region, for example, can be obtained by modifying the patulin aptamer region of an RNA construct having the base sequence of SEQ ID NOS: 30 to 32, 38, and 39 and by using patulin-binding-dependent self-cleaving activity as an index. Modification (substitution, insertion, and deletion of bases) can be performed so that the condition of an RNA construct of the present invention is met. A method for modifying a nucleic acid molecule is well known for persons skilled in the art.

The fact that a DNA construct meets the condition of a DNA construct of the present invention is an index for designing a DNA construct showing high sensitivity or specificity to ligands. A DNA construct of the present invention can be designed so that the condition of a DNA construct of the present invention is met. Therefore, according to the present invention, a method for designing a DNA construct for detection of ligands is provided. In a DNA construct of the present invention, by immobilizing the DNA construct obtained by being designed on a microarray of the present as a sensor element, the binding of ligands may be used as an index.

In the present invention, the activation of the effector region of a DNA construct of the present invention can be electrochemically detected using a sensor element having the electrode surface on which a DNA construct of the present invention is supported. Use of a microarray equipped with a sensor element having the electrode surface on which a DNA construct of the present invention is supported can obtain DNA constructs highly sensitive to ligands at a stroke by screening enormous types of DNA constructs, as described below. Therefore, in the present invention, a sensor element in which a DNA construct of the present invention is immobilized on the electrode surface, and a microarray equipped with a sensor element of the present invention are provided. In a DNA construct of the present invention that is immobilized on the electrode surface, the effector region is preferably a signal-generating region, more preferably a DNAzyme, still more preferably a redox DNAzyme, and most preferably a redox DNAzyme of SEQ ID NO: 16.

When a DNA construct of the present invention is immobilized on the electrode surface, a linker can intervene between the DNA construct and the electrode. Therefore, according to the present invention, a sensor element in which a DNA construct is immobilized on the electrode surface via a linker, and a microarray equipped with the sensor element are provided. In the present invention, the linker, although not particularly limited, can be DNA, and its sequence can be preferably a sequence that is not hybridized with other regions within the DNA construct. The sequence that is not hybridized with other regions within the DNA construct can be easily selected by persons skilled in the art. The linker, for example, can be poly T, and the length, for example, can be 1 to 20 bases length. Therefore, in the present invention, the sequence of the linker can be preferably poly T of 1 to 20 bases length, and more preferably poly T of 15 bases length. According to the present invention, a linker intervening between the DNA construct and the electrode can reduce the effects such as steric hindrance of the electrode on the DNA construct, and can improve the detection sensitivity of a ligand. In the present invention, the linker can be added to the 5′ end or the 3′ end of the DNA construct, and preferably can be added to the terminal region (i.e., the end opposed to the end in which the effector region exists) of the DNA construct.

In the present invention, a DNA construct can be immobilized on the electrode surface on a sensor element by various publicly known methods, and for example, preferably by spotting a DNA construct on the electrode on a sensor element, and more preferably by synthesizing a DNA construct on the electrode on a sensor element. When a DNA construct is synthesized on the electrode surface of a sensor element, synthesized DNA can be immobilized on the electrode surface with constant orientation, and thus this method is preferable in terms of the detection sensitivity of a ligand. Therefore, in the present invention, by synthesizing a DNA construct on a sensor element, a sensor element in which the DNA construct is immobilized on the electrode surface is provided. In the present invention, use of a microarray equipped with a sensor element of the present invention can synthesize a plurality, preferably 1,000 types or more, more preferably 5,000 types or more, and still more preferably 10,000 types or more of DNA constructs on the electrode surface of 1 microarray, and can screen these enormous types of DNA constructs at a stroke. A DNA construct can be synthesized on the electrode surface of a sensor element by various publicly known methods as methods for array manufacturing, and for example, by the method disclosed in the Japanese Unexamined Patent Publication No. 2006-291359, etc. In the present invention, there is no particular limitation on the microarray, and for example, an ElectraSense (trademark) microarray manufactured by CustomArray Inc. can be used.

In the present invention, using a sensor element of the present invention or a microarray equipped with the sensor element, ligands can be detected by measuring electrical signal in the presence of a substrate of a DNAzyme. A detection method for ligands of the present invention can detect ligands by reading a current generated by a DNAzyme on the electrode of the sensor element. The DNAzyme, although not particularly limited, is preferably a redox DNAzyme, and more preferably a redox DNAzyme of SEQ ID NO: 16. In the present invention, ligands can be detected by reading a redox current generated by activation of a redox DNAzyme on the electrode of the sensor element.

In a detection method for ligands of the present invention, a sequence that can be hybridized with other nucleic acid molecules only in the presence of ligands, i.e., a DNA construct having a sequence that can be hybridized with a nucleic acid molecule conjugating oxidoreductase such as HRP only in the presence of ligands can also be used as an effector region. When such DNA construct is used, ligands can be detected by immobilizing a DNA construct on a sensor element, by contacting it with a nucleic acid molecule conjugating oxidoreductase such as HRP under a condition in which hybridization is possible, and then by detecting a redox current generated by oxidoreductase.

In the present invention, a method for screening for a DNA construct with the detection sensitivity of a ligand as an index using methods of the present invention from the DNA construct candidate group obtained by further modifying the base sequence of a DNA construct of the present invention is also provided.

A screening method of the present invention is a screening method for a DNA molecule for detection of ligands or for a nucleic acid molecule having a base sequence equivalent thereto, including the following processes:

(A) obtaining a DNA molecule candidate group for detection of ligands or a nucleic acid molecule having a base sequence equivalent thereto by designing or modifying the base sequence of a DNA molecule, which is composed of a DNA aptamer region, a module region, and an effector region that is activated dependent on the binding of ligands to the DNA aptamer region, and also forms a loop structure in the absence of ligands,

(B) fabricating a microarray equipped with a sensor element in which a DNA molecule or a nucleic acid molecule having the obtained base sequence is immobilized on the electrode surface,

(C) electrochemically measuring the redox current from the effector region using the obtained microarray, and (D) selecting a DNA molecule or a nucleic acid molecule using the detection sensitivity of a ligand as an index.

The above processes (A) to (D) will be described below by process.

### Re: Process (A)

In the present invention, the screening begins with obtainment of a base sequence group of DNA molecules that includes a DNA aptamer region, a module region, and an effector region, and that forms a loop structure in the absence of ligands. In a screening method of the present invention, there is no particular limitation on design of the base sequence of a DNA molecule, and for example, the design can be performed using a DNA construct of the present invention as an index. Or, when a DNA construct of the present invention is not used as an index, design of the base sequence of a DNA molecule may be performed using the feature that the DNA molecule shows minimum dG of a secondary structure in which the effector region is masked in the absence of ligands and that dG of a secondary structure in which the masking of the effector region is removed dependent on ligand-binding shows minimum as an index. In a screening method of the present invention, a DNA molecule to be modified is a DNA molecule in which when a ligand binds to the DNA aptamer region, the effector region is activated and the effector region becomes able to be hybridized with other nucleic acid molecules conjugating oxidoreductase, or when it is a signal-generating region, a DNA molecule that activates its oxidoreductase activity. In a screening method of the present invention, as a DNA molecule to be modified, for example, a DNA molecule in which a DNA aptamer and a DNAzyme are connected to form, for example, a hairpin loop structure can be used, and although not particularly limited, for example, a DNA construct of the present invention can be used, and a DNA molecule having preferably any one of base sequences of SEQ ID NOS: 1 to 15 when a ligand is AMP, preferably the base sequence of SEQ ID NO: 19 when a ligand is arginine, and preferably any one of base sequences of SEQ ID NOS: 21 to 23, 40, and 41 when a ligand is patulin. In the process (A′), like the procedure in the process (A), a DNA molecule candidate group for detection of ligands by further modifying the DNA molecules already obtained by a screening method of the present invention, or a nucleic acid molecule having a base sequence equivalent thereto may be obtained. In the present invention, the effector region of a DNA molecule to be screened can be preferably a signal-generating region, more preferably a DNAzyme, still more preferably a redox DNAzyme, and yet more preferably a redox DNAzyme having the sequence of SEQ ID NO: 16. As a nucleic acid having a base sequence equivalent to the base sequence of the obtained DNA molecule, by substituting part of the obtained DNA molecule (for example, at least one part selected from the group constituted of an effector region, a junction region 1, an aptamer mask region, a DNA aptamer region, a junction region 2, and a terminal region by a nucleic acid other than DNA (e.g., PNA, LNA, or RNA), a nucleic acid molecule can be obtained. In a certain aspect of the present invention, the process (A) is to obtain a DNA molecule candidate group for detection of ligands or a nucleic acid molecule having a base sequence equivalent thereto by designing or modifying the base sequence of a hairpin-loop-structured DNA molecule, which is composed of a loop-structured DNA aptamer region, a module region, and an effector region that is activated dependent on the binding of ligands to the DNA aptamer region.

Modification of the base sequence of a DNA molecule can be performed for 1 or more regions selected from a DNA aptamer region, a module region, an effector region, and other region(s) (e.g., a terminal region), and preferably can be performed by selecting any one region. Modification of the base sequence of a DNA molecule can be performed by 1 or more procedure selected from insertion, deletion, and substitution of bases. Modification of the base sequence of a DNA molecule can be performed by substituting the effector region by one in which activity can easily be measured, for example, a redox DNAzyme, in terms of making screening simple. Thus, even a DNA molecule having an effector region that is conventionally difficult to be screened can be screened by a screening method of the present invention. Regardless of particular methods, modification can be appropriately performed with well-known methods for persons skilled in the art, and preferably can be performed on computer.

For example, in the present invention, preferably using a DNA molecule secondary structure prediction program such as UNAfold Version 3.8, only a molecule in which the optimum structure (or suboptimum structure) is estimated to form a desired secondary structure (e.g., hairpin loop structure) in the absence of ligands can be subjected to screening. For example, when a DNA construct (a) of the present invention is used as a DNA molecule, for example, only a DNA molecule that is estimated to form a hairpin loop structure like FIG. 1A or FIG. 2A in the absence of ligands can be subjected to screening. When a base sequence is modified on computer, although not particularly limited, modification can be performed, for example, using meeting the condition of a DNA construct of the present invention, e.g., the fact that dG of a DNA molecule having a modified base sequence meets the condition of a DNA construct of the present invention, as an index. In this way, in the present invention, a molecule that can show high sensitivity can be selectively subjected to screening, and the present invention has higher screening efficiency than that of a method for randomly introducing mutations to screen all of them.

### Re: Process (B)

In the present invention, as mentioned above, a sensor element in which a DNA molecule is immobilized on the electrode surface can be fabricated. In a screening method of the present invention, preferably a microarray equipped with a sensor element of the present invention is used.

### Re: Process (C)

In the present invention, a redox current can be measured in accordance with the manufacture's manual, using an electrochemical detector for microarray and a microarray for electrochemical detection. There is no particular limitation on an electrochemical detector for microarray, and for example, an ElectraSense (trademark) detector manufactured by CustomArray Inc. can be used, and there is no particular limitation on a microarray for electrochemical detection, and for example, an ElectraSense (trademark) microarray manufactured by CustomArray Inc. can be used.

### Re: Process (D)

In the present invention, the magnitude of the measured current value reflects the detection sensitivity of a ligand of a DNA molecule or a nucleic acid molecule. For example, greater difference (or ratio) in the current value measured between in the absence and presence of ligands means that a DNA molecule or a nucleic acid molecule has higher detection sensitivity of a ligand. Therefore, in the present invention, for example, a DNA molecule or a nucleic acid molecule having high ligand detection sensitivity can be selected based on the measured current value. When a DNA molecule or a nucleic acid molecule is screened, for example, a molecule having higher ligand detection sensitivity than the mean sensitivity of the whole molecule may be selected, or some of DNA molecules or nucleic acid molecules having the highest ligand detection sensitivity may be selected.

In the candidate sequence group obtained in the process (A), a ligand concentration range in which high quantitativity can be exerted by DNA molecule or nucleic acid molecule is considered to be different. In other words, a certain DNA molecule or nucleic acid molecule exerts high quantitativity at a low concentration range while other DNA molecules exert high quantitativity at a high concentration range, which shows that the best concentration range differs by molecule. Thus, in order to obtain a DNA molecule or nucleic acid molecule that exerts high quantitativity at a desired concentration range, screening can be performed based on the detection sensitivity of a ligand at the desired concentration range. Therefore, in the present invention, a DNA molecule or a nucleic acid molecule can be screened using the detection sensitivity of a ligand at a desired concentration range as an index. For example, when a DNA molecule or a nucleic acid molecule that exerts high sensitivity at a concentration range between 0 mM to 5 mM is obtained, a DNA molecule or a nucleic acid molecule can be screened using the detection sensitivity of a ligand at the concentration range as an index. When a DNA molecule or a nucleic acid molecule that exerts high detection sensitivity at a concentration range between 5 mM to 10 mM is obtained, a DNA molecule or a nucleic acid molecule can be screened using the detection sensitivity of a ligand at the concentration range as an index.

In a screening method of the present invention, after the process (D), the effector region may be further substituted by other effector regions. For example, even in a DNA molecule or a nucleic acid molecule having an effector region without redox activity, for example, by substituting the effector region by a redox DNAzyme, by selecting a highly sensitive DNA molecule or nucleic acid molecule using the detection sensitivity of a ligand as an index, and by substituting the redox DNAzyme portion of the obtained DNA molecule or nucleic acid molecule by the original effector without redox activity, a DNA molecule or a nucleic acid molecule for highly sensitive detection of ligands can be obtained. Similarly, in a screening method of the present invention, after the process (D), the DNA aptamer region may be further substituted by other DNA aptamer regions. By performing such procedure, a screening of the present invention can be applied even to a DNA molecule or a nucleic acid molecule that is difficult to be screened.

In the present invention, the detection sensitivity of a ligand by a DNA molecule or a nucleic acid molecule can be optimized by further modifying the sequence of a DNA molecule or a nucleic acid molecule screened using the detection sensitivity of a ligand as an index and by making the molecule be subjected to further screening. Therefore, in the present invention, an optimization method for the base sequence of a DNA molecule or a nucleic acid molecule forming a loop structure including a DNA aptamer region and an effector region is provided. For this purpose, modification of a DNA molecule or a nucleic acid molecule may be performed for at least one region selected from a DNA aptamer region, a module region, an effector region, and other regions (e.g., terminal region) to optimize the whole DNA molecule and the whole nucleic acid molecule, or by selecting any one region to design or modify the molecule, the one region may be intensively optimized. After one region is intensively optimized, other regions may be optimized. In this way, by repeating the screening, a DNA molecule or a nucleic acid molecule having high sensitivity for a certain ligand can be manufactured and obtained.

In the present invention, after the whole or part of a DNA molecule or a nucleic acid molecule is optimized, part of the DNA molecule or the nucleic acid molecule (e.g., one region) may be substituted. In other words, after the whole or part of a DNA molecule or a nucleic acid molecule is optimized, the effector region may be substituted by other desired effector regions or DNA that is expected to have a function as an effector region. By this method, optimization of at least portions other than effector region is possible even in a DNA molecule or a nucleic acid molecule having an effector region that is difficult to be screened or optimized. Like effector region, after the whole or part of a DNA molecule or a nucleic acid molecule is optimized, the DNA aptamer region may be substituted by other DNA aptamer regions.

A screening method of the present invention may further include:

(E) selecting a DNA molecule or a nucleic acid molecule showing no binding or weaker binding than that to patulin to compounds other than ligands, for example, compounds other than patulin when a ligand is patulin, for further example, patulin analogs such as theophylline, benzofuran, and (S)-patulin methylether. Evaluation of binding can be performed by a colorimetric test using ABTS or an electrochemical method as mentioned above. Compounds other than ligands, for example, compounds other than patulin, or ligand analogs, for example, patulin analogs can be freely set by persons skilled in the art depending on what type of binding specificity is in the DNA molecule or the nucleic acid molecule. Specifically, in order to obtain a DNA molecule or a nucleic acid molecule showing no binding to a certain compound, the DNA molecule or the nucleic acid molecule can be selected depending on binding to the compound.

In one aspect of the present invention, optimization of the detection sensitivity of a ligand of the present invention does not include a process of artificial molecular evolution such as the SELEX (systematic evolution of ligands by exponential enrichment) method, specifically, a process of introduction of mutation using error prone DNA polymerase, etc. In the present invention, since a DNA molecule is correctly synthesized in accordance with the sequence designed on the electrode of a microarray, the sequence of a DNA molecule that showed high sensitivity has already been grasped as the designed sequence. Therefore, without introducing mutations using enzymes, etc., the base sequence of a DNA molecule can be optimized by further modifying the base sequence based on the sequence information on a DNA molecule on each spot that showed high detection sensitivity in a microarray.

In the present invention, by comparing DNA molecules or nucleic acid molecules obtained by screening or DNA molecules or nucleic acid molecules obtained by optimization, a condition that a DNA molecule or a nucleic acid molecule should meet for improvement of the detection sensitivity of a ligand or a guideline for designing a base sequence of DNA can be obtained.

In the present invention, by substituting part of the DNA molecule obtained by screening by an equivalent nucleic acid other than DNA (e.g., RNA, PNA, or LNA), a nucleic acid molecule candidate group for detection of ligands may be obtained, and in this case, by fabricating a microarray equipped with a sensor element in which each nucleic acid molecule of the candidate group is immobilized on the electrode surface, by electrochemically measuring a redox current from the effector region (especially, constituted of a redox DNAzyme) using the obtained microarray, and by selecting a nucleic acid molecule using the detection sensitivity of a ligand as an index, a nucleic acid molecule for highly sensitive detection of ligands may be screened.

Further, a nucleic acid molecule provided by the present invention will be described.

A nucleic acid molecule provided by the present invention is a patulin-binding nucleic acid molecule. A nucleic acid molecule of the present invention is considered to form a higher-order structure in aqueous solution by an intramolecular hydrogen bond and bind to patulin. Therefore, a nucleic acid molecule of the present invention is a nucleic acid molecule that can form a higher-order structure in aqueous solution.

A nucleic acid molecule of the present invention specifically binds to patulin. In other words, a nucleic acid molecule of the present invention shows binding to patulin, but shows no binding or weaker binding than that to patulin to other compounds with a similar structure (e.g., benzofuran, (S)-patulin methylether, and theophylline, etc.). A nucleic acid molecule of the present invention showing binding specificity for patulin can be advantageously used for detection of patulin in a measurement sample or removal of a patulin molecule from a sample. A DNA molecule of the present invention, an RNA molecule of the present invention, and a nucleic acid construct of the present invention can also be advantageously used for detection of patulin in a measurement sample or removal of a patulin molecule from a sample.

An RNA molecule of the present invention can be, for example, an RNA molecule having the base sequence of SEQ ID NO: 33, SEQ ID NO: 34, or SEQ ID NO: 35, or an RNA molecule having a sequence homologous to these base sequences. In this case, an RNA molecule of the present invention can be 30 nucleotides length to 40 nucleotides length, preferably 30 nucleotides length to 35 nucleotides length, and more preferably 35 nucleotides length.

For example, an RNA molecule having a sequence homologous to the base sequence of SEQ ID NO: 33, SEQ ID NO: 34, or SEQ ID NO: 35 includes an RNA molecule having a base sequence showing 80% or more, 85% or more, 90% or more, or 95% or more sequence identity to SEQ ID NO: 33, SEQ ID NO: 34, or SEQ ID NO: 35, or an RNA molecule that is hybridized with an RNA molecule having a complementary sequence to the base sequence of SEQ ID NO: 33, SEQ ID NO: 34, or SEQ ID NO: 35. This RNA molecule is constituted of at least 30 bases, at least 31 bases, at least 32 bases, at least 33 bases, at least 34 bases, or at least 35 bases, and the full length can be 35 bases, 36 bases, 37 bases, or 40 bases. An RNA molecule having a sequence homologous to the base sequence of SEQ ID NO: 33, SEQ ID NO: 34, or SEQ ID NO: 35 can also be an RNA molecule having a base sequence in which 1 base to 5 bases, more preferably 1 base to 4 bases, still more preferably 1 base to 3 bases, and yet more preferably 1 base are substituted, inserted, or deleted for an RNA molecule having preferably the base sequence of SEQ ID NO: 33, SEQ ID NO: 34, or SEQ ID NO: 35. Especially, according to the below Examples, in an RNA molecule having the base sequence of SEQ ID NOS: 34 or 35, even when 4 bases at the 5′ end were deleted, binding to patulin was kept. Therefore, an RNA molecule of the present invention can be an RNA molecule having a base sequence in which, although not particularly limited, 1 base to 5 bases, preferably 1 base to 4 bases, more preferably 1 base to 3 bases, and still more preferably 1 base are deleted for an RNA molecule having the base sequence of SEQ ID NO: 33, SEQ ID NO: 34, or SEQ ID NO: 35. Substitution, insertion, or deletion of bases that can be performed in an RNA molecule of the present invention are preferably performed at the portion of 4 bases at the 5′ end, especially the 5′ end of the base sequence of SEQ ID NO: 33, SEQ ID NO: 34, or SEQ ID NO: 35.

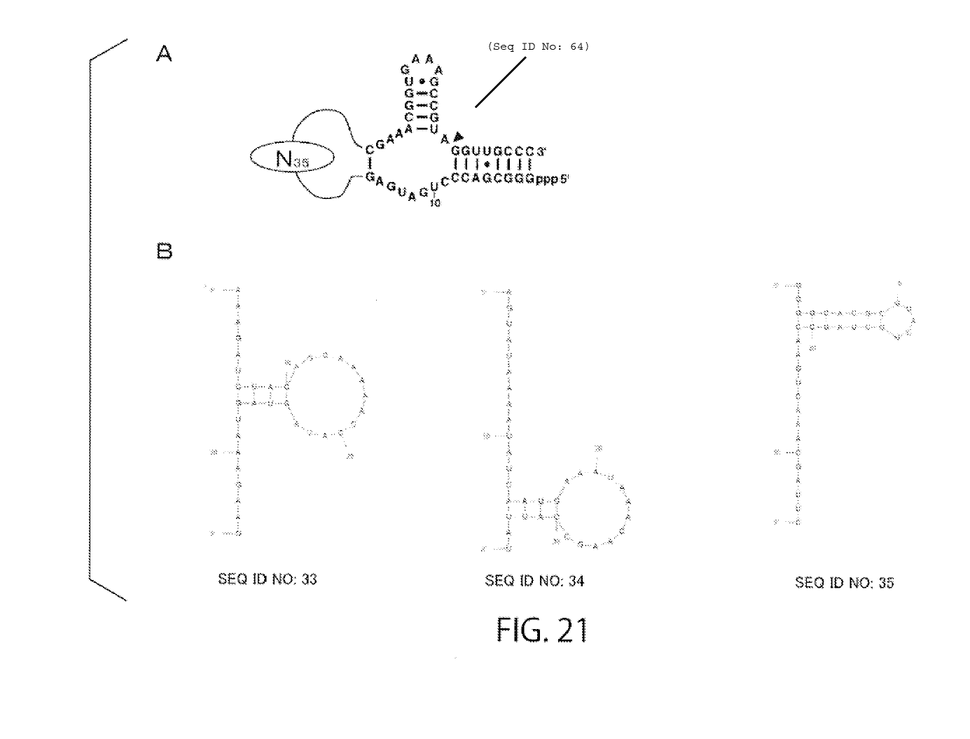

In order to ease the detection of binding to patulin, an RNA molecule of the present invention may be connected with a region detecting patulin. For example, an RNA molecule of the present invention can be an RNA construct for detection of patulin in combination with a self-cleaving ribozyme. In this case, the self-cleaving activity represents the binding of a self-cleaving ribozyme to patulin. An RNA construct of the present invention can be an RNA molecule of the present invention that is included in the molecule of a self-cleaving ribozyme, and for example, can be a molecule in which an RNA molecule of the present invention is inserted between the 16th G and 17th C of a self-cleaving ribozyme (sequence: 5′-GGGCGACCCUGAUGAGCGAAACGGUGAAAGCCGUAGGUUGCCC-3′; see FIG. 21A or Table 10).

An RNA construct of the present invention causes self-cleavage dependent on the binding to patulin. Therefore, an RNA construct of the present invention is an RNA construct that causes self-cleavage when bound to patulin. The binding of patulin to an RNA construct can be monitored by a change (e.g., increase) in self-cleaving activity of the RNA construct. The cleaving activity of an RNA construct can be detected as a change in the molecular weight of the RNA molecule by polyacrylamide gel electrophoresis (PAGE). In PAGE, the activity can be detected preferably by modified PAGE using polyacrylamide gel containing 8M urea. As used herein, a region showing patulin binding in such RNA construct can be referred to as patulin aptamer region or patulin RNA aptamer region.

In the present invention, a DNA molecule encoding an RNA molecule or an RNA construct of the present invention is provided.

There is no particular limitation on the self-cleaving ribozyme, and for example, a hammerhead ribozyme showing ligand-dependent self-cleaving activity is included (Makoto Koizumi et al., Nature Structural Biology (1999) 6: 1062-1071) (see FIG. 21A). As an RNA construct of the present invention, for example, a self-cleaving ribozyme of the present invention can be a hammerhead ribozyme having the base sequence of SEQ ID NOS: 30 to 32.

A DNA molecule of the present invention, for example, can be a DNA molecule having the base sequence of SEQ ID NO: 24, SEQ ID NO: 25, or SEQ ID NO: 26, or a DNA molecule having a sequence homologous to these base sequences. In this case, a DNA molecule of the present invention can be 25 nucleotides length to 35 nucleotides length, preferably 30 nucleotides length.

For example, a DNA molecule having a sequence homologous to the base sequence of SEQ ID NO: 24, SEQ ID NO: 25, or SEQ ID NO: 26 includes a DNA molecule having a base sequence showing 80% or more, 85% or more, 90% or more, or 95% or more sequence identity to the base sequence of SEQ ID NO: 24, SEQ ID NO: 25, or SEQ ID NO: 26, or a DNA molecule that is hybridized with a DNA molecule having a complementary sequence to the base sequence of SEQ ID NO: 24, SEQ ID NO: 25, or SEQ ID NO: 26. (This DNA molecule is constituted of at least 25 bases, at least 26 bases, at least 27 bases, at least 28 bases, at least 29 bases, or at least 30 bases, and the full length can be 30 bases, 31 bases, 32 bases, or 35 bases.) A DNA molecule having a sequence homologous to the base sequence of SEQ ID NO: 24, SEQ ID NO: 25, or SEQ ID NO: 26 can also be a DNA molecule having a base sequence in which 1 base to 5 bases, more preferably 1 base to 4 bases, still more preferably 1 base to 3 bases, and yet more preferably 1 base are substituted, inserted, or deleted for a DNA molecule having preferably the base sequence of SEQ ID NO: 24, SEQ ID NO: 25, or SEQ ID NO: 26. A DNA molecule of the present invention can be a DNA molecule in which, although not particularly limited, 1 base to 5 bases, preferably 1 base to 4 bases, more preferably 1 base to 3 bases, and still more preferably 1 base are deleted for a DNA molecule having the base sequence of SEQ ID NO: 24, SEQ ID NO: 25, or SEQ ID NO: 26. Deletion of bases can be performed, for example, at the end of a sequence (the 5′ end or the 3′ end).

The binding of a nucleic acid molecule of the present invention to ligands (e.g., patulin) can be easily detected, for example, using surface plasmon resonance (SPR). A method for detecting intermolecular bond using surface plasmon resonance is well known for persons skilled in the art.

In the present invention, in order to ease the detection, etc. of binding of a DNA molecule to ligands, for example, AMP or patulin, for example, the DNA molecule can be used by being connected with a region for detection of ligands, etc. (e.g., the effector region as mentioned below) and being incorporated into part of a DNA construct. In other words, in the present invention, a DNA construct including a DNA aptamer region (e.g., AMP aptamer region and patulin aptamer region) and an effector region that is activated by binding of ligands to the aptamer region is provided. In a certain aspect, a DNA construct of the present invention, for example, can be used by being incorporated as a patulin aptamer region into a DNA construct with the following constitution.

Further, using a DNA molecule, an RNA molecule, a nucleic acid molecule, or a nucleic acid construct of the present invention, a method for removing ligands (e.g., patulin) in a sample will be described.

Since a nucleic acid molecule of the present invention binds to ligands, a DNA molecule, an RNA molecule, a nucleic acid molecule, or a nucleic acid construct of the present invention can also be used for removing ligands from a sample. Therefore, in the present invention, a method for removing ligands from a sample using a DNA molecule, an RNA molecule, a nucleic acid molecule, or a nucleic acid construct of the present invention is provided. In other words, in the present invention, a method for removing ligands in a sample including binding of ligands to a DNA molecule, an RNA molecule, a nucleic acid molecule, or a nucleic acid construct of the present invention is provided.

In the present invention, removal of ligands from a sample can also be performed using a column on which a DNA molecule, an RNA molecule, a nucleic acid molecule, or a nucleic acid construct of the present invention is immobilized. Immobilization of a nucleic acid molecule in a column can be performed using well-known methods for persons skilled in the art.

More specifically, a method for removing ligands of the present invention can be a method including:

(a) obtaining a DNA molecule, an RNA molecule, a nucleic acid molecule, or a nucleic acid construct showing binding to ligands, preferably binding specificity to ligands,

(b) fabricating a ligand-adsorbing column by immobilizing the obtained DNA molecule, RNA molecule, nucleic acid molecule, or nucleic acid construct on the resin of a column, and

(c) adsorbing ligands to the column by contacting a sample with the obtained ligand-adsorbing column.

According to the present invention, a ligand-adsorbing column (e.g., patulin-adsorbing column) on which a DNA molecule, an RNA molecule, a nucleic acid molecule, or a nucleic acid construct of the present invention is immobilized is provided.

### Examples

### Example A1

### Design of DNA Aptamer for Detection of Compounds

In this example, a highly sensitive DNA molecule that can be used for detection of compounds was designed. Using a hairpin-loop-structured DNA molecule having the sequence of SEQ ID NO: 20 and including an adenosine monophosphate (AMP) aptamer and a redox DNAzyme as a base, a highly sensitive DNA molecule was designed by modifying its sequence.

Design (modification) of the DNA molecule was performed as follows. First, in all of Examples A1 to A6 below, only the aptamer mask sequence and the sequence in the junction region of the DNA molecule were modified. In the design, a DNA molecule was designed by setting conditions that the aptamer mask sequence is 3 or 4 bases length (M=3 or 4), that the DNA aptamer region that is hybridized with the aptamer mask sequence is 5′-AAGG-3′, and that the junction region is 1 to 5 bases length (J=1 to 5), and using the fact that it is predicted that a hairpin-loop-structured secondary structure is formed in the absence of AMP when predicted at a prediction condition that folding temperature is 37° C., Na⁺ concentration is 1 M, and Mg²⁺ concentration is 0 M, using a DNA secondary structure prediction program (UNAfold Version 3.8 that is provided without charge by University at Albany, The State University of New York (http://mfold.rna.albany.edu)) (e.g., it is predicted that the secondary structure shown in FIG. 1E is formed) as a guideline. For software package, a program for 32-bit Linux (trademark) was used. Secondary structure prediction was run using a command “UNAfold.pl --NA=DNA input fasta formatted file (name of the input file)” (in the prediction, 1 or more internal loop or bulge loop might be included in the aptamer mask region and the junction region). Specifically, as a guideline, a DNA molecule that is predicted to form a structure in which 4 bases at the 3′ end of the DNA aptamer are hybridized with 3 or 4 bases (aptamer mask region) adjacent to the 5′ side of the DNA aptamer in the absence of ligands is exhaustively designed. When the obtained sequences were counted, the relationship between the number of DNA sequences meeting the above mentioned conditions and M and J was as shown in Table 2.

TABLE 2

| | J = 1 | J = 2 | J = 3 | J = 4 | J = 5 |
|---|---|---|---|---|---|
| M = 3 | 3 | 14 | 112 | 96 | 96 |
| M = 4 | 55 | 220 | 2,452 | 1,802 | 2,932 |

At this time, the aptamer mask sequence or the junction region was not necessary to form a complete base pair to be hybridized, and an internal loop or a bulge loop might be included in the hybridized region. In all of the following Examples A1 to A6, modification of sequence was performed only in the aptamer mask region, part of the DNA aptamer region masked by the aptamer mask region, the junction region 1, and the junction region 2, and the sequence in the other regions was the same as that of a DNA sensor having the sequence of SEQ ID NO: 20.

### Example A2

### Construction of Screening System

In this example, construction of a system that can massively and simply screen DNA molecules was attempted.

With regard to a screening system, a system that electrochemically detects the activation of a DNAzyme was used. For electrochemical detection, use of an electrochemical detection microarray (CustomArray Inc., ElectraSense 12k microarray, product number: 1000081) and a detector (CustomArray Inc., ElectraSense detector, product number: 610036) was considered.

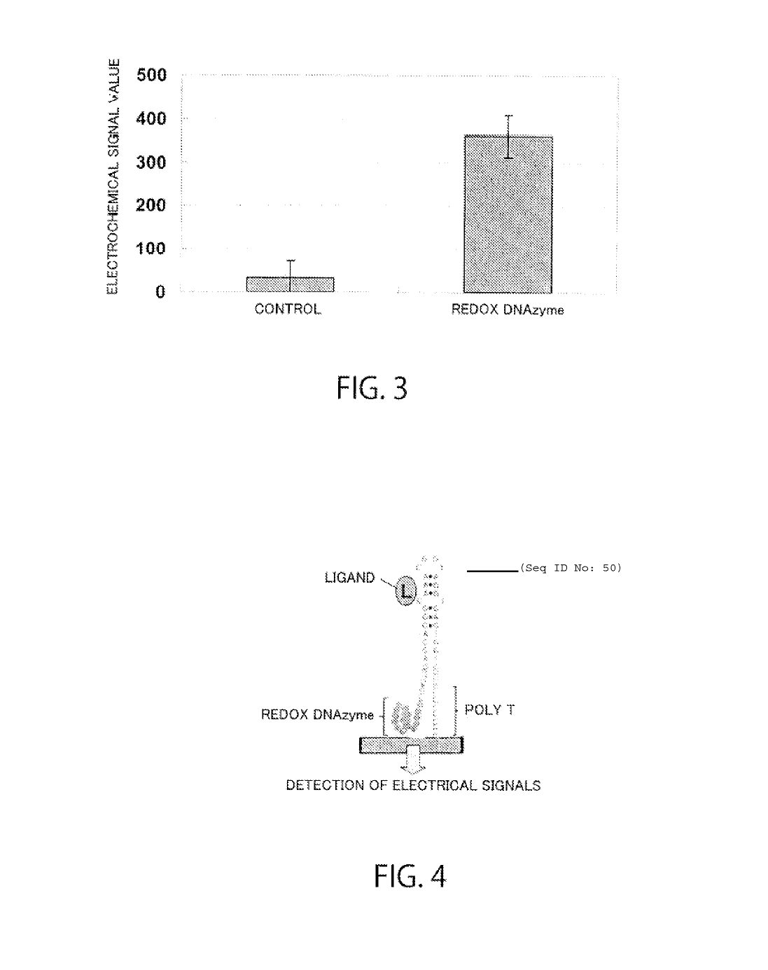

First, a redox DNAzyme (SEQ ID NO: 16; GGGTAGGGCGGGTTGGG) and a DNA without activity as a control (AATACGACTCACTATAGGAAGAGATGG) were synthesized on a microarray tip, and whether DNAzyme activity can be detected or not was investigated. In order to fix the 3′ end of these DNAs on an array, a poly T sequence of 51 bases was added to the 3′ end of a DNA to be fixed on an array. Five millimolar ABTS and 5 mM H₂O₂ were used. As a reaction buffer, 25 mM HEPES (pH 7.4), 20 mM KCl, 200 mM NaCl, and 1% DMSO were used. Heroin was added at a final concentration of 2.4 μM, and 2,2′-azino-bis(3-ethylbenzothiazoline-6-sulfonic acid) (ABTS) was added at a final concentration of 5 mM. Signal was measured under a temperature condition of 25° C. 1 minute after addition of H₂O₂ at a final concentration of 5 mM. Then, as shown in FIG. 3, activity of a DNAzyme can be detected with an electrochemical detection method.

The above electrochemical detection microarray can synthesize up to 12,000 types of DNAs on a tip, with the phosphoramidite method by signal control of a semiconductor electrode. Therefore, it was suggested that up to 12,000 types of DNA molecules can be screened at one time using an electrochemical detection microarray.

### Example A3

### Primary Screening

To confirm a rough relationship between the sequence and the detection sensitivity, in the first step screening, part of the designed sequences were screened.

First, since the number of the candidate sequences obtained by Example A1 was enormous, the DNA molecules designed in Example A1 for screening were sorted based on the free energy (dG) (kcal/mol) of the whole DNA molecule calculated by secondary structure estimation (i.e., difference in free energy between before folding and after secondary structure formation), and were subjected to screening. Specifically, for the DNA sequences showing the same dG, only one of them was randomly selected to be subjected to screening, and sorting was performed based on dG. The relationship between the number of DNA sequences subjected to screening and M and J was as shown in Table 3.

TABLE 3

| | J = 1 | J = 2 | J = 3 | J = 4 | J = 5 |
|---|---|---|---|---|---|
| M = 3 | 2 | 7 | 54 | 62 | 96 |
| M = 4 | 39 | 132 | 698 | 718 | 912 |

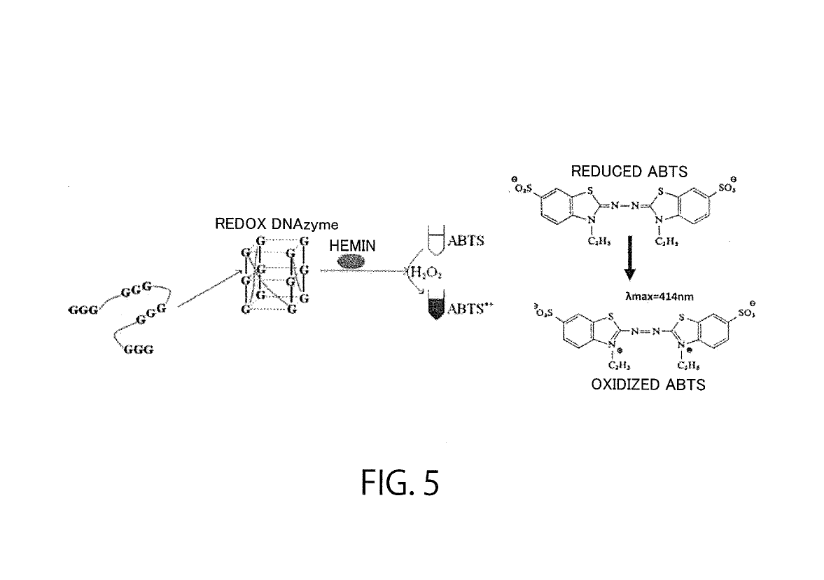

Further, a DNA molecule having the sorted base sequence was synthesized on this microarray. In the synthesis, a poly T sequence of 15 bases as a linker was added to the 3′ end of a DNA molecule, as mentioned above (FIG. 4). In this DNA molecule, only when AMP binds to the DNA aptamer region, a DNAzyme having the base sequence of SEQ ID NO: 16 becomes in the free state to form a complex with heroin, enabling oxidation of ABTS and reduction of H₂O₂ (FIG. 5).

Without changing the measurement conditions such as buffer condition, substrate concentration, and temperature condition from those in Example A2, only AMP concentration of 0 mM or 5 mM was added, each molecule at the concentration was incubated at room temperature for 30 minutes, and then AMP-concentration dependency of the DNAzyme activity of each molecule was confirmed. Measurement under the condition in which AMP of 5 mM was added was repeated 2 times, and measurement under the condition without the addition was performed once. The degree of the activation of the effector region of a DNA molecule was evaluated as a signal ratio of measurement signal by dividing the mean of 2 measurements when AMP of 5 mM was added by the value of measurement without the addition.

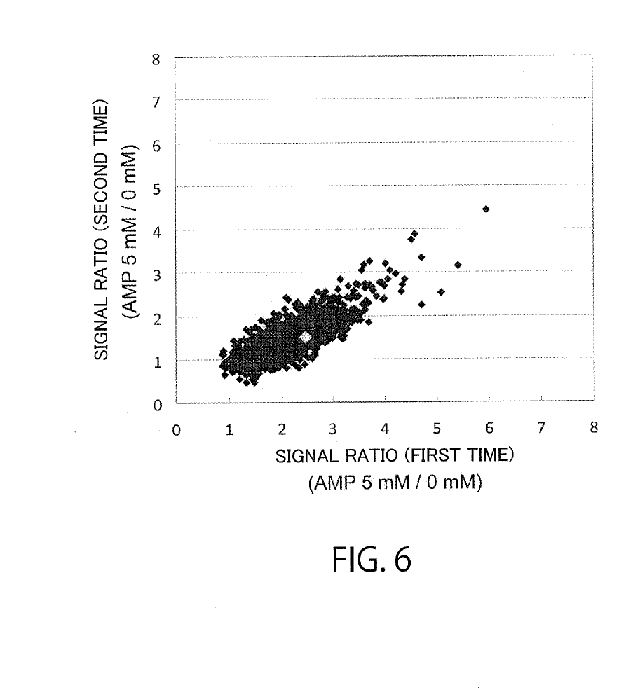

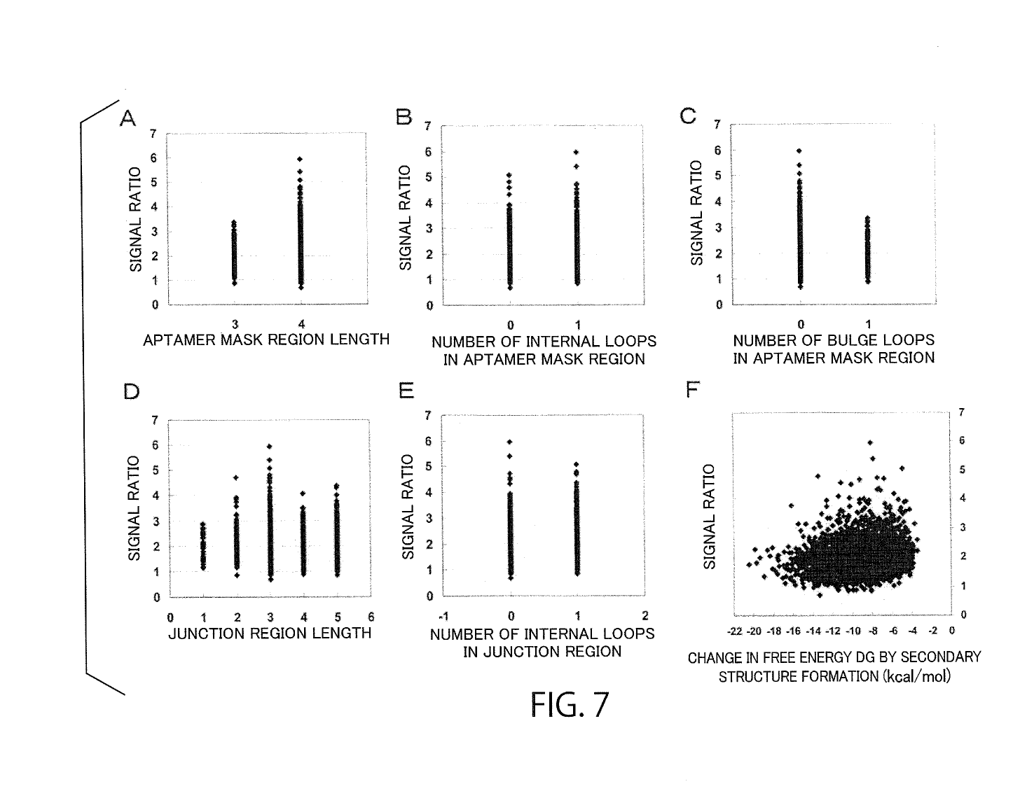

A signal ratio, as shown in FIG. 6, showed high reproducibility in the 2 repeated experiments. From this, electrochemical measurement was found to be a measurement method with high reproducibility. In addition, in order to examine the relationship between the base sequence of a DNA molecule and signal ratio, the relationship between the length, the number of internal loops, and the number of bulge loops, and signal ratio for each of the aptamer mask sequence and the junction region was examined and was made into a graph (FIG. 7). Then, as shown in FIG. 7A, it was revealed that the length of the aptamer mask region is preferably 4 bases length and the number of internal loops and the number of bulge loops are preferably 1 and 0, respectively (FIGS. 7B and C). As shown in FIG. 7D, it was revealed that the length of the junction region is most preferably 3 bases length. The number of internal loops of the junction region showed almost no differences between 0 and 1 (FIG. 7E).

The free energy (dG) of the whole DNA molecule by secondary structure formation (i.e., difference in free energy between before folding and after secondary structure formation) was calculated with the UNAfold, and the relationship between dG and the obtained signal ratio was made into a graph (FIG. 7F). Then, there was a tendency that a smaller dG value is associated with a smaller signal ratio. A high signal ratio was observed when dG was −14 to −4 kcal/mol, and an especially high signal ratio was observed when dG was −8 to −6 kcal/mol. This result suggested that dG of a DNA molecule within a certain range is important for highly sensitive detection of ligands by a DNA molecule.

Furthermore, for 6 types of DNA molecules that showed the highest signal ratio by the above mentioned screening (i.e., DNA molecules for the most highly sensitive detection of AMP), the activity was further verified by another method and the sequence was examined. Specifically, since ABTS shows absorption at 414 nm when oxidized by a DNAzyme, by examining the change in absorbance by an absorbance measurement method, the amount of oxidized ABTS can be determined, and thus the degree of the activation of a DNAzyme can be evaluated. Then, to a measurement buffer (25 mM HEPES (pH 7.1), 10 mM NaCl), each of 6 types of DNA was added so that the concentration was 12.5 μM, and heroin and ABTS were added so that the concentration was 0.5 μM and 5 mM, respectively. In addition, AMP was added so that the concentration was 0 mM, 0.05 mM, 0.5 mM, 2.5 mM, or 5.0 mM. Absorption was measured 5 minutes after the initiation of reaction by adding H₂O₂ at a final concentration of 5 mM. Measurement was performed 3 times. The absorbance change of ABTS at 414 nm ΔA_{414 nm} was calculated by ΔA_{414 nm}=[mean absorbance when AMP was added]−[mean absorbance when AMP was not added]−[mean absorbance when AMP was added and DNA was not added].

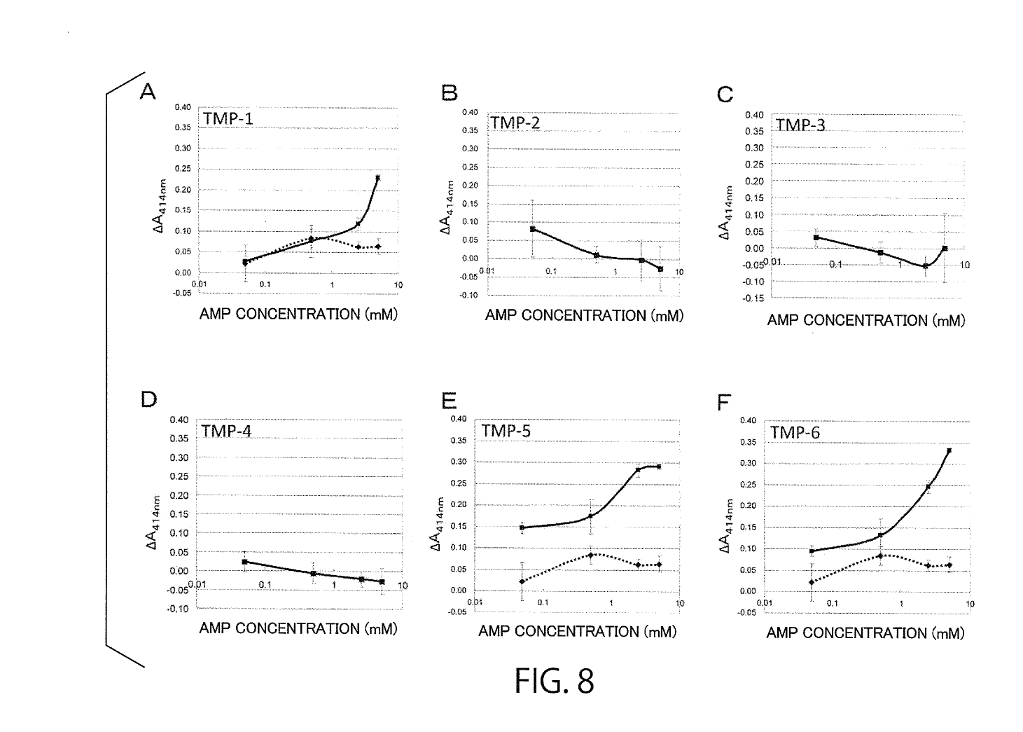

The results were as shown in FIG. 8. In other words, of 6 types of DNA molecules (each is referred to as TMP-1 to 6), TMP-1, 5, and 6 showed an AMP-concentration-dependent increase in the activity of a DNAzyme even by absorbance measurement. On the other hand, TMP-2 to 4 showed a high signal ratio by screening, while they showed no AMP-concentration-dependent increase in the activity of a DNAzyme by absorbance measurement.

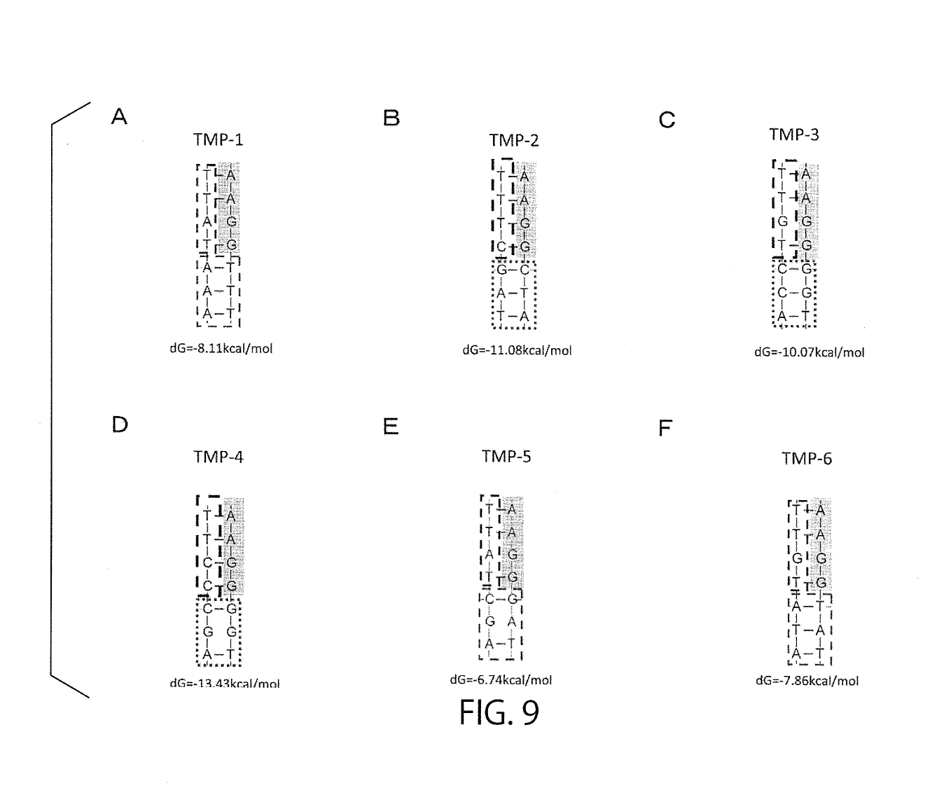

The secondary structure and the DNA sequence of the aptamer mask region and the junction region predicted in TMP-1 to 6 were as shown in FIG. 9. In other words, all of TMP-1, 5, and 6 that showed an AMP-concentration-dependent increase in the activity of a DNAzyme were predicted to form 2 T-A base pairs, 1 internal loop, and 1 T-G mismatched base pair in the aptamer mask region in the absence of AMP. Furthermore, the length of the aptamer mask region was 4 bases length for all of them, and the length of the junction region was 3 bases length for all of them. The free energy (dG) when a base pair was not formed and when a base pair was formed calculated by secondary structure estimation was −10 kcal/mol or more for all of TMP-1, 5, and 6. The aptamer mask region and the junction region of the DNA molecule of all of TMP-1, 5, and 6 did not form complete hybridization (such that all bases across the full length form a base pair) and included an internal loop or a bulge loop in the absence of AMP. For TMP-1 and 6 that showed an AMP-concentration-dependent increase in the activity of a DNAzyme, all sequences in the junction region formed an A-T base pair in the absence of AMP.

Especially, although it has the same aptamer mask sequence as that of TMP-1, 5, and 6, TMP-3 showed no an AMP-concentration-dependent increase in the activity of a DNAzyme. The dG of TMP-3 was −10.07 kcal/mol, and it is considered that TMP-3 could not cause a structural change at the time of binding of AMP because the junction region formed 2 G-C base pairs and the secondary structure was too stabilized (i.e., dG was too small). In other words, this result suggested that dG is preferably a constant value or more and that the number of G-C base pairs in the junction region is preferably 0 or 1

It was found that in TMP-2 to 4 that showed no AMP-concentration-dependent increase in the activity of a DNAzyme, the free energy (dG) calculated by secondary structure estimation was smaller than −10 kcal/mol for all of them, and the secondary structure in the absence of AMP was highly stabilized. In other words, it was suggested that in order to show activity by a DNAzyme, base pairs in the aptamer mask region and the junction region need to be removed with binding of AMP to the AMP aptamer, and that these regions should not be too stabilized by hybridization. In TMP-2 and 4 that showed no AMP-concentration-dependent increase in the activity of a DNAzyme, all bases in the aptamer mask region formed a base pair in the absence of AMP, and in TMP-2, furthermore, all bases in the junction region formed a base pair and all bases across the full length in the junction region formed a base pair in the absence of AMP. This suggested that preferably at least 1 internal loop or bulge loop is formed in the absence of AMP in either the aptamer mask region or the junction region.

Thus, it was suggested that when the aptamer mask region and the junction region that connect the AMP aptamer region with the DNAzyme region have a constant length and have a constant rule as mentioned above, a DNA molecule functions as a highly sensitive sensor showing an AMP-concentration-dependent increase in the activity of a DNAzyme. It was suggested that when the free energy (dG) calculated by secondary structure estimation is too large, the structure becomes unstable and a basic secondary structure to exert a function as a DNA sensor (e.g., the structure of the formula (I)) becomes unlikely to be formed, and when dG is too small, the structure is too stable and structural change of the secondary structure is expected to be unlikely to occur when AMP binds to a molecule, and thus dG of a DNA molecule is preferably −10 to −6.5 kcal/mol.

When the sensitivity as an AMP sensor of a known DNA sensor having the sequence of SEQ ID NO: 20 was compared with that of TMP-5 obtained in this example by absorbance measurement, as shown in FIGS. 8A, E, and F, the absorbance change was about 0.1 for the known DNA sensor, while the absorbance change was larger for TMP-5 with a value of 0.3, showing that TMP-5 is superior in the sensitivity as a sensor. The dynamic range was about 1 order (about 0.05 mM to about 0.5 mM) for the known DNA sensor, while it was about 3 order (about 0.005 mM to about 5 mM) for TMP-5, and thus TMP-5 had a wide dynamic range in which it can be used as a sensor in a wide concentration range. Thus, TMP-5 was superior to the known DNA sensor having the sequence of SEQ ID NO: 20 in both sensitivity as a sensor and dynamic range. Similar to TMP-5, TMP-1 and 6 had a high sensitivity and a wide dynamic range. In the known DNA sensor having the sequence of SEQ ID NO: 20, the free energy dG when a secondary structure is formed in the absence of AMP was calculated to be −12.79 kcal/mol.

These results revealed that the screening system constructed in Example A2 is also useful in screening of a DNA molecule of the present invention that highly sensitively reacts to ligands to be activated.

### Example A4

### Secondary Screening

In the secondary screening, sequences meeting a condition in which the DNA sensor obtained from the primary screening is highly sensitive were exhaustively screened.

According to the primary screening, a DNA molecule for highly sensitive detection of AMP met a condition that (i) the length of the aptamer mask region is 4 bases length, (ii) the length of the junction region is 3 bases length, and (iii) the number of G-C base pairs in the junction region is up to 1. The (iv) dG when the aptamer mask region and the junction region form a secondary structure was calculated to be −10 kcal/mol or more.

Therefore, all of DNA molecules meeting the above mentioned (i) to (iv) conditions, and having, when a DNA secondary structure is predicted with the UNAfold, (v) a DNA sequence in which only a hairpin-loop-structured secondary structure is estimated in the absence of AMP were included in the secondary screening. (In addition to this, molecules in which the length of the aptamer mask region is 5 bases length were also included in the secondary screening.) In the prediction, 1 or more internal loop(s) or bulge loop(s) might be included in the aptamer mask region and the junction region. The relationship between the number of DNA sequences obtained and M and J was as shown in Table 4.

TABLE 4

| | J = 3 |
|---|---|
| M = 4 | 3,072 |
| M = 5 | 6,552 |

By adding a sequence in which a poly T sequence of 15 bases was added to the 3′ end of the obtained sequence, a DNA molecule was synthesized on an array similar to Example A2. Signal was measured similar to Example A2, and signal was measured when 5 mM of AMP was added and was not added to calculate a signal ratio.

As a result, 55 DNA molecules showing a larger signal ratio than that of TMP-5 obtained in Example A3 were found. Each of these 55 DNA molecules was further verified by absorbance measurement similar to Example A3.

Similar to TMP-1, 5, and 6 that showed an AMP-concentration-dependent increase in the activity of a DNAzyme, 7 of 55 DNA molecules were predicted to form 2 T-A base pairs, 1 internal loop, and 1 T-G mismatched base pair in the aptamer mask region (FIG. 10). Of these 7 DNA molecules, 2 DNA molecules in which dG was calculated to be less than −6.5 kcal/mol (FIGS. 10A and B) showed an AMP-concentration-dependent increase in the activity of a DNAzyme even by absorbance measurement, and 5 DNA molecules in which dG was calculated to be −6.5 kcal/mol or more showed no AMP-concentration-dependent increase in the activity of a DNAzyme (FIGS. 10C to G).

Absorbance measurement of 48 DNA molecules showing a larger signal ratio than that of TMP-5 showed an AMP-concentration-dependent increase in the activity of a DNAzyme in 6 of these DNA molecules (FIG. 11). In these 6 DNA molecules, dG for all of the DNA molecules was −6.0 kcal/mol or less, and 4 DNA molecules in which dG was calculated to be −6.5 kcal/mol or less showed an especially highly sensitive AMP-concentration-dependent increase in the activity of a DNAzyme (FIGS. 11A, C, D, and F). In these 6 DNA molecules, DNA molecules forming 2 base pairs and 1 T-G mismatched pair (FIGS. 11A and B), 3 base pairs (FIGS. 11C and D), and 4 base pairs (FIGS. 11E and F) in the aptamer mask region were observed. In the all cases, the aptamer mask region had either of an internal loop or a bulge loop.

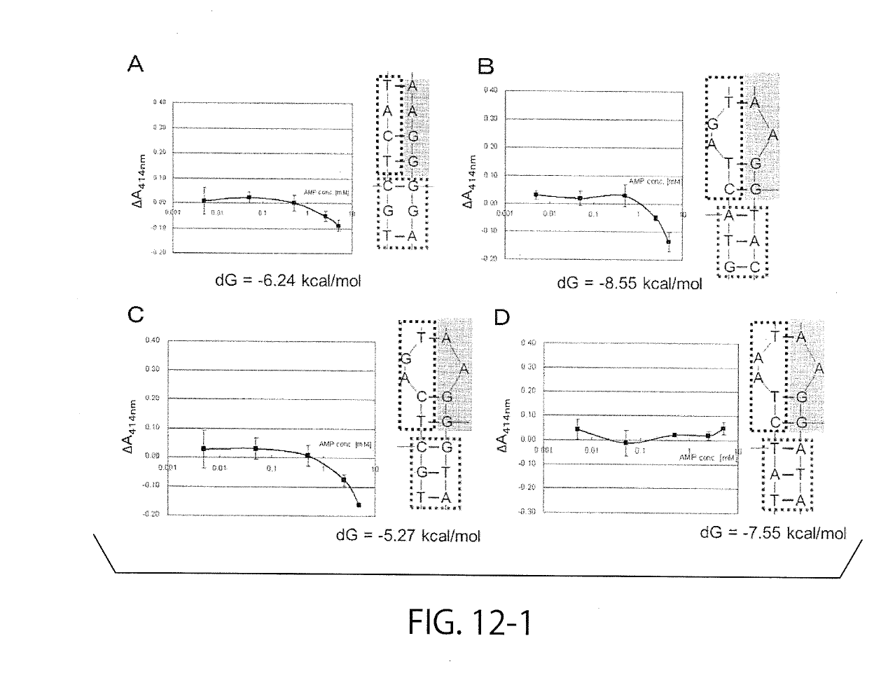

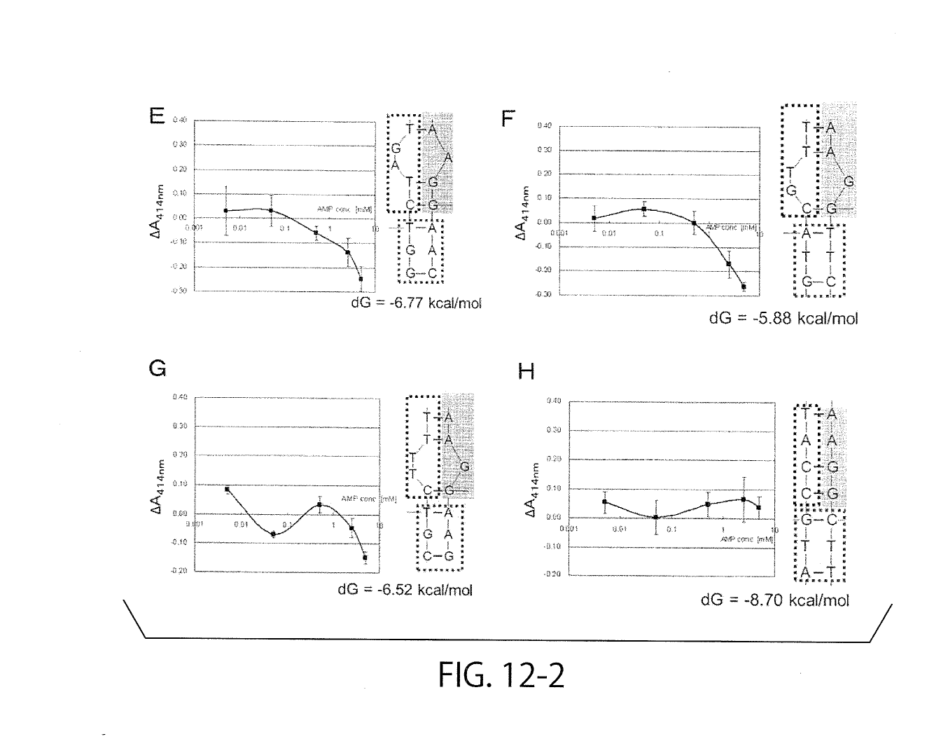

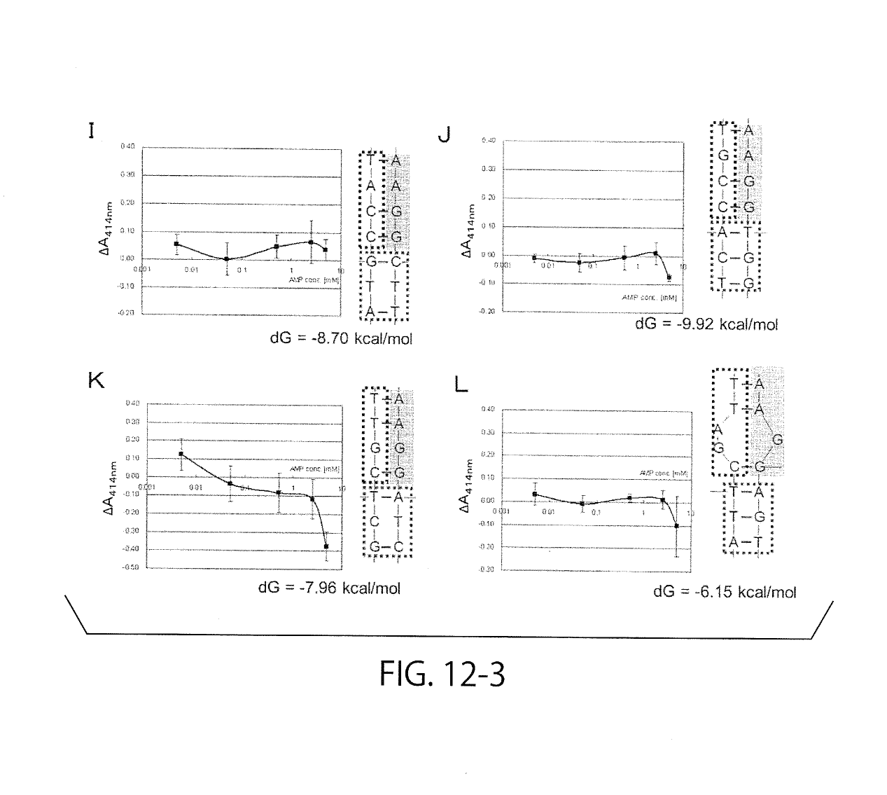

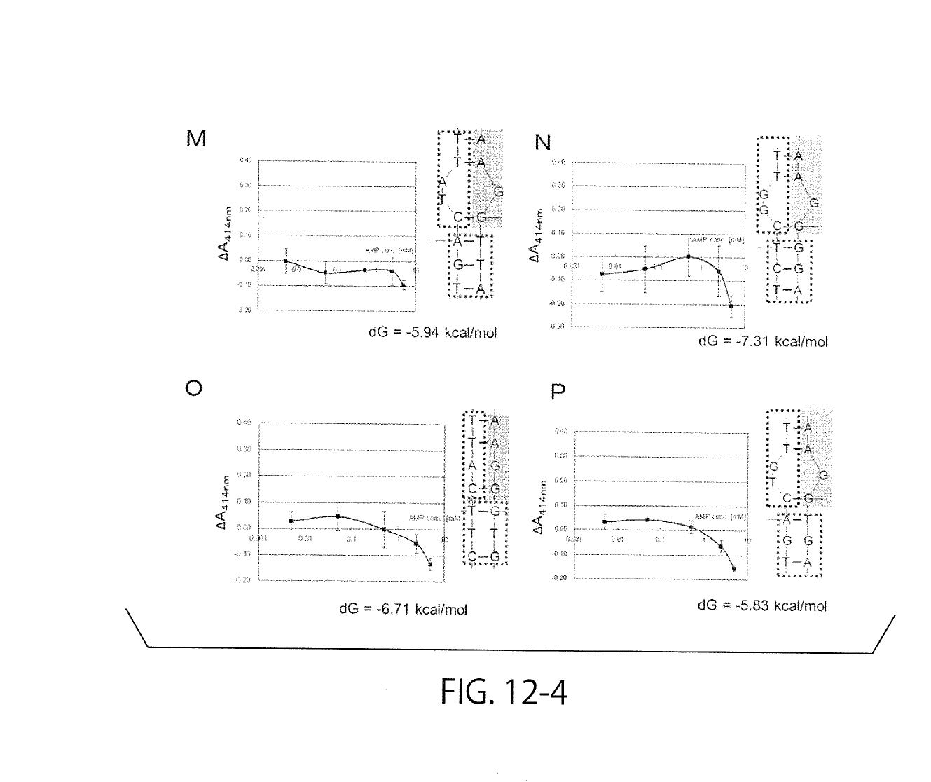

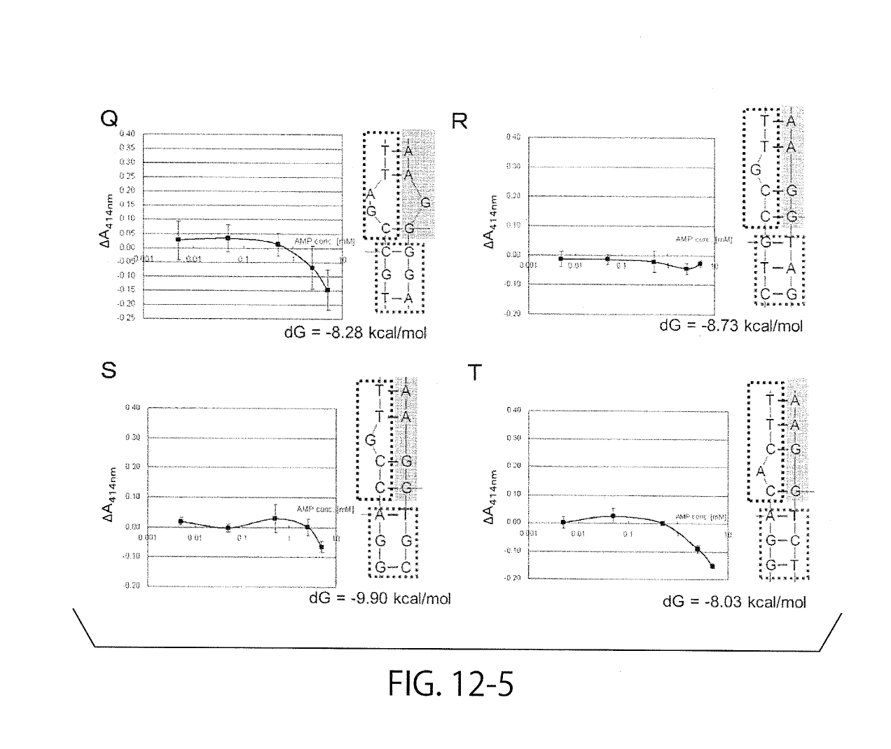

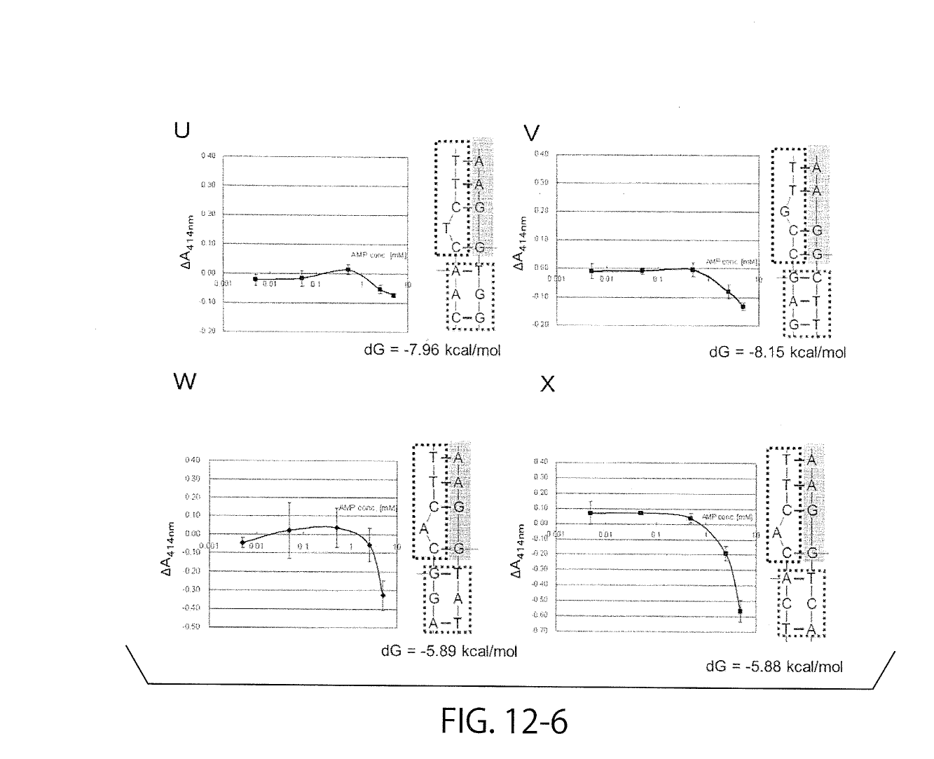

Of the above mentioned 55 DNA molecules, even in DNA molecules having the same aptamer mask sequence as that of the above mentioned 6 DNA molecules showing an AMP-concentration-dependent increase in the activity of a DNAzyme, some DNA molecules showed no AMP-concentration-dependent increase in the activity of a DNAzyme (FIG. 12). Specifically, there were 5 DNA molecules forming 2 base pairs, 1 internal loop, and 1 T-G mismatched pair (FIGS. 12A to E), 12 DNA molecules forming 3 base pairs (FIGS. 12F to Q), and 7 DNA molecules forming 4 base pairs (FIGS. 12R to X).

In a DNA molecule that causes ligand-dependent activation of the effector region, the 3′ end of the DNA aptamer region that is hybridized with the aptamer mask region was 4 bases length in many cases, while when the aptamer mask region was 5 bases length, a DNA molecule of up to 7 bases was found (data not shown).

### Example A5

### Type of Mismatched Base Pair in Aptamer Mask Region and Junction Region

In this example, the effects of type of mismatched base pair on the sensitivity were evaluated by comparing the highly sensitive DNA molecules with low sensitive DNA molecules that were obtained by the primary screening (Example A3) and the secondary screening (Example A4).

Existence of mismatched base pair is known to affect the stability of DNA secondary structure (SantaLucia, J. Jr. and Hick, D. (2004) Annu. Rev. Biophys. Biom.). The effects of mismatched base pair on the stability of DNA secondary structure vary depending on the type of an adjacent base pair. Then, the effects of mismatched base pair on dG of a DNA molecule were examined for a highly sensitive sequence and a low sensitive sequence by absorbance measurement. The effects of mismatched base pair on dG (ddG) were evaluated with the method disclosed in SantaLucia, J. Jr. and Hick, D. (2004) Annu. Rev. Biophys. Biom., using the UNAfold Version 3.8. The examined DNA molecules were limited to molecules in which dG when the whole DNA molecule forms a secondary structure was −9 to −6 kcal/mol.

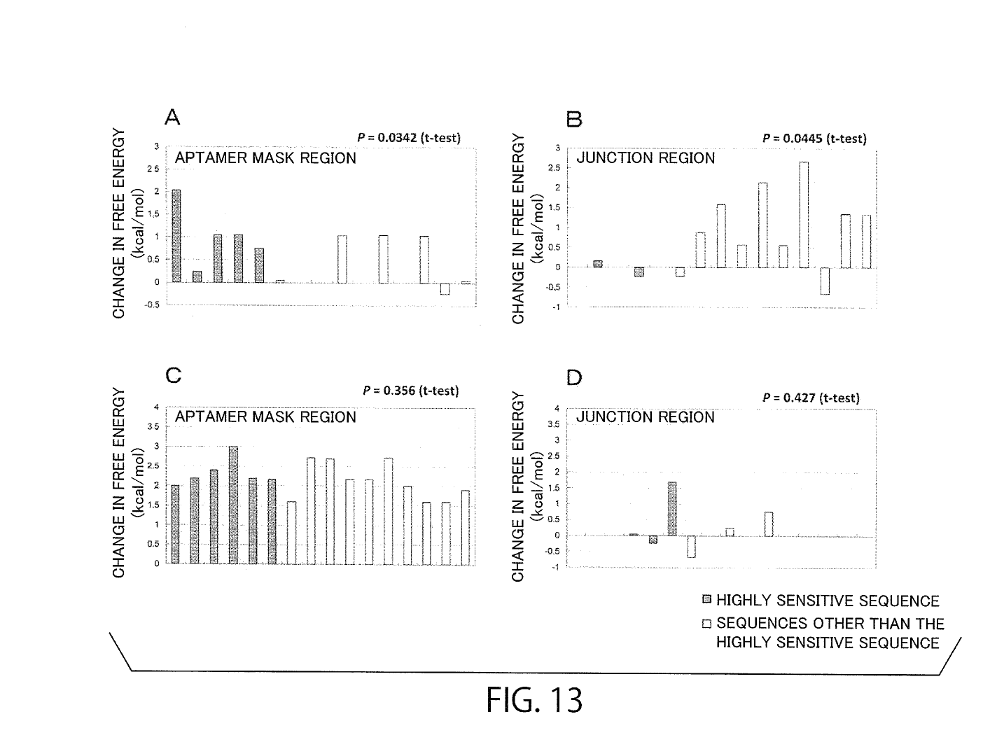

The results revealed that, as shown in FIG. 13, when the aptamer mask region is 4 bases length, the effects of mismatched base pair in the aptamer mask region on dG are preferably larger. A t-test showed statistically-significant differences between 5 DNA molecules that were confirmed to be highly sensitive by absorbance measurement and other 10 DNA molecules (FIG. 13A; p<0.05). It was found that when the aptamer mask region is 4 bases length, smaller effects of mismatched base pair in the junction region on dG were better (FIG. 13B; p<0.05).

In this way, it was suggested that the sensitivity of a DNA molecule varies depending on the type of mismatched base pair forming an internal loop.

Meanwhile, when the aptamer mask region is 5 bases length, there were no statistically-significant differences between the effects of mismatched base pair on dG and the detection sensitivity of a DNA molecule.

### Example A6

### Optimization of DNA Molecule as a Sensor

In this example, for TMP-5 obtained in Example A3 that showed a highly sensitive AMP-concentration-dependent increase in the activity of a DNAzyme, optimization of a sensor of the DNA molecule was attempted by modifying the sequence only in the aptamer mask region and the junction region.

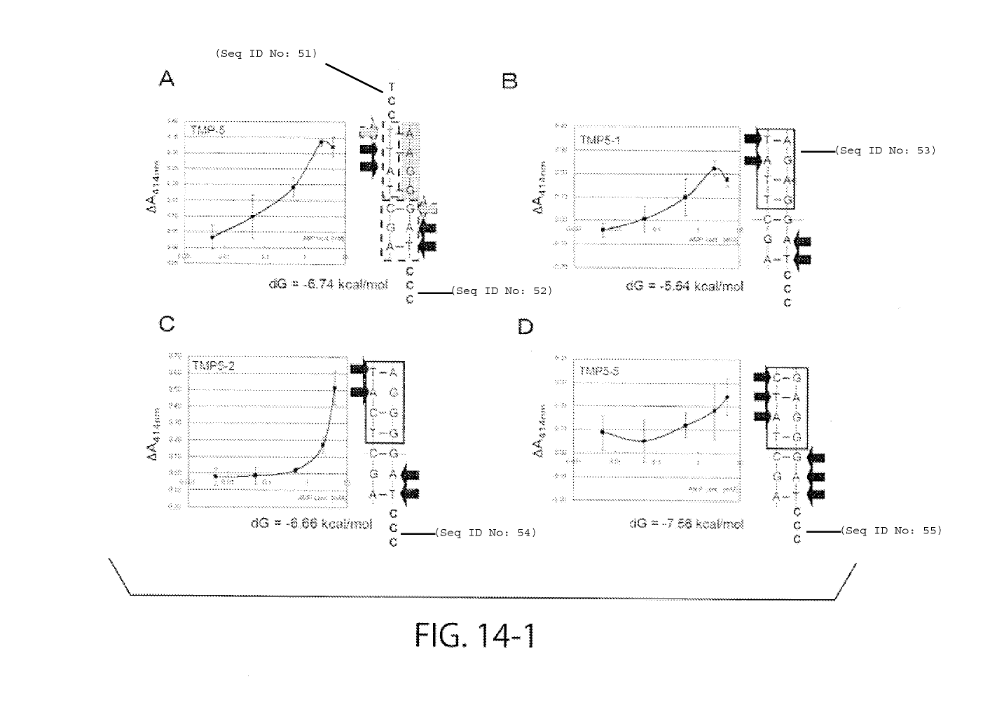

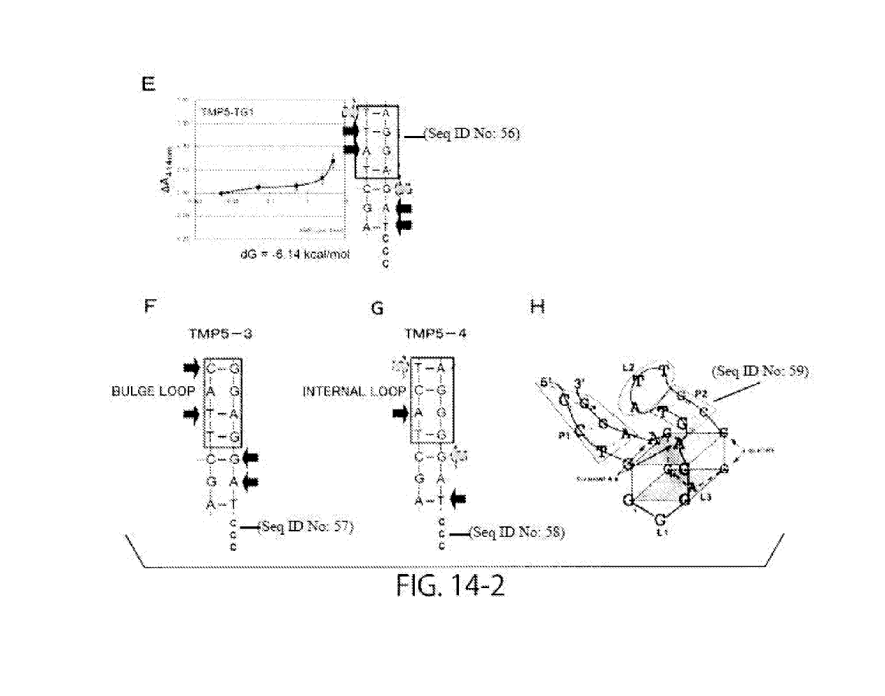

First, for the prepared TMP 5-1 to 5 and TG1, an AMP-concentration-dependent increase in the activity of a DNAzyme was confirmed by absorbance measurement (FIG. 14). Then, an AMP-concentration-dependent increase in the activity of a DNAzyme was observed for TMP 5-1, 2, and 5, and TG1 (FIGS. 14B to E), while TMP 5-3 and 4 (FIGS. 14F and G) showed almost no AMP-concentration-dependent increase in the activity of a DNAzyme (for TMP-5, see FIG. 14A).

A DNA sensor having the sequence of SEQ ID NO: 12 (TMP 5-1) is considered to form a conformation like FIG. 14H in the DNA aptamer region when AMP is bound. When bound to AMP, the aptamer mask region and the junction region 1 are considered to form a base pair with bases at the 3′ side of 4 bases (FIG. 14H) and be stabilized in this state, and as a result, the active state of a DNAzyme is considered to be kept. Then, for the prepared TMP 5-1 to 5 and TG1, a base pair formed at the time of binding of AMP was examined.

Investigation on hybridization of the aptamer mask region with the junction region 2 at the time of binding of AMP revealed that TMP 5-1, 2, 5, and TG1 that showed an AMP-concentration-dependent increase in the activity of a DNAzyme form 2 or more consecutive base pairs between the aptamer mask region and the junction region 2 (arrows in FIGS. 14A to E). TMP 5-3 and 4 that showed almost no AMP-concentration-dependent increase in the activity of a DNAzyme formed 2 base pairs across a bulge loop or an internal loop (FIGS. 14F and G). Like TMP 5-1 or 5-TG1, even in the case of dG of −6.5 kcal/mol or more (for TMP 5-1, dG of −6.0 kcal/mol or more), an AMP-concentration-dependent increase in the activity of a DNAzyme was shown at the time of binding of AMP when 2 or more consecutive base pairs are formed between the aptamer mask region and the junction region 2 (FIGS. 14B and E).

The results suggested that having a factor that stabilizes the structure after binding of AMP is important for showing an AMP-concentration-dependent increase in the activity of a

DNAzyme. It was also revealed that even when the secondary structure in the absence of AMP is somewhat unstable (e.g., even when dG is −6.5 to −5.0 kcal/mol), having a factor that stabilizes the structure after binding of AMP may show an AMP-concentration-dependent increase in the activity of a DNAzyme. It was revealed that forming 2 or more consecutive base pairs between the aptamer mask region and the junction region 2 at the time of binding of AMP is important as a specific factor that stabilizes the structure after binding of AMP.

### Example A7

### DNA Molecule for Detection of Arginine Aptamer

In this example, in order to verify the applicability to other than an AMP aptamer, the same experiment was performed by substituting the DNA aptamer region by the arginine aptamer.

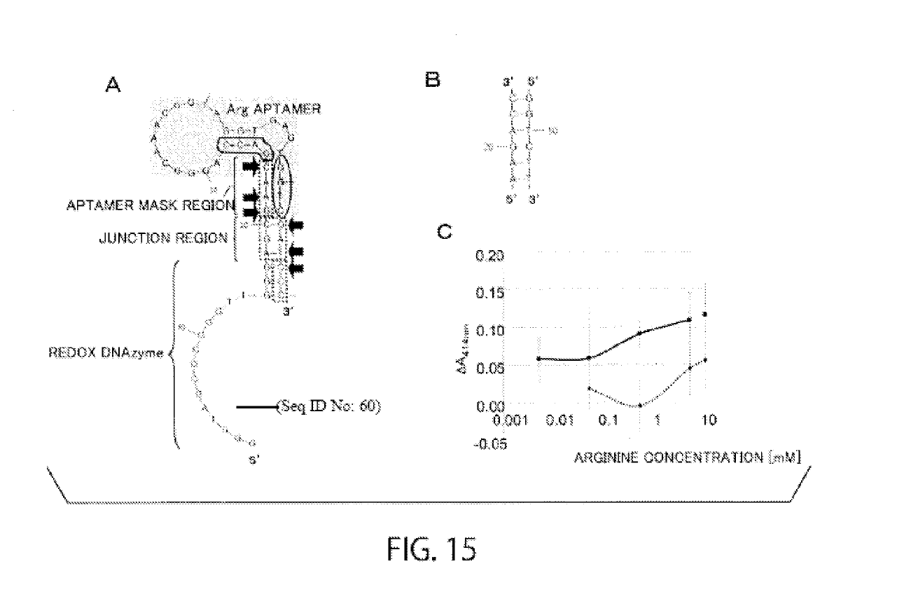

First, the length of the junction region was 3 bases length, and the sequence was the same as that of the junction region of TMP-5. Next, the aptamer mask region was designed so that it forms 3 base pairs and 1 internal loop between this region and 4 bases at the 3′ end of the arginine aptamer (TMP-5^{Arg} having the base sequence of SEQ ID NO: 19). According to the UNAfold, the designed DNA molecule was estimated to form the secondary structure shown in FIG. 15A. As shown in the arrows in FIG. 15A, the structure after binding of arginine was estimated to form 2 consecutive base pairs between the aptamer mask region and the junction region 2. As a control DNA molecule, of the DNA molecule in FIG. 15A, a DNA molecule in which the aptamer mask region forming 4 complete base pairs between this region and 4 bases at the 3′ end of the arginine aptamer and the length of the junction region was 2 bases length was used (FIG. 15B). A measurement condition was the same as in Example A3 except that AMP was substituted by arginine.

Then, as shown in FIG. 15C, TMP-5^{Arg} showed an absorption change 2-fold or more higher than that of the control at an arginine concentration of 10 mM. In this way, it was revealed that the results of Examples A1 to A5 using an AMP aptamer are universally established even when an arginine aptamer is used.

### Example B1

### Design of Patulin DNA Aptamer

In this example, obtainment of a patulin-binding DNA aptamer molecule was attempted.

Using the TMP-5 molecule obtained in Example A3, obtainment of a patulin-binding DNA aptamer molecule was attempted by modifying only the DNA aptamer region.

First, the patulin aptamer region was designed. Due to limitations of the electrochemical detection microarray (CustomArray Inc., ElectraSense 12k microarray, product number: 1000081) used in this example, samples that could be subjected to screening were limited to 12,000 samples, and thus the patulin aptamer was designed by establishing the following limiting conditions. In other words, a DNA molecule was designed by establishing the limiting conditions of (condition 1) the patulin aptamer region portion is 30 bases length, (condition 2) only 1 loop is formed in the absence of patulin, (condition 3) a loop is formed from the nucleotide of 3 to 7 bases length, and (condition 4) the number of base pairs formed in the stem portion is 6 to 9. By secondary structure prediction, sequences that are predicted to have a structure in which the aptamer mask region is hybridized with the 3′ end of the aptamer and the junction region 1 is hybridized with the junction region 2 were narrowed down, and the number of candidate molecules was about 12,000 types. The breakdown of the number of sequences of the candidate molecules synthesized on an array was as shown in Table 5.

TABLE 5
Number of base pairs and number of bases in loop in
secondary structure of molecule included in screening

| Number of | | | | | | |
|---|---|---|---|---|---|---|
| base pairs | Number of bases in loop (base) | | | | | |
| (base pair) | 3 | 4 | 5 | 6 | 7 | Total |
| 6 | 1 | 6 | 3 | 1 | 0 | 11 |
| 7 | 35 | 39 | 143 | 46 | 24 | 287 |
| 8 | 269 | 502 | 582 | 368 | 382 | 2,103 |
| 9 | 1,527 | 2,861 | 3,476 | 1,735 | 0 | 9,599 |
| Total | 1,832 | 3,408 | 4,204 | 2,150 | 406 | 12,000 |

### Example B2

### Obtainment of Patulin DNA Aptamer

In this example, obtainment of a patulin-binding DNA aptamer molecule was attempted.

Screening was performed with a system that electrochemically detects the activation of a DNAzyme. For electrochemical detection, an electrochemical detection microarray (CustomArray Inc., ElectraSense 12k microarray, product number: 1000081) and a detector (CustomArray Inc., ElectraSense detector, product number: 610036) were used.

The 12,000 types of DNA molecules designed were synthesized on this microarray so that 1 type of a molecule was in 1 spot. In the synthesis, a poly T sequence of 15 bases as a linker was added to the 3′ end of a DNA molecule, as mentioned above (e.g., see FIG. 4). As a reaction buffer, 25 mM HEPES (pH 7.4), 20 mM KCl, 200 mM NaCl, and 1% DMSO were used. After 5 mM and 100 μM of patulin was added to the reaction buffer, the molecule was incubated at room temperature for 30 minutes. Heroin was added at a final concentration of 2.4 μM, and 2,2′-azino-bis(3-ethylbenzothiazoline-6-sulfonic acid) (ABTS) was added at a final concentration of 5 mM. Signal was measured under a temperature condition of 25° C. 1 minute after addition of H₂O₂ at a final concentration of 5 mM. The electrical signal of each spot was measured in the presence and absence of patulin, and an electrical signal ratio (in the presence of patulin/in the absence of patulin) was calculated.

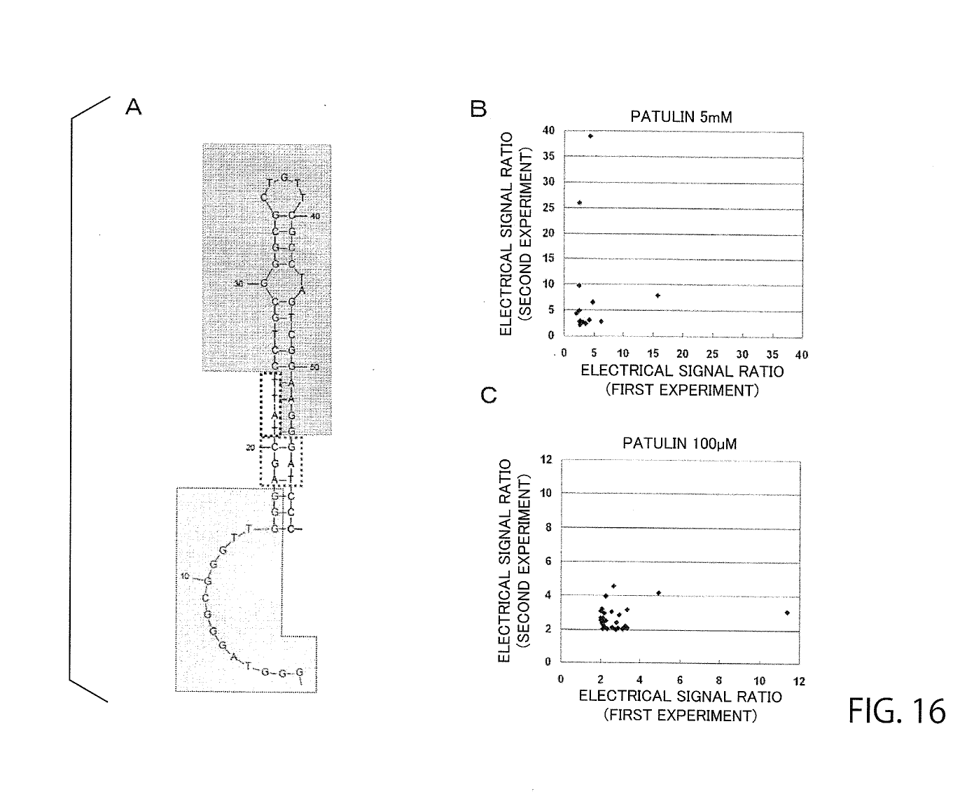

In FIG. 16, the representative example of the secondary structure of a TMP-5 molecule including the patulin aptamer region designed in Example B1 instead of the AMP aptamer region is shown (FIG. 16A), and electrical signal ratios for 14 sequences (patulin 5 mM, FIG. 16B) and 32 sequences (patulin 100 μM, FIG. 16C) showing the multiple change of a signal of 2-fold or more higher for both 2 probes are plotted. The reproducibility was investigated by a colorimetric test using ABTS for 6 sequences (candidate group 1 (First-Candidate)-1 to 1-6) which have 4-fold or more higher mean multiple change in 2 experiments at a patulin concentration of 5 mM among the sequences shown in FIG. 16B as a sequence with an electrical signal ratio of 2-fold or more higher, and for 12 sequences (candidate group 2 (Second-Candidate)-1 to 2-12) which have 3-fold or more higher mean multiple change in 2 experiments at a patulin concentration of 100 μM or 2 or more mean electrical signal ratio at a patulin concentration of 5 mM among of the sequences shown in FIG. 14C as a sequence with an electrical signal ratio of 2-fold or more higher.

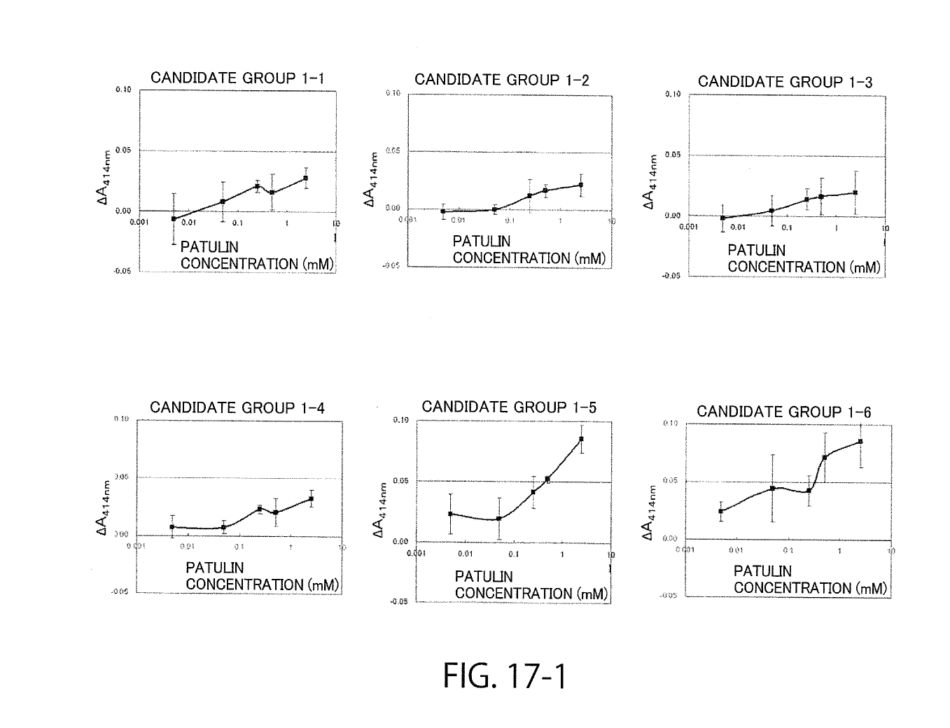

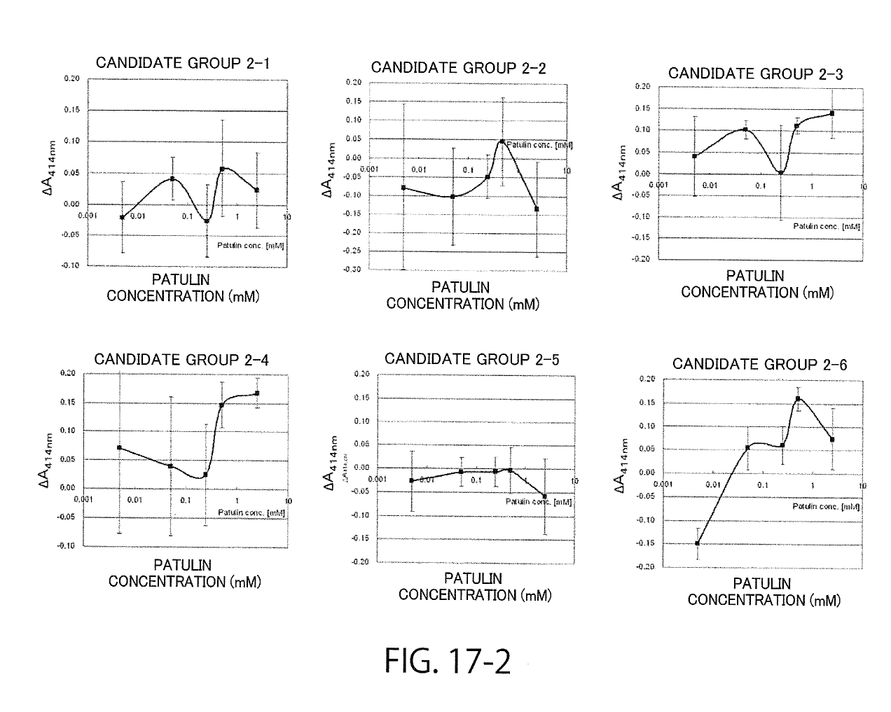

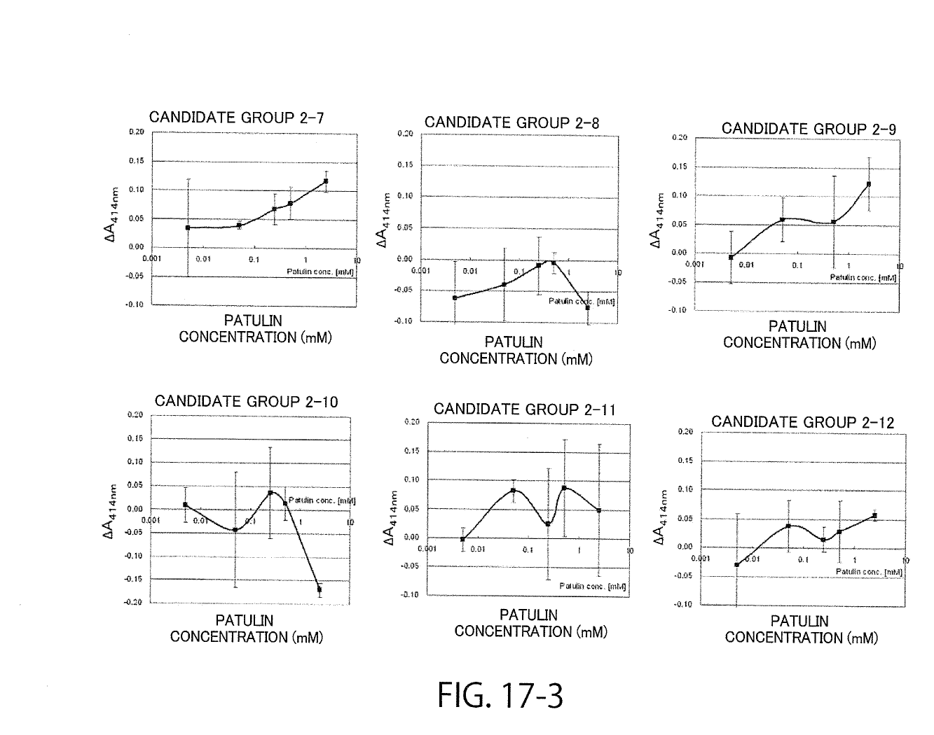

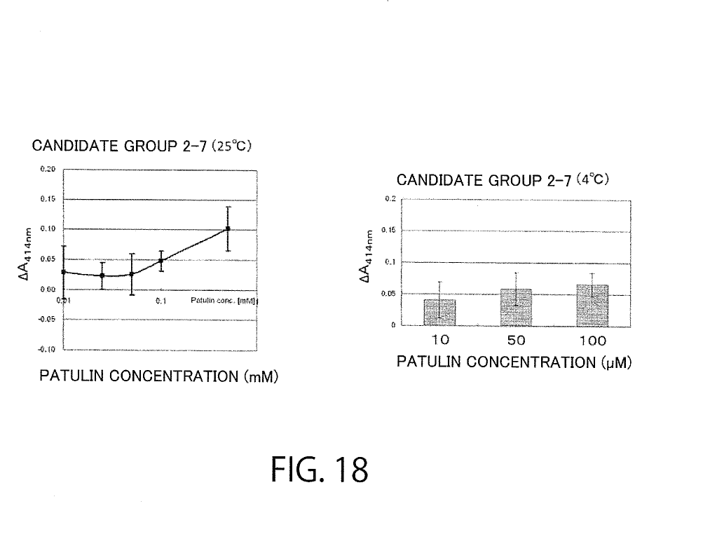

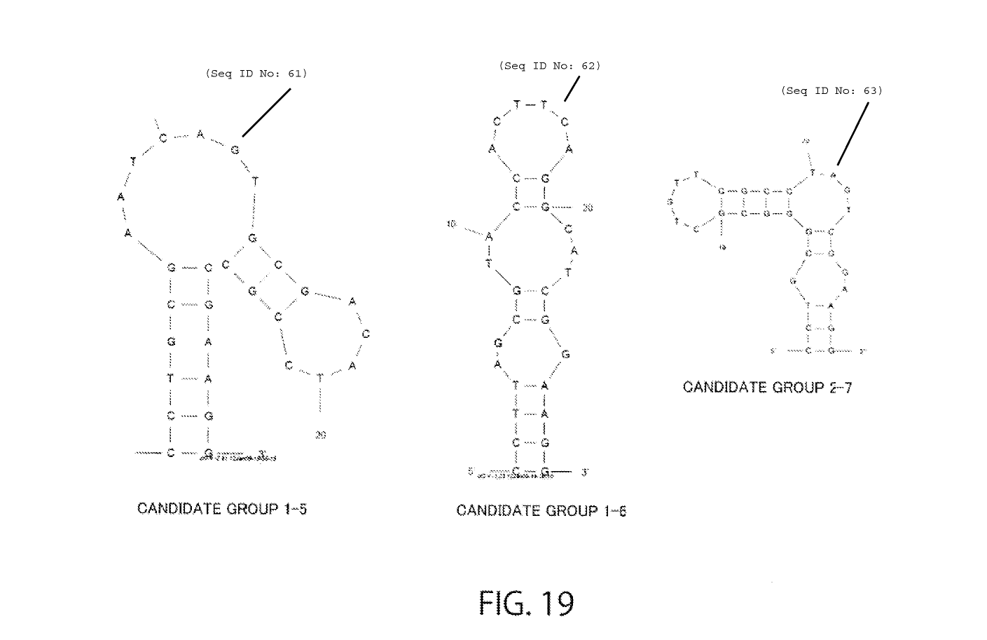

The results were as shown in FIG. 17. In other words, 2 groups (candidate group 1-5 and 1-6) of the DNA molecules of the candidate groups 1-1 to 1-6, and 1 group (candidate group 2-7) of the DNA molecules of the candidate group 2-1 to 2-12 showed a patulin concentration-dependent increase in the activity of a DNAzyme even by the colorimetric test. The measurement showed high reproducibility. For the candidate group 2-7, which seemed to be most highly sensitive, patulin was added so that the concentration was 0 mM, 0.01 mM, 0.025 mM, 0.05 mM, 0.1 mM, or 0.5 mM, and the detection sensitivity was investigated at a lower concentration side (FIG. 18). Similar investigation at 4° C. revealed that the measurement error becomes small and patulin can be further highly sensitively detected (FIG. 18). As a result, it was found that the candidate group 2-7 can detect patulin with a concentration of 10 μM. When the candidate groups 1-5 and 1-6 were investigated, these molecules could detect patulin of several hundred micromolars order (data not shown). The estimation results of the secondary structure of the patulin aptamer region of each DNA molecule are shown in FIG. 19. The candidate group 2-7 may be hereinafter referred to as SC-7.

The base sequence of these molecules was as shown in Table 6.

TABLE 6
Table 6: Base sequence of 3 types of DNA
constructs that showed high sensitivity for
patulin

| Se- | | SEQ |
|---|---|---|
| quence | Base sequence of full-length DNA | ID |
| name | molecule | NO |
| First- | 5′- | 21 |
| Candi- | GGGTAGGGCGGGTTGGGAGCTATTCCTGCGAATCAGTG | |
| date 5 | CGACATCCGCCGAAGGGATCCC-3′ | |
| First- | 5′- | 22 |
| Candi- | GGGTAGGGCGGGTTGGGAGCTATTCCTTAGCGTACCAC | |
| date 6 | TTCAGGCATCGGAAGGGATCCC-3′ | |
| Second- | 5′- | 23 |
| Candi- | GGGTAGGGCGGGTTGGGAGCTATTCCTGCGGGCGCTGT | |
| date 7 | TCGCCTAGTCGGAAGGGATCCC-3′ | |

*The underlined portion in the base sequence represents the patulin aptaner region in a DNA construct.

The base sequence in the patulin aptamer region portion of these molecules was as shown in Table 7.

TABLE 7
Table 7: Base sequence in the patulin
aptamer region of 3 types of DNA
constructs that showed high
sensitivity for patulin

| | Base sequence of the patulin | SEQ |
|---|---|---|
| Sequence | aptamer region portion of | ID |
| name | each sequence | NO |
| Patulin | 5′- | 24 |
| aptamer | CCTGCGAATCAGTGCGACATCCGCCGAAGG-3′ | |
| region of | | |
| First- | | |
| Candidate 5 | | |
| Patulin | 5′- | 25 |
| aptamer | CCTTAGCGTACCACTTCAGGCATCGGAAGG-3′ | |
| region of | | |
| First- | | |
| Candidate 6 | | |
| Patulin | 5′- | 26 |
| aptamer | CCTGCGGGCGCTGTTCGCCTAGTCGGAAGG-3′ | |
| region of | | |
| Second- | | |
| Candidate 7 | | |

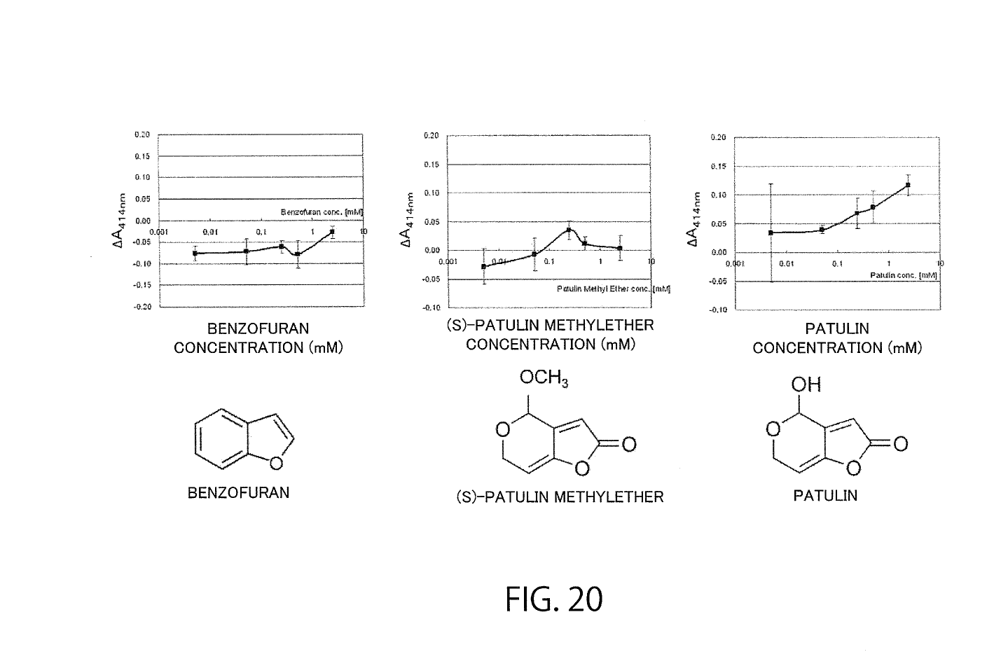

Further, in order to confirm the binding specificity of a DNA aptamer that showed high sensitivity, the binding specificity was confirmed with a patulin DNA aptamer having the base sequence of SEQ ID NO: 23 and with benzofuran and (S)-patulin methylether, which have a similar structure to that of patulin (FIG. 20). Then, it was revealed that the obtained patulin DNA aptamer does not bind to benzofuran and (S)-patulin methylether and shows binding specificity for patulin (FIG. 20).

### Example C1

### Obtainment of Patulin RNA Aptamer Molecule

In this example, obtainment of an RNA aptamer molecule binding to patulin was attempted.

As a patulin included in the detection, patulin manufactured by Wako Pure Chemical Industries, Ltd. was used. For a patulin RNA aptamer molecule, a method for screening for RNA molecules from a pool of random RNAs of 35 nucleotide having about 6×10¹⁴ varieties using the binding to patulin as an index was employed. In order to detect the binding to patulin, an RNA molecule was incorporated into a self-cleaving ribozyme to be synthesized (see FIG. 21A), and by detecting the self-cleavage occurring patulin-binding-dependently, a patulin RNA aptamer was screened. In the screening, using the modified SELEX method (Makoto Koizumi et al., Nature Structural Biology (1999) 6: 1062-1071), obtainment of an RNA aptamer having high patulin binding was attempted.

With regard to an RNA construct in which a random RNA of 35 nucleotides having about 6×10¹⁴ varieties is incorporated into a self-cleaving ribozyme region (see FIG. 21A), PCR was performed for a template DNA obtained by chemosynthesis (SEQ ID NO: 27), using a forward primer (SEQ ID NO: 28) and a reverse primer (SEQ ID NO: 29).

PCR was performed by repeating a cycle of 94° C. for 30 seconds and 61° C. for 30 seconds 4 times using Taq DNA Polymerase (Funakoshi Corporation, product number: E00007), a DNA polymerase, and with the composition in Table 8.

TABLE 8
Composition of reaction solution for PCR

| Component | Amount | |
|---|---|---|
| cDNA pool | 25 | μg |
| Forward primer | 10 | μM |
| Reverse primer | 10 | μM |
| dNTP | Each 200 | μM |
| Tag DNA Polymerase | 200 | μL |
| 10 × reaction buffer | 1 | mL |
| (including DNA polymerase) | | |
| Milli-Q water | 6.3 | mL |
| Total | 10 | mL |

A template DNA and primers used for PCR were as shown in Table 9.

TABLE 9
Table 9: General sequence of template DNA and
the sequence of primers used for PCR

| | | SEQ |
|---|---|---|
| Sequence | | ID |
| name | Base sequence | NO |
| Template | 5′-GGGCAACCTACGGCTTTCACCGTTTCG(N₃₀) | 27 |
| DNA | CTCATCAGGGTCGCC-3′ | |
| Forward | 5′- | 28 |
| primer | TAATACGACTCACTATAGGGCGACCCTGATGAG-3′ | |
| Reverse | 5′-GGGCAACCTACGGCTTTCACCGTTTCG-3′ | 29 |
| primer | | |

By transcribing 100 μg of the obtained PCR product using T7 RNA Polymerase (Takara Bio Inc.), an RNA molecule was synthesized.

The patulin-dependent self-cleavage of RNA was detected as follows. In other words, 1 μM of RNA pool was dissolved in 50 mM Tris-HCl pH 7.5, and after the RNA was heated at 95° C. for 2 minutes in order to make an aptamer fold a correct conformation, it was cooled at room temperature for 30 minutes to 2 hours.

In order to remove molecules that are self-cleaved even in the absence of patulin (negative selection), first, in the absence of patulin, 20 mM of MgCl₂ was added, and then RNA was incubated at room temperature for 30 minutes to 12 hours. With 10% modified PAGE (8M urea-containing polyacrylamide gel electrophoresis) separation, a patulin-non-binding RNA that is self-cleaved even in the absence of patulin was removed. An RNA that was not self-cleaved was clearly separated as a band from an RNA that was self-cleaved, and easily recovered by cutting a gel to be eluted. Subsequently, the recovered RNA was dissolved in 50 mM Tris-HCl pH 7.5 so that the concentration was 1 μM. Then, in order to make an aptamer fold a correct conformation, it was heated at 95° C. for 2 minutes again, and was cooled at room temperature for 30 minutes to 2 hours. Then, a solution containing patulin and 20 mM of Mg²⁺ was added, and the RNA was incubated at room temperature for minutes to 30 minutes to induce the patulin-dependent self-cleavage of an RNA molecule. After incubation, again with modified PAGE, an RNA molecule that was self-cleaved by being bound to patulin was recovered.

For RNA contained in a pool of the recovered patulin RNA aptamers, by performing reverse transcription using Superscript III (Invitrogen Corporation), a cDNA pool was obtained. PCR was performed for the obtained cDNA pool with a forward primer of SEQ ID NO: 28 containing a T7 promoter sequence and a reverse primer of SEQ ID NO: 29 containing a deficient sequence due to self-cleavage, and a DNA pool containing a T7 promoter sequence and a sequence before self-cleavage was obtained. Then, the PCR product was transcribed with T7 RNA Polymerase (Takara Bio Inc.), and an RNA pool used for the next round was obtained.

The above mentioned cycle of folding, negative selection, positive selection, modified PAGE, RT-PCR, and transcription into RNA molecule (modified SELEX method) was performed for 11 rounds, and a DNA molecule encoding a patulin RNA aptamer was obtained. The obtained DNA molecule was cloned with TOPO TA Cloning Kit For Sequensing (Invitrogen Corporation), and the base sequence was determined.

When the sequence of the patulin RNA aptamer and the RNA construct was estimated from the DNA molecule thus obtained, an RNA construct of SEQ ID NO: 30 containing a patulin RNA aptamer of SEQ ID NO: 33 and an RNA construct of SEQ ID NO: 31 containing a patulin RNA aptamer of SEQ ID NO: 34 were obtained. A cycle of the modified SELEX method was performed for 9 rounds using a template of another pool with the same methods as mentioned above, and an RNA construct of SEQ ID NO: 32 containing a patulin RNA aptamer of SEQ ID NO: 35 was obtained.

The sequence of the obtained RNA construct was as shown in Table 10.

TABLE 10
Table 10: Base sequence of RNA construct
containing patulin RNA aptamer and
self-cleaving ribozyme

| | SEQ |
|---|---|
| Base sequence of the full length | ID |
| of self-cleaving ribozyme | NO |
| 5′-GGGCGACCCU GAUGAGAAAG AUCUACAGCA | 30 |
| AAAACCAUAG UAGUAAAGAA GCGAAACGGU | |
| GAAAGCCGUA GGUUGCCC-3′ | |
| 5′-GGGCGACCCU GAUGAGAGUA UAAAAUAUCA | 31 |
| AUGAAAUAAA CAAGCCAUUA UCGAAACGGU | |
| GAAAGCCGUA GGUUGCCC-3′ | |
| 5′-GGGCGACCCU GAUGAGGGGG CACGCGUACG | 32 |
| GCUAGCCAAG UCAAACGAUU CCGAAACGGU | |
| GAAAGCCGUA GGUUGCCC-3′ | |

*The underlined portion in the base sequence represents the patulin RNA aptamer portion in an RNA construct.

The base sequence of the patulin RNA aptamer portion in the obtained RNA construct was as shown in Table 11.

TABLE 11
Table 11: Base sequence of patulin RNA
aptamer portion in self-cleaving ribozyme

| | SEQ |
|---|---|
| Base sequence of patulin RNA aptamer | ID |
| portion | NO |
| 5′-AAAGAUCUAC AGCAAAAACC AUAGUAGUAA AGAAG-3′ | 33 |
| 5′-AGUAUAAAAU AUCAAUGAAA UAAACAAGCC AUUAU-3′ | 34 |
| 5′-GGGGCACGCG UACGGCUAGC CAAGUCAAAC GAUUC-3′ | 35 |

When the secondary structure of a patulin RNA aptamer portion of SEQ ID NOS: 30 to 32 was analyzed with the Mfold program (http://frontend.bioinfo.rpi.edu/applications/mfold/cgi-bin/dna-f orm1.cgi), the secondary structure shown in FIG. 21 was predicted.

### Example C2

### Measurement of Detection Sensitivity of RNA Construct Containing Obtained Patulin RNA Aptamer

In this example, the patulin detection sensitivity was confirmed for the RNA construct obtained in Example C1.

With a DNA having a base sequence of the complementary strand of an RNA construct of SEQ ID NOS: 30 to 32 (U in the base sequence corresponds to T for DNA) as a template, PCR was performed using a forward primer of SEQ ID NO: 28 and a reverse primer of SEQ ID NO: 29 with the same methods as in Example C1. The obtained DNA fragments were purified with MinElute PCR Purification Kit (trade name, QIAGEN K.K.). With the obtained DNA as a template, using T7 RNA Polymerase (trade name, Takara Bio Inc.), RNA was synthesized by in vitro transcription and was purified by modified PAGE to obtain an RNA construct.

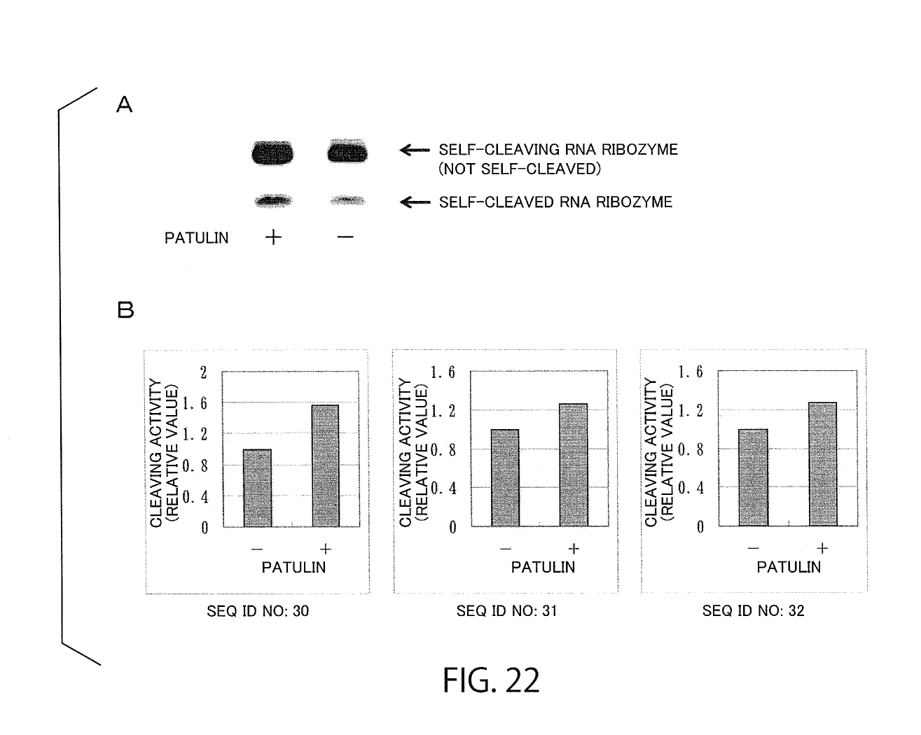

After each RNA construct (1 μM) was heated at 95° C. for 2 minutes, the RNA construct was cooled at room temperature for 30 minutes to 2 hours, and then, by incubating the RNA construct at 25° C. for 8 minutes in a buffer containing 50 mM Tris (pH 7.5) and 20 mM MgCl₂ together with 0 mM (control) or 1 mM of patulin, each RNA construct is bound to patulin, and the self-cleavage of the RNA construct was induced. The obtained reaction product was separated by modified 10% PAGE, and the RNA in a gel was visualized with Las-4000 (FUJIFILM Corporation). The intensity of each band was determined by standardizing the amount of RNA loaded on each lane using Multi Gauge Ver3.0 software (FUJIFILM Corporation). Although self-cleavage can be seen in the presence of Mg²⁺ ion even in the absence of patulin, RNA molecules that were self-cleaved were significantly increased in the presence of patulin (FIGS. 22A and B).

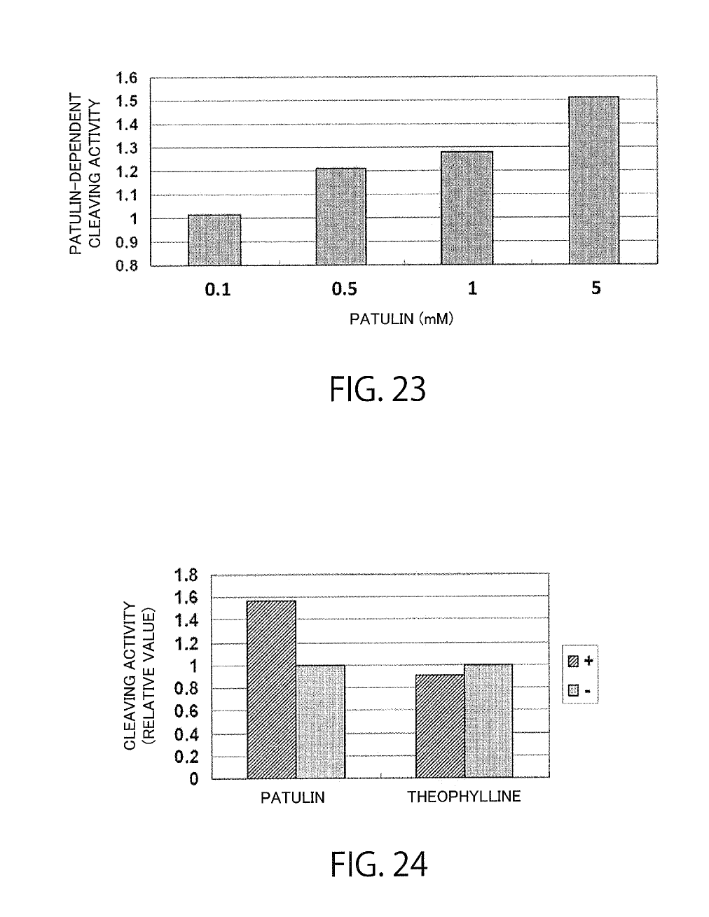

The patulin detection sensitivity was then confirmed for the obtained RNA construct having the sequence of SEQ ID NO: 30. This RNA construct can detect patulin at a concentration of 100 μM, and showed a dose-dependent increase in the self-cleaving activity at a concentration range of 100 μM to 5 mM (FIG. 23).

Furthermore, in order to confirm the binding specificity of the RNA construct for patulin, using an RNA construct having the base sequence of SEQ ID NO: 30, the binding specificity was confirmed with theophylline, which has a similar structure to that of patulin (Wako Pure Chemical Industries, Ltd.). Specifically, whether self-cleavage occurs or not was confirmed, using a reaction solution to which 1 mM theophylline was added instead of patulin. In the RNA construct, the self-cleavage occurred by patulin, while the self-cleavage did not occur by theophylline, revealing that the RNA construct shows specificity for patulin (FIG. 24).

In this example, 3 types of patulin RNA aptamer molecules having binding specificity for patulin, and an RNA construct containing a patulin RNA aptamer and a self-cleaving ribozyme could be obtained.

### Example C3

### Deletion of Bases in the Patulin RNA Aptamer Region

In this example, the self-cleaving activity when the 5′ end or the 3′ end of the RNA construct obtained in Example C1 was deleted was examined.

Four bases each at the 5′ end or the 3′ end of 2 types of patulin RNA aptamers having the base sequence of SEQ ID NO: 34 and SEQ ID NO: 35 were deleted, and the patulin RNA aptamers with the deletion were incorporated into a self-cleaving ribozyme with the same methods as in Example C1 to prepare an RNA construct.

Each of the obtained 2 types of RNA constructs was mixed with patulin in a solution containing 20 mM of Mg²⁺ (composition: 50 mM Tris (pH 7.5), 20 mM MgCl₂), and the presence or absence of self-cleavage was confirmed by gel electrophoresis.

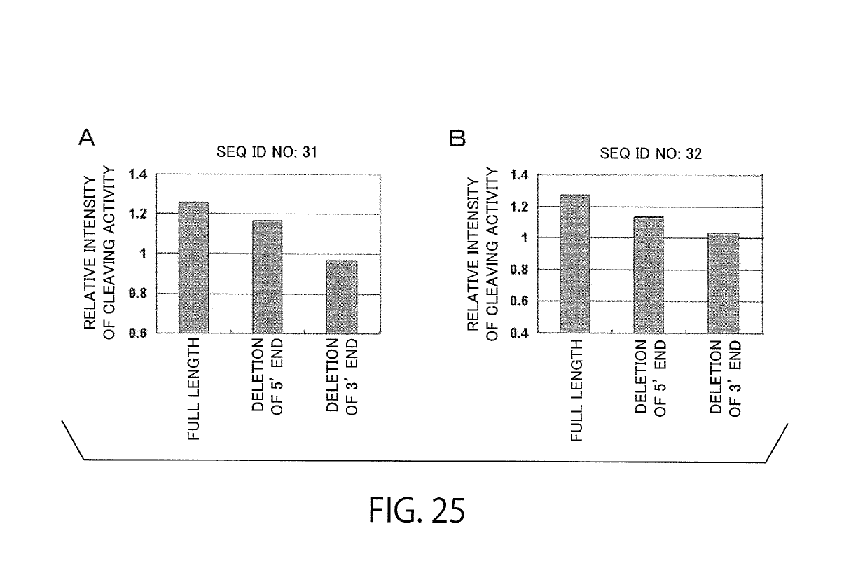

The results were as shown in FIG. 25. In both of the confirmed 2 types of RNA constructs, activity was not observed when the 3′ end of the patulin RNA aptamer portion was deleted, while self-cleaving activity was observed when the 5′ end of the patulin RNA aptamer portion was deleted. Therefore, it was demonstrated that in the confirmed 2 types of RNA constructs, the base sequence at the 5′ end of the patulin RNA aptamer portion is not essential for detection of patulin, and can be deleted. The base sequence of the patulin RNA aptamers in which 4 bases at the 5′ end of 2 types of patulin RNA aptamers having the base sequence of SEQ ID NO: 34 and SEQ ID NO: 35 is SEQ ID NO: 36 and SEQ ID NO: 37, respectively, and the base sequence of the RNA constructs obtained by incorporating these aptamers is SEQ ID NO: 38 and SEQ ID NO: 39, respectively. In other words, the sequence of the obtained RNA constructs was as shown in Table 12.

TABLE 12
Table 12: Base sequence of RNA construct
containing patulin RNA aptamer and
self-cleaving ribozyme

| | SEQ |
|---|---|
| | ID |
| Base sequence of the full length | NO |
| 5′-GGGCGACCCU GAUGAGUAAA AUAUCAAUGA AAUAAACAAG | 38 |
| CCAUUAUCGA AACGGUGAAA GCCGUAGGUU GCCC-3′ | |
| 5′-GGGCGACCCU GAUGAGCACG CGUACGGCUA GCCAAGUCAA | 39 |
| ACGAUUCCGA AACGGUGAAA GCCGUAGGUU GCCC-3′ | |

*The underlined portion in the base sequence represents the patulin RNA aptamer portion in an RNA construct.

In this way, a patulin RNA aptamer and a patulin DNA aptamer showing binding to patulin were successfully obtained. It could be confirmed that both of these RNA and DNA easily detect the binding to patulin by being incorporated into a nucleic acid construct using a self-cleaving ribozyme or a redox DNAzyme. These patulin RNA aptamer and patulin DNA aptamer also showed binding specificity for patulin. Therefore, it was suggested that a patulin RNA aptamer and a patulin DNA aptamer are useful in construction of a system specifically detecting only patulin in a sample.

### Example D1

### Realizing High Sensitivity of Candidate Group 2-7, DNA Construct of the Present Invention Containing Patulin Aptamer Region

In this example, in order to realize high sensitivity of detection of ligands, for the DNA construct of the candidate group 2-7 obtained in Example B2, optimization of the base sequence of the terminal region and the module region (i.e., 4 bases at the 3′ end of the aptamer mask region, the junction region 1, the junction region 2, and the patulin aptamer region) was attempted.

Specifically, first, 3 bases in the terminal region of the DNA construct of the candidate group 2-7 (SC-7) obtained in Example B2 were changed from 5′-CCC-3′ to 5′-CCCA-3′.

Patulin was detected based on absorbance in accordance with the statement in Example B2.

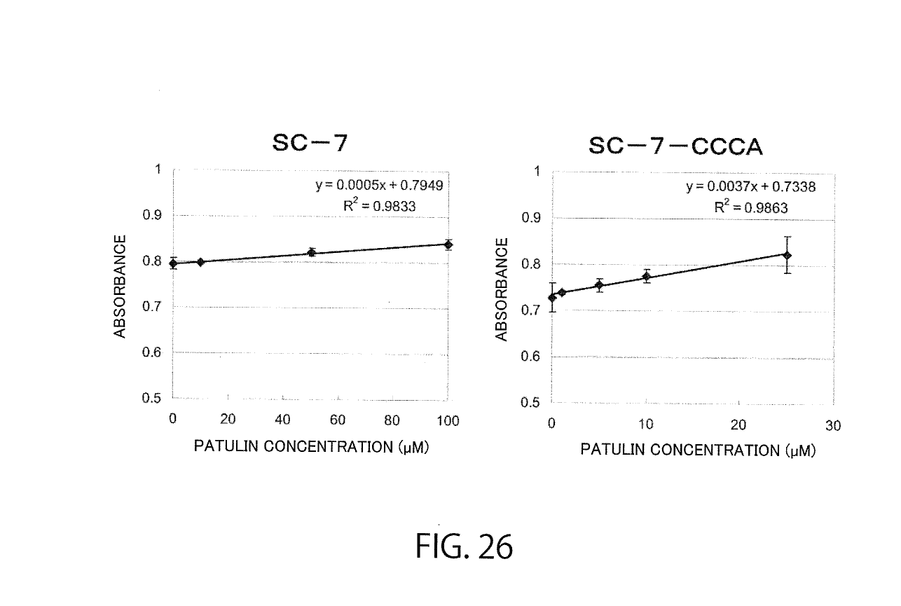

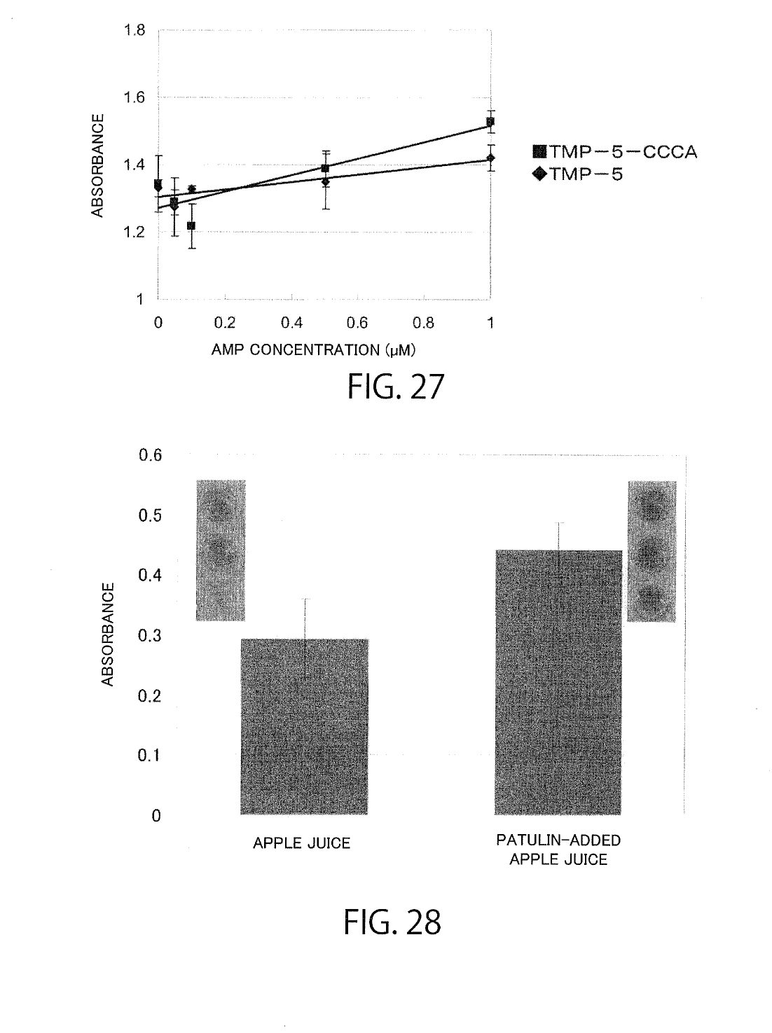

As a result, in a DNA construct in which 3 bases in the terminal region were modified to 5′-CCCA-3′, a concentration-dependent change in absorbance became remarkable (FIG. 26). The above modified DNA construct is hereinafter referred to as SC-7-CCCA (SEQ ID NO: 40). Even when TMP-5-CCCA in which 3 bases in the terminal region of TMP-5 were modified from 5′-CCC-3′ to 5′-CCCA-3′ was used, a concentration-dependent change in absorbance became remarkable (FIG. 27).

### Example D2

### Detection of Patulin in Apple Products

In this example, whether patulin in an apple juice sample can be detected using SC-7-CCCA obtained in Example D1 or not was confirmed.

The amount of patulin in apple products is used as a product quality standard. The Ministry of Health, Labour and Welfare established a patulin standard of 50 ppb or less (324 nM or less) for apple juice in November 2003 (the same as the standard by WHO) (http://www.nihs.go.jp/dmb/paturin.html). Thus, using SC-7-CCCA, detection of patulin by dissolving patulin in an actual apple juice sample so that the concentration was 300 nM (i.e., the above mentioned standard or less) was attempted.

For visual judgment by comparing with a patulin-free sample, an absorbance difference of about 0.1 is required. When patulin was added so that the final concentration was 300 nM, it was considered that an apple juice needs to be 100-fold concentrated to adjust the concentration so that it is about 30 μM in order to obtain an absorbance difference of 0.1 (e.g., see FIG. 26). Then, for patulin contained in an apple juice sample (patulin concentration of 300 nM), using a column for patulin purification marketed as POLYINTELL AFFINIMIP (trademark) patulin (apple juice), the sample was purified in accordance with the manufacture's manual.

The eluted solution was vacuum-concentrated by evaporation to dryness. The dried sample was dissolved with a reaction buffer (25 mM HEPES (pH 7.4), 20 mM KCl, 200 mM NaCl, 1% DMSO). The final concentration of patulin in the obtained solution was 20 μM (measured by high performance liquid chromatography).

Using the obtained solution, patulin was detected with the methods as mentioned in Example B2. As a result, compared with a patulin-free sample, a patulin-added sample showed an absorbance difference of about 0.1, resulting in visual detection of patulin at a concentration of 50 ppb or less of the standard in an apple juice (FIG. 28, p=0.032 for t-test).

### Example D3

### Optimization of Module Region of SC-7-CCCA

Optimization of the module region portion of SC-7-CCCA in which the sequence of the terminal region was 5′-CCCA-3′ was attempted.

The module region portion of SC-7-CCCA was designed to meet the following conditions: Condition 1: the base length of the aptamer mask region is 4 or 5,

Condition 2: the base length of the junction region 1 and 2 is 3,

Condition 3: the dG of the DNA construct calculated by secondary structure estimation in the UNAfold Version 3.8 is −12 to −6 (kcal/mol), and

Condition 4: 1 internal loop is formed between the aptamer mask region and the 3′ end of the patulin aptamer region.

As a result, 2,737 sequences in which the base length of the aptamer mask region was 4 were designed, and thus DNA was synthesized on an electrochemical detection microarray so that 1 sequence was in 5 spots, as mentioned in Example B2. However, in this example, deoxythymine (dT) of 1 base length was added to the 3′ end of DNA.

Since 8,535 sequences in which the base length of the aptamer mask region was 5 were obtained, the sequences were narrowed down to 3,753 sequences based on the below rules, and DNA was synthesized on an electrochemical detection microarray so that 1 sequence was in 3 spots, as mentioned in Example A2. In this example, deoxythymine (dT) of 1 base length was added to the 3′ end of DNA. In the narrowing down to 3,753 sequences, using the dG of the DNA construct calculated by secondary structure estimation in the UNAfold Version 3.8 as an index, DNA constructs were classified by 1 (kcal/mol) and groups were made, and from each group, (the number of sequences classified into each group) x about 3,753/8,535 sequences were randomly selected. For example, a group of DNA constructs in which dG was −8 to −7 (kcal/mol) was defined as 1 group, and a group of DNA constructs in which dG was −7 to −6 (kcal/mol) was classified as another one group.

Patulin was then detected with the methods as mentioned in Example B2. Subsequently, a DNA construct suitable for detection of patulin was selected with the following standards:

Standard 1: an electrical signal ratio of patulin concentration 10 μM to 1 μM is 2 or more, and

Standard 2: an electrical signal ratio of patulin concentration 1 μM to 0 μM is 1 or more.

Then, 11 candidate sequences were obtained with the standards 1 and 2.

Further, a DNA construct having these 11 candidate sequences was further selected using an absorbance measurement method with the following standard:

Standard 3: the standard deviation a of absorbance when a patulin concentration is 0 μM is calculated for each DNA construct, and a patulin concentration in which the absorbance increases by a or more is lower than that of SC-7-CCCA.

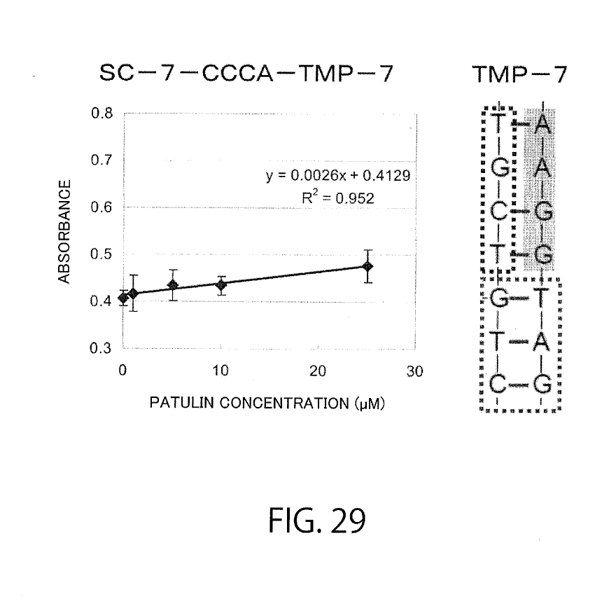

Then, 1 DNA construct (SEQ ID NO: 41) was obtained with the standard 3. The obtained DNA construct was designated SC-7-CCCA-TMP-7, and its module region was designated TMP-7. In this DNA construct, a patulin concentration in the standard 3 was 5 μM, which was lower than 10 μM for SC-7-CCCA (FIG. 29). The slope of the calibration curve for patulin was 0.0026 for this DNA construct, while that was 0.0005 for SC-7 and 0.0037 for SC-7-CCCA.

The base sequence of the obtained SC-7-CCCA and SC-7-CCCA-TMP-7 is shown in Table 13.

TABLE 13
Table 13: Base sequence of DNA construct
obtained by modifying SC-7

| | | SEQ |
|---|---|---|
| Sequence | Base sequence of patulin aptamer | ID |
| name | region portion of eachsequence | NO |
| SC-7- | 5′- | 40 |
| CCCA | GGGTAGGGCGGGTTGGGAGCTATTCCTGCGGGCG | |
| | CTGTTCGCCTAGTCGGAAGGGATCCCA-3′ | |
| SC-7- | 5′- | 41 |
| CCCA- | GGGTAGGGCGGGTTGGGCTGTCGTCCTGCGGGCG | |
| TMP-7 | CTGTTCGCCTAGTCGGAAGGTAGCCCA-3′ | |

*The underlined portion in the base sequence represents the patulin aptamer region in a DNA construct.

In this way, using a DNA construct of SEQ ID NO: 20 as a starting material, the present inventors, in Example A3, fixed the regions other than the module region and modified only the module region, and obtained a DNA construct having the base sequence of SEQ ID NOS: 1 to 15 for highly sensitive detection of AMP. Then, in Example B2, by modifying only the DNA aptamer portion of a DNA construct having the obtained TMP-5 as a module sequence, a DNA construct having the base sequence of SEQ ID NOS: 21 to 23 for detection of patulin was obtained. Furthermore, in Example D1, by modifying the terminal region of a DNA construct having the base sequence of SEQ ID NO: 23 (candidate group 2-7; SC-7), a DNA construct having the base sequence of SEQ ID NO: 40 (SC-7-CCCA) was obtained, and by modifying the module region, a DNA construct having the base sequence of SEQ ID NO: 41 (SC-7-CCCA-TMP-7) was obtained.

In this way, the present inventors could optimize the whole or part of a DNA construct, and modify at least 1 region in the DNA construct to further optimize the DNA construct. Specifically, in Examples A3 and D1, by modifying the module region, the present inventors improved the detection sensitivity for AMP and patulin of the DNA construct. In Example D1, by modifying the terminal region, the present inventors could further optimize the DNA construct.

The present inventors, in Example B2, also demonstrated that even when part of the regions of a DNA construct, i.e., the AMP aptamer region is substituted by the patulin aptamer region, optimization of the DNA construct is possible.

1.-45. (canceled)

46. A method for screening for a DNA molecule for detection of a ligand or a nucleic acid molecule having a base sequence equivalent thereto, the method comprising the following steps of:
(A) obtaining a DNA molecule candidate group for detection of a ligand or a nucleic acid molecule having a base sequence equivalent thereto by designing or modifying the base sequence of a DNA molecule, which is composed of a DNA aptamer region, an effector region that is activated dependent on ligand-binding, an aptamer mask region, a junction region 1, and a junction region 2, comprises a module region that intervenes between the DNA aptamer region and the effector region, and also forms a loop structure in the absence of the ligand,
(B) fabricating a microarray equipped with a sensor element in which a DNA molecule or a nucleic acid molecule having the designed or modified base sequence is immobilized on the electrode surface,
(C) electrochemically measuring the redox current from the effector region using the obtained microarray, and
(D) selecting a DNA molecule or a nucleic acid molecule using the detection sensitivity of a ligand as an index.

47. The method according to claim 46, which is used for optimization of the detection sensitivity of a ligand by a DNA molecule or a nucleic acid molecule.

48. The method according to claim 47, wherein at least one region selected from the DNA aptamer region, the module region, the effector region, and other region(s) is selected and the base sequence is designed or modified.

49. The method according to claim 46, wherein the base sequence of a DNA molecule candidate group is obtained using as an index the DNA construct which is any one of the followings:
(1) one forming a loop structure or a nucleic acid construct having a base sequence equivalent thereto, which comprises a DNA aptamer region, an aptamer mask region, a junction region 1, a junction region 2, an effector region, and a terminal region,
each region being connected in the order of the junction region 1, the aptamer mask region, the DNA aptamer region, and the junction region 2 from the 5′ side of the DNA construct,
at least part of the effector region being inactivated by being hybridized with the terminal region in the absence of ligands to the DNA aptamer region, and
the effector region being activated dependent on the binding of ligands to the DNA aptamer region;
wherein
4 to 7 bases at the 3′ end of the DNA aptamer region are hybridized with the aptamer mask region of 3 to 5 bases length adjacent to the 5′ side of the DNA aptamer region in the absence of ligands, to form a total of 4 to 11 hydrogen bonds between bases in the hybridized region;
the junction region 2 of 1 to 5 bases length adjacent to the 3′ side of the DNA aptamer region is hybridized with the junction region 1 adjacent to the 5′ side of the aptamer mask region in the absence of ligands, to form a total of 3 or more hydrogen bonds between bases in the hybridized region; and
the effector region is adjacent to the 5′ side of the junction region 1 and the terminal region is adjacent to the 3′ side of the junction region 2, or the effector region is adjacent to the 3′ side of the junction region 2 and the terminal region is adjacent to the 5′ side of the junction region 1, or
(2) one forming a loop structure or a nucleic acid construct having a base sequence equivalent thereto, which comprises a DNA aptamer region, an aptamer mask region, a junction region 1, a junction region 2, an effector region, and a terminal region,
each region being connected in the order of the junction region 2, the DNA aptamer region, the aptamer mask region, and the junction region 1 from the 5′ side of the DNA construct,
at least part of the effector region being inactivated by being hybridized with the terminal region in the absence of ligands to the DNA aptamer region, and
the effector region being activated dependent on the binding of ligands to the DNA aptamer region;
wherein
4 to 7 bases at the 5′ end of the DNA aptamer region are hybridized with the aptamer mask region of 3 to 5 bases length adjacent to the 3′ side of the DNA aptamer region in the absence of ligands, to form a total of 4 to 11 hydrogen bonds between bases in the hybridized region;
the junction region 2 of 1 to 5 bases length adjacent to the 5′ side of the DNA aptamer region is hybridized with the junction region 1 adjacent to the 3′ side of the aptamer mask region in the absence of ligands, to form a total of 3 or more hydrogen bonds between bases in the hybridized region; and
the effector region is adjacent to the 3′ side of the junction region 1 and the terminal region is adjacent to the 5′ side of the junction region 2, or the effector region is adjacent to the 5′ side of the junction region 2 and the terminal region is adjacent to the 3′ side of the junction region 1.

50. The method according to claim 46, wherein a ligand is patulin.

51. The method according to claim 46, which further comprises, after performing screening comprising the steps (A), (B), (C), and (D) defined in claim 46, at least one screening step comprising the following steps of:
(A′) obtaining a DNA molecule candidate group for detection of ligands or a nucleic acid molecule having a base sequence equivalent thereto by modifying the DNA molecule obtained by the screening performed before,
(B) fabricating a microarray equipped with a sensor element in which a DNA molecule or a nucleic acid molecule having the designed or modified base sequence is immobilized on the electrode surface,
(C) electrochemically measuring the redox current from the effector region using the obtained microarray, and
(D) selecting a DNA molecule or a nucleic acid molecule using the detection sensitivity of a ligand as an index.

52. The method according to claim 49, wherein the aptamer mask region forms at least one bulge loop or internal loop between bases in this region and the DNA aptamer region to which the aptamer mask region hybridizes.

53. The method according to claim 49, wherein the junction region 1 forms at least one bulge loop or internal loop between bases in this region and the junction region 2.

54. The method according to claim 53, wherein the junction region 1 and the junction region 2 are 3 bases length each.

55. The method according to claim 49, wherein the aptamer mask region is 4 or 5 bases length.

56. The method according to claim 55, wherein the aptamer mask region forms 2 base pairs and a T-G mismatched base pair, or 3 or 4 base pairs between this region and the 3′ end of the DNA aptamer region or the 5′ end of the DNA aptamer region in the absence of ligands.

57. The method according to claim 49, wherein the DNA aptamer region forms hydrogen bonds between bases in this region and the aptamer mask region in the absence of ligands in 4 bases at the 3′ end of the DNA aptamer region or the 5′ end of the DNA aptamer region.

58. The method according to claim 57, wherein
the aptamer mask region is T-(X)ₙ-T-T from the 5′ side and 4 bases at the 3′ end of the DNA aptamer region is A-A-Z-G from the 5′ side when the DNA aptamer region is adjacent to the 3′ side of the aptamer mask region, or
the aptamer mask region is T-T-(X)ₙ-T from the 5′ side and 4 bases at the 5′ end of the DNA aptamer region is G-Z-A-A from the 5′ side when the DNA aptamer region is adjacent to the 5′ side of the aptamer mask region; and
n is 1 or 2, and when n is 2, two (2) Xs may be the same base or different bases and (X)ₙ and Z form an internal loop or a bulge loop, or when n is 1, X and Z are selected from a combination of bases forming an internal loop between X and Z.

59. The method according to claim 55; wherein
the aptamer mask region is 4 bases length; and
when the aptamer mask region has a mismatched base pair in the absence of ligands, the bases forming the mismatched base pair are selected from a combination of bases so that an increase in dG (ddG) of a secondary structure in the whole molecule due to the mismatched base pair in the aptamer mask region is +0.1 kcal/mol or more; and/or
when the junction region has a mismatched base pair in the absence of ligands, the bases forming the mismatched base pair are selected from a combination of bases so that an increase in dG of a secondary structure in the whole molecule due to the mismatched base pair in the junction region is +1.0 kcal/mol or less.

60. The method according to claim 49, wherein when a ligand binds to the aptamer region, part of the bases in the aptamer mask region are hybridized with the junction region 2 to form 4 or more hydrogen bonds.

61. The method according to claim 60, wherein 4 or more hydrogen bonds formed between part of the bases in the aptamer mask region and the junction region 2 are formed by 2 base pairs, 2 base pairs and a T-G mismatched base pair, or 3 base pairs.

62. The method according to claim 49, wherein when a DNA molecule forms a secondary structure, a change in free energy (dG) in the absence of ligands is −12 to −5 (kcal/mol).

63. The method according to claim 49, wherein the DNA aptamer region is a patulin aptamer.

64. The method according to claim 63, wherein the patulin aptamer has 80% or more sequence identity to the base sequence of SEQ ID NO: 24, SEQ ID NO: 25, or SEQ ID NO: 26.

65. The method according to claim 63, wherein the patulin aptamer has the base sequence of SEQ ID NO: 24, SEQ ID NO: 25, or SEQ ID NO: 26 wherein 1 to 5 bases at the end of the base sequence may be deleted.

66. The method according to claim 49, wherein the effector region is a signal-generating region that is activated dependent on the ligands to the DNA aptamer region wherein measurement of the enzymatic activity of the signal-generating region enables the detection or determination of ligands).

67. The method according to claim 49, wherein the effector region can exert 2-fold higher activity than that in the absence of ligands by being activated dependent on the binding of ligands to the DNA aptamer region.

68. The method according to claim 66 or 67, wherein the effector region is a DNAzyme.

69. The method according to claim 68, wherein the DNAzyme is a redox DNAzyme having the base sequence of SEQ ID NO: 16.

70. The method according to claim 69, wherein the base sequence is the base sequence of SEQ ID NO: 21, SEQ ID NO: 22, SEQ ID NO: 23, SEQ ID NO: 40, or SEQ ID NO: 41.
